(19) United States
(12) Patent Application Publication
(10) Pub. No.: US 2018/0353460 A1
(43) Pub. Date: Dec. 13, 2018

(54) METHODS OF TREATING UROLOGICAL DISORDERS USING SARMs

(71) Applicant: GTx, Inc., Memphis, TN (US)

(72) Inventors: Ramesh NARAYANAN, Cordova, TN (US); Jeffrey G. Hesselberg, Germantown, TN (US); Mary Ann Johnston, Memphis, TN (US); Robert H. Getzenberg, Memphis, TN (US)

(73) Assignee: GTx, Inc., Memphis, TN (US)

(21) Appl. No.: 15/912,421

(22) Filed: Mar. 5, 2018

Related U.S. Application Data
(63) Continuation-in-part of application No. 15/910,505, filed on Mar. 2, 2018.
Application No. 15/910,505 is a continuation-in-part of application No. 15/702,757, filed on Sep. 13, 2017.
Application No. 15/702,757 is a continuation of application No. 14/885,818, filed on Oct. 16, 2015.
(60) Provisional application No. 62/064,817, filed on Oct. 16, 2014.

(51) Int. Cl. A61K 31/277; A61K 31/00; A61K 31/167

(52) U.S. Cl. CPC A61K 31/277; A61K 31/00; A61K 31/167

(57) ABSTRACT

The present invention is directed to methods of treating, preventing, suppressing and/or inhibiting urological disorders such as urinary incontinence including stress urinary incontinence, urge urinary incontinence, mixed incontinence, and pelvic floor disorders by administering a SARM compound of the invention.

### CROSS REFERENCE TO RELATED APPLICATIONS

This application is a continuation-in-part of U.S. patent application Ser. No. 15/910,505, filed on Mar. 2, 2018 which is a continuation-in-part of U.S. patent application Ser. No. 15/702,757, filed on Sep. 13, 2017, which is a continuation-in-part of U.S. patent application Ser. No. 14/885,818, filed on Oct. 16, 2015, which claims the benefit of U.S. Ser. No. 62/064,817, filed on Oct. 16, 2014, which is incorporated in its entirety herein by reference.

### FIELD OF THE INVENTION

The present invention is directed to methods of treating, preventing, suppressing and/or inhibiting urological disorders such as urinary incontinence including stress urinary incontinence (SUI), urge urinary incontinence (UUI), mixed incontinence, and pelvic floor disorders by administering a SARM compound of the invention.

### BACKGROUND OF THE INVENTION

Pelvic floor disorders affect the pelvic region of patients, and they afflict millions of men and women. In women, the pelvic region includes various anatomical structures such as the uterus, the rectum, the bladder, urethra, and the vagina. These anatomical structures are supported and held in place by a complex collection of tissues, such as muscles and ligaments. When these tissues are damaged, stretched, or otherwise weakened, the anatomical structures of the pelvic region shift. Several pelvic floor disorders include cystocele, vaginal prolapse, vaginal hernia, rectocele, enterocele, uterocele, and/or urethrocele

Pelvic floor disorders often cause urinary incontinence (UI).

Urinary incontinence is defined, as loss of bladder control. The severity ranges from occasionally leaking urine when you cough or sneeze to having an urge to urinate that is so sudden and strong you do not get to the toilet in time. The cause is physiological (drop of pelvic floor usually) with a loss of the natural anatomical valve effect of controlling one's bladder adequately resulting in weak sphincter: this is often the consequence of childbirth in women. It occurs when the interior pressure of the bladder is larger than the resistance of the urethra. It is reported that urinary incontinence generally results from the decrease in ability to regulate the urethra due to drooping of bladder, extension of the pelvic muscles, including levator ani and bulbocavernosus muscles, and weakness of the urethra sphincter.

There are several types of urinary incontinence: stress incontinence occurs when body movements put pressure on the bladder suddenly; urge incontinence occurs when people cannot hold their urine long enough to get to the toilet in time due to sensitivity of bladder muscle and when bladder leaks urine due to extreme stimulus such as a medical conditions including bladder cancer, bladder inflammation, bladder outlet obstruction, bladder stones, or bladder infection; reflex incontinence occurs due to ankylosing paraplegia; overflow incontinence occurs due to flaccid paraplegia; psychogenic incontinence occurs due to dementia; and neurogenic incontinence occurs due to damage to the nerves that govern the urinary tract.

Stress incontinence occurs when urine leaks during exercise, coughing, sneezing, laughing, lifting heavy objects, or other body movements that put pressure on the bladder. It is the most common type of bladder control problem in younger and middle-age women. In some cases, it is related to the effects of childbirth. It may also begin around the time of menopause.

Stress urinary incontinence (SUI) can coexist with urge urinary incontinence (UUI) and is then referred to as mixed urinary incontinence. UUI is part of a complex known as overactive or oversensitive bladder, which includes symptoms of frequency and/or urgency with or without UUI. 75% of patients with incontinence are elderly females.

Stress urinary incontinence (SUI), the involuntary leakage of urine during activities that increase abdominal pressure (e.g. coughing, sneezing, physical exercise), affects up to 35% of adult women (Luber K M. The definition, prevalence, and risk factors for stress urinary incontinence. Rev Urol (suppl.) 2004; 6: S3). Urinary incontinence and pelvic floor disorders are major health problems for women especially as they age.

There are a variety of treatments that may be used to treat SUI in women (Rovner E S, Wein A J. Treatment options for stress urinary incontinence. Reviews in Urology 2004, 6: S29-S47). Behavioral modification and pelvic floor physical therapy are common initial treatment approaches even though surgical procedures (e.g. sling; bladder neck suspension) are often ultimately the most effective. Biological and other materials for injection into the urethra have also been marketed for treating intrinsic sphincter deficiency (ISD), a cause of SUI symptoms. In a study of autologous fat injected into the urethral sphincter only 22% of patients improved compared to 21% after placebo injection (Lee P E, Kumg R C, Drutz H P. Periurethral autologous fat injection as a treatment for female stress urinary incontinence—a randomized double-blind controlled trial. J Urol 2001, 165: 153-158). However, the injection of muscle derived stem cells (AMDC) is a promising new therapy for SUI currently being tested in clinical trials. In a dose escalation study of AMDC, injected into the urethral sphincter, all dose groups had significantly fewer diary stress leaks at 12 months, but only patients who received the highest dose of AMDC had statistically significant reduction in mean pad weight (Peters K M, Dmochowski R R, Carr L K, Magali R, Kaufman M R, Sirls L T, Herschorn S, Birch C, Kultgen P L, Chancellor M B. Autologous muscle derived cells for treatment of stress urinary incontinence in women. J Urol 2014, 192: 469-476.). Pharmacologic therapies for SUI also have been tested with varying results. In a study of duloxetine (a selective serotonin reuptake inhibitor), the median incontinence episode frequency decreased 41% in the placebo group compared to 54% receiving duloxetine 20 mg/day, 59% for duloxetine 40 mg/day, and 64% for duloxetine 80 mg/day (Norton P A, Zinner N R, Yalcin I, Bump R C. Duloxetine urinary incontinence study group. Duloxetine versus placebo in the treatment of stress urinary incontinence. Am J Obstet Gynecol 2002, 187: 40-48). Dmochowski and colleagues also demonstrated a statistically significant reduction in incontinence episode frequency with duloxetine therapy compared with placebo (50% vs 27%, respectively) (Dmochowski R R, Miklos J R, Norton P A, et al. for the duloxetine urinary incontinence study group. Duloxetine versus placebo for the treatment of North America women with stress urinary incontinence. J Urol 2003, 170: 1259-1263).

Pelvic floor muscle relaxation has been found to correlate with lower urinary tract symptoms (LUTS). Muscles of the pelvic floor and lower urinary tract are crucial for supporting the pelvic organs and micturition, however damage to the muscles or lack of hormonal stimulation are thought to contribute to prolapse and urinary incontinence. As such, efforts have been made to improve pelvic floor muscle strength and function especially in post-reproductive and elderly women, to improve, if not cure, LUTS (specifically urinary incontinence, urinary frequency and nocturia). However, pelvic floor physical therapy (PT) is often less effective than more aggressive treatment such as surgery (Labrie J, Berghmans BLCM, Fischer K, Milani A, van der Wijk I, et al. Surgery versus physiotherapy for stress urinary incontinence. NEJM 2013, 369, 1124-1133). A prospective randomized trial of PT vs. surgery showed that 49% of women in the PT group crossed over to surgical treatment. Others have shown that after 3 to 15 years, 25 to 50% of women initially treated with physiotherapy have proceeded to surgery (Cammu H, Van Lylen M, Blockeel C, Kaufman L, Amy J-J. Who will benefit from pelvic floor muscle training for stress urinary incontinence? Am J Obstet Gynecol 2004, 191: 1152-1157; Lamers B H C, van der Vaart C H. Medium-term efficacy of pelvic floor muscle training for female urinary incontinence in daily practice. Int Urogynecol J Pelvic Floor Dysfunct 2007, 18: 301-307; Kvarstein B K, Nygaard I. Lower urinary tract symptoms and pelvic floor muscle exercise adherence after 15 years. Obstet Gynecol 2005, 105: 999-1005). Yet, surgery is much more invasive and is associated with risk and complications (Brubaker L, Norton P A, Albo M E, Chai T C, Dandreo K J. Adverse events over two years after retropubic or transobturator midurethral sling surgery: findings from the Trial of Midurethral Slings (TOMUS) study. Am J Obstet Gynecol 2011, 205: 498.e1-498.e6).).

Androgen supplementation may be a novel treatment to augment pelvic floor muscle response and improve objective and subjective outcomes for SUI. Basic science literature indicates that smooth muscle cells in various female urogenital tissues have expressed androgen receptors (Berman J R, Almeida F G, Jolin J, et al. Correlation of androgen receptors, aromatase, and 5-alpha reductase in the human vagina with menopausal status Fertil Steril 2003, 79: 925-931) and that the levator ani and urethral sphincter, both containing large numbers of androgen receptors (Copas P, Bukovsky A, Asbury B, et al. Estrogen, progesterone, and androgen receptor expression in levator ani muscle and fascia. J Women Helath Gend Based Med 2001, 10: 785-795; Celayir, S, Ilce Z, Dervisoglu S. The sex hormone receptors in the bladder in childhood-1: Preliminary report in male subjects. Eur J Pediatr Surg 2002, 12: 312-317), are sensitive to androgens (Nnodim J O. Quantitative study of the effects of denervation and castration on the levator ani muscle of the rat Anat Rec 1999, 255: 324-333; Nnodim J O. Testosterone mediates satellite cell activation in denervated rat levator ani muscle. Anat Rec 2001, 263: 19-24).

### Androgen Receptors in the Pelvic Floor/Urethra

The para-urethral extracellular matrix is a target for sex steroid hormones, however the effects are not well known. Androgens stimulate collagen synthesis and inhibit degradation leading to increased collagen fiber compactness (Shin M H, Rhie G E, Park C H, Kim K H, Cho K H, Eun H C et al. Modulation of collagen metabolism by the topical application of dehydroepiandrosterone to human skin. J Invest Dermatol 2005, 124: 315-323; Berger L, El-Alfy M, Martel C, Labrie F. Effects of dehydroepiandrosterone, Premarin and Acolbifene on histomorphology and sex steroid receptors in the rat vagina. J Steroid Biochem Mol Biol 2005, 96: 201-215). Androgen receptors are densely expressed in both muscle and stromal cells of the levator ani muscle and fascia in women (Copas P, Bukovsky A, Asbury B, et al. Estrogen, progesterone and androgen receptor expression in levator ani muscle and fascia. J Women Health Gend Based Med 2001, 10: 785-795) and the levator ani muscle is considered to be one of the most androgen sensitive tissues in the body.

### Impact of Anabolic Steroids

The effect of testosterone on urodynamic findings and histopathomorphology of the pelvic floor muscles has been studied in rat models of SUI. Testosterone was found to improve leak point pressures and significantly increase the size of myofibers in treated rats, suggesting that testosterone has both preventative and curative effects on rat models of SUI (Mammadov R, Sinsir A, Tuglu I, Eyren V, Gurer E, Ozyurt C. The effect of testosterone treatment on urodynamic findings and histopathomorphology of pelvic floor muscles in female rats with experimentally induced stress urinary incontinence. Int Urol Nephrol 2011, 43: 1003-1008). Since free testosterone levels were also higher in the treated group, there is potential for concerns regarding side effects of supplemental steroidal testosterone in women with SUI.

The anabolic effects of androgens in men have been widely studied, but less is known about the role and use of androgens in women. Prior studies have found that urinary levels of androgens were significantly higher in postmenopausal patients with SUI than in postmenopausal patients without incontinence (Jung B H, Bai S W, Chung B C. Urinary profile of endogenous steroids in postmenopausal women with stress urinary incontinence. J Reprod Med 2001, 46: 969-974). Furthermore, concentrations of androgen metabolites in urine of these patients were related positively to the bladder neck descensus when measured by perineal ultrasound (Bai S W, Jung Bh, Chung B S, et al. Relationship between urinary profile of the endogenous steroids and postmenopausal women with stress urinary incontinence. Neurourol Urodynam 2003, 22: 198-204). Aizawa K et al. and others have published data demonstrating that increases in muscle mass due to resistance training or exercise is due, at least in part, to increases in local androgen concentrations and expression of androgen-synthesizing enzymes (Aizawa K, Iemitsu M, Maeda S, Mesaki N, Ushida T, Akimoto T. Endurance exercise training enhances local sex steroidogenesis in skeletal muscle. Medicine and science in sports and exercise 2011, 43(11): 2072-2080). These findings support the notion that pelvic floor muscle strengthening exercise improves SUI symptoms by increasing androgen levels locally. These and other studies suggest that androgens may play a substantial role in SUI and that androgen metabolites might be involved in the relaxation of bladder muscle (Bai S W, Jung Bh, Chung B C, et al. Relationship between urinary endogenous steroid metabolites and lower urinary tract function in postmenopausal women. Yonsei Med J 2003, 44: 279-287). This relaxation effect on the bladder may be related to the up regulation of nitric oxide synthase by androgens to produce more nitric oxide. The action of androgen on the lower urinary tract and pelvic floor is complex and may depend on anabolic effects, hormonal modulation, receptor expression, nitric oxide modulation, or a combination of these factors (Ho M H, Bhatia N N, Bhasin S. Anabolic effects of androgens on muscles of female pelvic floor and lower urinary tract. Current Opinion in Ostetrics & Gynecology 2004, 16(5): 405-409).

Intriguing data come from studies conducted in women with polycystic ovarian syndrome (PCOS). PCOS is a hyper-androgenic disorder (>70 ng/dL compared to 15-50 ng/dL in normal pre-menopausal women) and clinical studies have demonstrated that PCOS can eliminate the increased risk for UI observed in obese women. Furthermore, obese women with PCOS have a similar prevalence of UI as those considered to have a normal body mass index (Montezuma T, Antonio F I, Rosa de Silva A C, Sa M F, Ferriani R A, Ferreira C H. Assessment of symptoms of urinary incontinence in women with polycystic ovary syndrome. Clinics (Sao Paulo, Brazil) 2011, 66(11): 1911-1915). In a separate study, none of the women with PCOS (18.6% with UI) suffered from UI compared to matched controls, though pelvic floor muscle strength was not different (Antonio F I, Bo K, Ferriani R A, Sa M F, Rosa de Silva, A C, Ferreira C H. Pelvic floor muscle strength and urinary incontinence in hyperandrogenic women with polycystic ovary syndrome. Int Urogynecol J 2013, 24(10): 1709-1714). These studies support the hypothesis that women with higher androgen levels, or potentially women treated with a selective androgen receptor modulator (SARM) will show improvements in UI symptoms.

### Selective Androgen Receptor Modulators

Although anabolic steroids may increase muscle mass and strength, lack of oral bioavailability and known potential risks have limited their use. Selective androgen receptor modulators (SARMs) have great potential to achieve similar benefits of anabolic steroid therapy (improved muscle mass, cholesterol/triglyceride levels, glucose metabolism, and bone density) with fewer adverse effects, such as hirsutism and acne, in women.

SARMs may provide a new therapeutic option for pelvic floor and lower urinary tract disorders, as both testosterone and its more potent metabolite converted by 5-α reductase, dihydrotestosterone (DHT), have anabolic effects on muscle. The potential for SARMs as a treatment for SUI is strengthened by studies showing that urethral closure pressure is the factor most strongly associated with SUI (Delancey J O, Miller J M, Kearney R, Howard D, Reddy P, Umek W, Guire K E, Margulies R U, Ashton-Miller J A. Vaginal birth and de novo stress incontinence: Relative contributions of urethral dysfunction and mobility. Obstet Gynecol 2007 (2Pt I): 354-362; Delancy J O, Trowbridge E R, Miller J M, Morgan D M, Guire K, Fenner D E. Weadock W J, Ashton-Miller J A. Stress urinary incontinence: Relative importance of urethral support and urethral closure pressure. J Urol 2008, 179(6): 2286-2290). This finding is supported by both morphological and electromyographic (EMG) evidence. In imaging studies, the striated urethral sphincter has been found to be smaller in women with SUI compared to continent controls (Athanasiou S, Khullar V, Boos K, Salvatore S, Cardozo L. Imaging the urethral sphincter with three-dimensional ultrasound. Obstet Gynecol 1999, 94(2): 295-301; Morgan D M, Umek W, Guire K, Morgan H K, Garabrant A, DeLancey J O. Urethral sphincter morphology and function with and without stress incontinence. J Urol 2009, 182(1): 203-209). In EMG studies, the striated urethral sphincters of women with SUI exhibit smaller EMG amplitudes and shorter motor-unit-potential durations, with more phases, than continent controls (Kenton K, Mueller E, Bmaker L. Continent women have better urethral neuromuscular function than those with stress incontinence. Int Urogynecol J. 2011, 22(12): 1479-1484; Takahashi S, Homnia Y, Fujishiro T, Hosaka Y, Kitamura, T, Kawabe K. Electromyographic study of the striated urethral sphincter in type 3 stress incontinence: Evidence of myogenic-dominant damages. Urology 2000, 56(6): 946-950), indicating primarily myogenic changes. Furthermore, it is well accepted that pelvic floor muscle (PFM) rehabilitation is an effective treatment for SUI (Hay-Smith J, Berghmans B, Burgio K, Dumoulin C, Hagen S, Moore K, Nygaard I, N'dow J (2009) Committee 12 adult conservative management. In: Abrams P, Cardozo L, Khoury S, Wein A (eds) Incontinence, 4^{th} Ed. Health Publications Ltd., Paris). PFM rehabilitation may be effective because it strengthens not only the pelvic floor but may also strengthen the striated urethral sphincter. This idea is supported by a recent publication that reported, based on ultrasound (US), a 12-week PFM exercise program produced a significant increase in the cross-sectional area of the urethra, at the level of the striated urethral sphincter, in middle-aged women (McLean L, Varette K, Gentilcore-Saulnier B, Harvey M A, Baker K, Sauerbrei E. Pelvic floor muscle training in women with stress urinary incontinence causes hypertrophy of the urethral sphincters and reduces bladder neck mobility during coughing. Neurourol Urodyn 2013, 32(8): 1096-n02. doi: 10.1002/nau.22343). Because of limits in the resolution of US images, the authors were not able to determine which part of the urethra hypertrophied. The investigators suggested that PFM rehabilitation in older women with SUI could condition the striated urethral sphincter leading to measurable hypertrophy, observable on MRI.

In a later study, Madill extended the findings of the McLean group (Madill S J, Pontbriand-Drolet S, Tang A, Dumoulin C. Changes in urethral sphincter size following rehabilitation in older women with stress urinary incontinence. Int Urogynecol J 2014, September, epub ahead of print). Using MRI, they were able to differentiate between smooth and striated sphincter muscle layers and determined that changes occurred primarily in the striated urethral sphincter of older women. These findings suggest that not only does the striated urethral sphincter contract synergistically with PFMs during voluntary and automatic contractions [Nnodim J O. Quantitative study of the effects of denervation and castration on the levator ani muscle of the rat. Anat Rec 1999, 255: 324-333; Nnodim J O. Testosterone mediates satellite cell activation in denervated rat levator ani muscle. AnatRec 2001, 263: 19-24; Celayir, S, Ilce Z, Dervisoglu S. The sex hormone receptors in the bladder in childhood-1: Preliminary report in male subjects. Eur J Pediatr Surg 2002, 12: 312-317], but also that PFM rehabilitation stresses the striated urethral sphincter sufficiently to produce a muscular hypertrophy training effect (Madill S J, Pontbriand-Drolet S, Tang A, Dumoulin C. Changes in urethral sphincter size following rehabilitation in older women with stress urinary incontinence. Int Urogynecol J 2014, September, epub ahead of print).

Selective androgen receptor modulators (SARMs) are currently in development for patients with muscle wasting secondary to cancer diagnosis. This class of drugs has been shown to stimulate the growth of skeletal muscle, similar to traditional anabolic steroids, but without undesirable side effects. SARMs, such as compound of Formula IX, are orally bioavailable and tissue-selective, whereas testosterone and other anabolic steroids also have limited oral bioavailability and are only available in transdermal and intramuscular formulations potentially leading to skin reactions and fluctuations in serum concentrations of testosterone. SARMs may exhibit the beneficial effects of anabolic agents without the known associated risks (Mohler M L, Bohl C E, Jones A, et al. Nonsteroidal selective androgen receptor modulators (SARMs): Dissociating the anabolic and androgenic activities of the androgen receptor for therapeutic benefit. J Med Chem 2009, 52(12): 3597-3617).

Female and male urogenital tissues robustly express androgen receptor (AR). Androgens have anabolic actions on these tissues, including the levator ani and bulbocavernosus muscles, which are pelvic floor muscles. Anabolic effects of androgens may play an important role in preventing and treating urological disorders including urinary incontinence, lower urinary tract disorders and pelvic floor disorders. Most current treatments for urinary incontinence (UI) modulate the nervous system, and include non-selective anti-cholinergics such as oxybutynin and propantheline, or anti-muscarinics such as tolterodine, trospium, solifenacin, darifenacin, and fesoterodine. Adrenergic modulators for UI include tricyclic anti-depressants (e.g., imipramine and amitriptyline) and β₃-adrenergic agonists (e.g., mirabegron). Other UI agents are muscle relaxants (e.g., relax the detrusor) such as flavoxate and dicyclomine. Botulinum toxins such as onabotulinumtoxin A have been used in neurogenic UI. Despite the number of FDA approved agents for treating UI, there remains a need for new agents with novel mechanisms of action. The use of nonsteroidal androgens to strengthen the pelvic floor and support urogenital structures is one such novel approach to treating UI.

Recently utilizing an ovariectomized rat model to mimic SUI by disrupting urethral continence, investigators demonstrated that the use of a SARM (GSK2849466A) was able to increase urethral baseline pressure (UBP) and the amplitude of urethral responses during sneezing (AURS) by 64% and 74%, respectively, as compared with the vehicle control. Further, all of the rats (8/8) in the vehicle treated group experience fluid leakage during sneezing whereas only one of the rats (1/8) in the SARM treated group experienced such leakage upon similar challenge. Histologically, the SARM treated animals had a reversal of the atrophy in urethral muscle observed in the control group. This preliminary in vivo study provides further support to the potential use of SARMs for the treatment of SUI (Kadekawa et al., AUA Annual Meeting 2015, New Orleans, La. PD27-11).

### SUMMARY OF THE INVENTION

In one embodiment, this invention provides a method of treating, preventing, suppressing or inhibiting stress urinary incontinence, urge urinary incontinence or a combination thereof in a postmenopausal female subject, comprising administering to said subject a composition comprising a SARM compound of Formula IX:

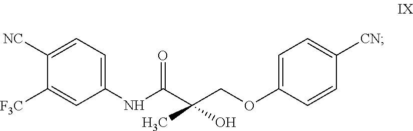 [SMILES: C[C@](O)(COc1ccc(C#N)cc1)C(=O)Nc1ccc(C#N)c(C(F)(F)F)c1]

or its isomer, hydrate, pharmaceutically acceptable salt, pharmaceutical composition or any combination.

In one embodiment, this invention provides a method of treating, preventing, suppressing or inhibiting stress urinary incontinence, urge urinary incontinence or a combination thereof in a postmenopausal female subject, comprising administering to said subject a composition comprising a SARM compound of Formula IX:

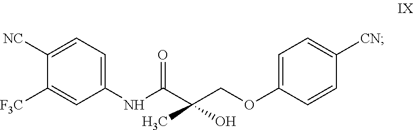 [SMILES: C[C@](O)(COc1ccc(C#N)cc1)C(=O)Nc1ccc(C#N)c(C(F)(F)F)c1]

or its isomer, hydrate, pharmaceutically acceptable salt, pharmaceutical composition or any combination thereof at a daily dose of between 1 to 3 mg.

In one embodiment this invention provides a method of reducing the occurrence or lessening the severity of at least one of the following symptoms in a postmenopausal female subject suffering from stress urinary incontinence, urge urinary incontinence or a combination thereof: (i) average daily frequency of urination; (ii) average nightly frequency of urination; (iii) total urinary incontinence episodes; (iv) stress incontinence episodes; and (v) urinary urgency episodes; comprising administering a SARM compound of Formula IX:

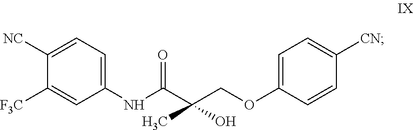 [SMILES: C[C@](O)(COc1ccc(C#N)cc1)C(=O)Nc1ccc(C#N)c(C(F)(F)F)c1]

or its isomer, hydrate, pharmaceutically acceptable salt, pharmaceutical composition or any combination.

In one embodiment this invention provides a method of reducing the occurrence or lessening the severity of at least one of the following symptoms in a postmenopausal female subject suffering from stress urinary incontinence, urge urinary incontinence or a combination thereof: (i) average daily frequency of urination; (ii) average nightly frequency of urination; (iii) total urinary incontinence episodes; (iv) stress incontinence episodes; and (v) urinary urgency episodes; comprising administering a SARM compound of Formula IX:

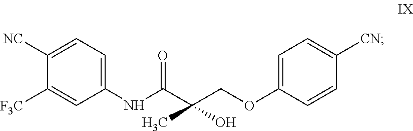 [SMILES: C[C@](O)(COc1ccc(C#N)cc1)C(=O)Nc1ccc(C#N)c(C(F)(F)F)c1]

or its isomer, hydrate, pharmaceutically acceptable salt, pharmaceutical composition or any combination thereof at a daily dose of between 1 to 3 mg.

In some embodiments, the subject suffers from stress urinary incontinence and urge urinary incontinence.

In some embodiments, the methods of this invention comprise administering a composition comprising Compound IX at a daily dose of between 1 to 3 mg. In other embodiments, the daily dose is 1 mg, 2 mg or 3 mg. Each represents a separate embodiment of this invention. In other embodiments, the administering of the composition of compound IX is for up to 12 weeks. In other embodiments, the administering step is by intermittent dosing cycles. In other embodiments, the administering step consists of only one dose cycle lasting up to 12 weeks. In other embodiments, the duration of response following completion of the treatment is at least seven months post-treatment.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts increased levator ani muscle weight in rats treated with SARM compounds (compound of Formula X, compound of Formula IX (Compound IX)) of this invention compared to an inactive compound which is structurally analogous to a SARM (R-isomer of compound of Formula IX or (R)-IX): Sprague Dawley rats (n=5; 200 g weight) were castrated and treated subcutaneously for 14 days with vehicle (open bars), 3 mg/day compound of Formula X (dotted bars), compound of Formula IX (hatched bars), an inactive (R)-IX (grey bars) and DHT (black bars). At sacrifice, organs were weighed and expressed as raw organ weights. Values are expressed as average±S.D. Increased size and strength of pelvic floor muscles is one mechanism by which SARMs are believed to affect UI.

FIG. 2 depicts tissue selective pharmacologic effects of Compound XI as described in Example 10.

FIG. 3 depicts results of Hershberger assays of compounds of the invention as described in Example 17. AUC is area under the concentration-time curve.

FIG. 4 depicts the effect of SARMs on body weight. Body weight was measured on days 0 (baseline) and 28 (post-treatment) of treatment in mice that were ovariectomized and treated with two SARMs (S-isomer of Formula IX (Compound IX (IX)) and Formula VIII (VIII)). No statistical difference was observed in body weight between the groups (n=5-7/group); mpk is mg of drug per kg body weight; B.wt. is body weight.

FIG. 5A and FIG. 5B depicts the effect of SARMs on lean body mass as measured by magnetic resonance imaging (MRI). Lean mass was measured on days 0 (baseline) and 28 (post-trt) of treatment in mice that were ovariectomized and treated with SARMs (S-isomer of Compound IX (IX) and Formula VIII (VIII)). Though a trend of increased lean mass with dose was observed in SARM-treated groups, treatment groups did not attain statistical significance (n=5-7/group). VIII was more potent than Compound IX in increasing lean mass (comparison of average between groups and raw lean mass). ‘Lean’ indicates lean body mass; mpk is mg of drug per kg body weight

FIG. 6 depicts the effect of SARMs on coccygeus (COC; a.k.a. ischiococcygeus) muscle weight. After twenty-eight days of treatment, animals were sacrificed, pelvic floor muscles isolated under magnification and weighed in a microbalance. COC was highly regulated by ovariectomy (OVX) with ˜50% reduction in weight. N=10-14 (COC muscles from both sides of pelvic floor were isolated and weighed). All groups were statistically different from OVX animals. However, no difference was observed between treatment groups. As is clearly evident from the P values, compound of Formula VIII is more potent than Compound IX. Veterinarian's observation under the microscope was that the COC muscles from animals treated with SARMs were more vascular than the OVX vehicle-treated controls or even the intact control animals. mpk is mg of drug per kg body weight; COC is Coccygeus.

FIG. 7 depicts the effect of SARMs on pubococcygeus (Pc) muscle weight. After twenty-eight days of treatment, animals were sacrificed, pelvic floor muscle isolated under magnification and weighed in a microbalance. Pc was only modestly regulated by ovariectomy with ˜15-20% weight reduction. N=10-14 (Pc muscles from both sides of pelvic floor were isolated and weighed). All groups were statistically different from OVX animals. However, no difference was observed between groups. Due to small size of the muscle and due to minimal regulation by OVX, the data has more deviation than COC. Veterinarian's observation under the microscope was that, unexpectedly, the Pc muscles from animals treated with SARMs were more vascular than the ovariectomy control or even the intact control animals. mpk is mg of drug per kg body weight; veh is vehicle; COC is coccygeus; Pc is pubococcygeus.

FIG. 8 depicts the effect of SARMs on combined pelvic floor muscle weight. After twenty-eight days of treatment, animals were sacrificed, pelvic floor muscle isolated under magnification and weighed in a microbalance. The combined weight of the COC, Pc, and (iliococcygeus (IL) are represented here. Combined weight of levator ani and coccygeus muscle reflects the trend observed with the largest muscle (COC) with ˜50% weight reduction observed due to OVX. N=10-14 (both sides of pelvic floor muscles were isolated and weighed). All groups were statistically different from OVX animals. However, no difference was observed between groups. As is clearly evident from the P values, compound of Formula VIII (VIII) is more potent than Compound IX (IX). Veterinarian's observation under the microscope was that the PC muscles exposed to SARMs were more vascular than the ovariectomy control or even the intact control animals.

FIG. 9 depicts the expression of myostatin and FBxo32 in an RNA that was isolated from the COC muscle after 28 days of treatment. Expression of myostatin and FBxo32 was measured using real-time PCR and normalized to GAPDH.

FIG. 10 depicts clinical data for mean stress leaks/day. Data were collected in post-menopausal women with stress urinary incontinence (SUI) at baseline (day 0) and at 12 weeks of treatment with the compound of Formula IX (Compound IX). (Example 3)

FIG. 11 depicts the pelvic MRI of one post-menopausal woman with SUI at baseline and at 12 weeks (Example 3) of Compound IX treatment. The image is annotated with the width (measured in millimeters (mm)) of the levator ani of this subject at baseline and 12 weeks, which was increased by ˜20% after treatment with Compound IX for 12 weeks. This suggests that the data collected in male rats and female mice also translates to post-menopausal women with SUI. (Example 3)

FIGS. 12A, 12B, 12C and 12D depict improvements in quality of life (QOL) across all measures by Compound IX. FIG. 12A: Patient Global Impression of Severity (PGI-S); FIG. 12B: Frequency Patient Global Impression of Improvement (PGI-I);

FIG. 12C: Urogenital Distress Inventory (UDI-6); and FIG. 12D: Incontinence Impact Questionnaire (IIQ-7).

FIGS. 13A and 13B depict Medical, Epidemiologic and Social aspects of Aging (MESA) score for Compound IX. FIG. 13A: depicts the median scores for the SUI questions within MESA (MESA stress), and FIG. 13B: depicts the median scores for the UUI questions within MESA (MESA urge).

FIG. 14 depicts the effect on urge leaks/day exerted by Compound IX. The chart depicts the number of mean urge leaks per a day over the course of the study for the eleven patients that reported >0 urge leaks at baseline and had a 12 weeks assessment. The effect on urge incontinence also appears to be durable for at least 7 months (N=7).

FIG. 15 depicts female sexual function index (FSFI) at 16 weeks, i.e., 4 weeks after the 12 weeks of treatment with Compound IX.

FIG. 16 depicts the pelvic MRI of three post-menopausal women with SUI at baseline and at 12 weeks (Examples 3 and 19) of Compound IX treatment. For two patients, the images are annotated with the diameter (measured in millimeters (mm)) of the urethral sphincter of these subjects at baseline and 12 weeks, which were increased after treatment with Compound IX for 12 weeks. This provides a previously unmeasured metric of Compound IX, which in post-menopausal women with SUI, demonstrated an unexpected increase in urethral size which helps to rationalize the unexpected 100% efficacy in SUI (18 of 18 patients) and UUI (11 of 11 patients) (Examples 3 and 19).

FIG. 17 depicts a 78% decrease of mean pad weight and a 71% decrease of median pad weight after 12 weeks of treatment with Compound IX.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides methods for treating, preventing, suppressing or inhibiting urological disorders. In another embodiment, this invention provides methods for: (a) treating, preventing, suppressing or inhibiting urinary incontinence (UI); (b) treating, preventing, suppressing or inhibiting pelvic floor disorders; and (c) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (d) providing androgen replacement in post-hysterectomy and post-oophorectomy women; (e) treating, preventing, suppressing or inhibiting urinary incontinence in post-hysterectomy and post-oophorectomy women; (f) treating, preventing, suppressing or inhibiting fecal incontinence; (g) increasing the size and/or weight of muscles in the pelvic floor; (h) increasing the size/strength of the urethral sphincter; (i) improving the urethral pressure profile of a subject suffering from SUI; (j) treating, preventing, suppressing or inhibiting stress urinary incontinence, urge urinary incontinence or a combination thereof in a postmenopausal female subject; and (j) improving the urethral closure pressure of a subject suffering from SUI comprising administering a SARM compound of this invention. In another embodiment, the subject is a postmenopausal female subject.

In one embodiment, non-limiting examples of urological disorders include: urinary incontinence, stress urinary incontinence, psychogenic urinary incontinence, urge urinary incontinence, reflex urinary incontinence, overflow urinary incontinence, neurogenic urinary incontinence, stress urinary incontinence caused by dysfunction of the bladder, overactive/oversensitive bladder, enuresis, nocturia, cystitis, urinary calculi, prostate disorder, kidney disorder, or a urinary tract infection.

Urological disorders include bladder overactivity that may result from detrusor instability or hyperreflexia. Triggers may include increased activity of afferent peripheral nerve terminals in the bladder or decreased inhibitory control in the central nervous system and/or in peripheral ganglia. Changes in detrusor muscle structure or function, such as increased muscle cell excitability due to denervation, may also play a role in the pathogenesis of this filling disorder.

In one embodiment, urological disorders refer to diseases of the bladder including but not limited to overactive/oversensitive bladder, overflow urinary incontinence, stress urinary incontinence caused by dysfunction of the bladder, urethra or central/peripheral nervous system.

In one embodiment, urological disorders refer to disorders of the prostate including but not limited to “a prostate disorder” which refers to an abnormal condition occurring in the male pelvic region characterized by, e.g., male sexual dysfunction and/or urinary symptoms. This disorder may be manifested in the form of genitourinary inflammation (e.g., inflammation of smooth muscle cells) as in several common diseases of the prostate including prostatitis, benign prostatic hyperplasia and cancer, e.g., adenocarcinoma or carcinoma, of the prostate.

In one embodiment, urological disorders refer to kidney disorders, cystic diseases of the kidney, cystic diseases of renal medulla, systemic disorders and diseases affecting tubules and interstitium, and other vascular disorders.

In one embodiment, this invention provides a method of treating, preventing, suppressing or inhibiting a urinary incontinence in a subject, comprising administering to said subject a SARM compound of this invention or its optical isomer, pharmaceutically acceptable salt, hydrate, or any combination thereof.

In another embodiment, urinary incontinence includes stress incontinence, urge incontinence, reflex incontinence, overflow incontinence, neurogenic urinary incontinence, psychogenic incontinence or a combination thereof. In another embodiment, urinary incontinence is stress incontinence. In another embodiment, urinary incontinence is urge incontinence. In another embodiment, urinary incontinence is reflex incontinence. In another embodiment, urinary incontinence is overflow incontinence. In another embodiment, urinary incontinence is psychogenic incontinence.

Fecal incontinence is the accidental passing of solid or liquid stool or mucus from the rectum. Injury to one or both of the sphincter muscles can cause fecal incontinence. If these muscles, called the external and internal anal sphincter muscles, are damaged or weakened, they may not be strong enough to keep the anus closed and prevent stool from leaking. Trauma, childbirth injuries, cancer surgery, and hemorrhoid surgery are possible causes of injury to the sphincters.

In one embodiment, the methods of this invention include treating, preventing, suppressing or inhibiting fecal incontinence comprising administering a compound of Formulas I-XIV of this invention.

In one embodiment, this invention provides a method of reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; comprising administering a SARM compound of this invention or its optical isomer, pharmaceutically acceptable salt, hydrate, or any combination thereof.

In one embodiment, this invention provides a method of treating, preventing, suppressing or inhibiting pelvic floor disorders in a subject; comprising administering a SARM compound of this invention or its optical isomer, pharmaceutically acceptable salt, hydrate, or any combination thereof.

In another embodiment, pelvic floor disorders include cystocele, vaginal prolapse, vaginal hernia, rectocele, enterocele, uterocele, urethrocele or a combination thereof. In another embodiment, pelvic floor disorder is cystocele. In another embodiment, pelvic floor disorder is vaginal prolapse. In another embodiment, pelvic floor disorder is vaginal hernia. In another embodiment, pelvic floor disorder is rectocele. In another embodiment, pelvic floor disorder is enterocele. In another embodiment, pelvic floor disorder is uterocele. In another embodiment, pelvic floor disorder is urethriocele.

Women are routinely given supplemental estrogen following hysterectomy/oophorectomy. Many women develop and suffer symptoms of testosterone deficiency that go unrecognized and untreated. Testosterone supplemental therapy for women following hysterectomy/oophorectomy not only can improve the quality of their lives in terms of sexual libido, sexual pleasure, and sense of well-being but also can, as does supplementary estrogen, contribute to the prevention of osteoporosis and urinary incontinence. SARMs can provide androgen replacement in women following hysterectomy/oophorectomy without the hepatotoxic or virilizing side effects of testosterone and other steroidal androgens.

In one embodiment, this invention provides a method for increasing androgen levels in post-hysterectomy and post-oophorectomy women; comprising administering a SARM compound of this invention or its optical isomer, acceptable salt, hydrate, or any combination thereof. In one embodiment, this invention provides a method for treating, preventing, suppressing or inhibiting urinary incontinence in post-hysterectomy and post-oophorectomy women.

In another embodiment, the methods of this invention comprise administering a SARM compound of this invention to a post menopausal, post-hysterectomy, post-oophorectomy women or a combination thereof. Each represent a separate embodiment of this invention.

In one embodiment, the methods of this invention comprise administering a SARM compound of this invention in combination with physiotherapy for SUI. In another embodiment, the methods of this invention comprise administering a SARM compound in combination with surgeries for SUI. In another embodiment, the methods of this invention comprise administering a SARM compound in combination with urinary slings and other medical devices for SUI.

Selective Androgen Receptor Modulator (SARM) Compounds

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) treating, preventing, suppressing or inhibiting stress urinary incontinence, urge urinary incontinence or a combination thereof in a postmenopausal female subject; (k) improving the urethral pressure profile of a subject suffering from SUI; and (l) improving the urethral closure pressure of a subject suffering from SUI is a SARM compound of Formula I, and/or its analog, derivative, isomer, metabolite, pharmaceutically acceptable salt, pharmaceutical product, hydrate, N-oxide, crystal, polymorph, prodrug or any combination thereof:

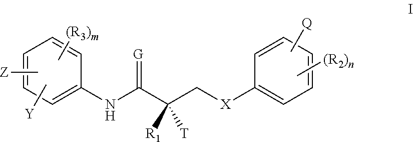

X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is H, F, Cl, Br, I, CH₃, CF₃, OH, CN, NO₂, NHCOCH₃, NHCOCF₃, NHCOR, alkyl, arylalkyl, OR, NH₂, NHR, N(R)₂, or SR;
R₃ is H, F, Cl, Br, I, CN, NO₂, COR, COOH, CONHR, CF₃, Sn(R)₃, or R₃ together with the benzene ring to which it is attached forms a fused ring system represented by the structure:

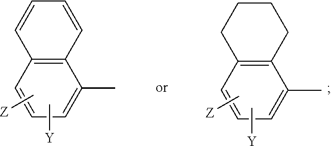

Z is NO₂, CN, COR, COOH, or CONHR;
Y is CF₃, F, Br, Cl, I, CN, or Sn(R)₃;
Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system represented by structure A, B or C:

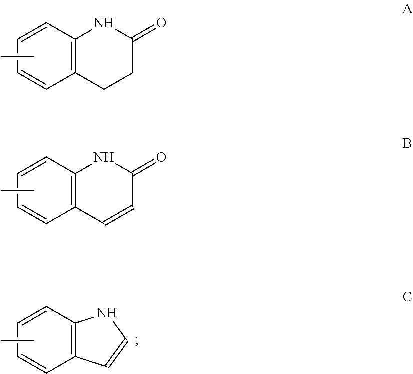

n is an integer of 1-4; and
m is an integer of 1-3.

In one embodiment, G in Formula I is O. In another embodiment, X in Formula I is O. In another embodiment, T in Formula I is OH. In another embodiment, R₁ in Formula I is CH₃. In another embodiment, Z in Formula I is NO₂. In another embodiment, Z in Formula I is CN. In another embodiment, Y in Formula I is CF₃. In another embodiment, Y in Formula I is Cl. In another embodiment, Q in Formula I is CN. In another embodiment, Q in Formula I is halogen. In another embodiment, Q in Formula I is F. In another embodiment, Q in Formula I is Cl. In another embodiment, Q in Formula I is NHCOCH₃. In another embodiment, Q in Formula I is CN and R₂ is F. In another embodiment, Q in Formula I is Cl and R₂ is F. In another embodiment, Q in Formula I is in the para position. In another embodiment, Z in Formula I is in the para position. In another embodiment, Y in Formula I is in the meta position. In one embodiment, the substituent Q is in the para position of the B ring and the substituent R₂ is in the meta position of the B ring.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substituent Q is in the para position of the B ring. In another embodiment, the substituent R₂ is in the meta position of the B ring. In another embodiment, the substituent Q is CN and is in the para position of the B ring.

As contemplated herein, when the integers m and n are greater than one, the substituents R₂ and R₃ are not limited to one particular substituent, and can be any combination of the substituents listed above.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound of Formula IA, and/or its analog, derivative, isomer, metabolite, pharmaceutically acceptable salt, pharmaceutical product, hydrate, N-oxide, crystal, polymorph, prodrug or any combination thereof:

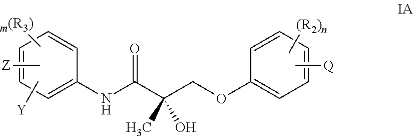

wherein
R₂ is H, F, Cl, Br, I, CH₃, CF₃, OH, CN, NO₂, NHCOCH₃, NHCOCF₃, NHCOR, alkyl, arylalkyl, OR, NH₂, NHR, N(R)₂, or SR;
R₃ is H, F, Cl, Br, I, CN, NO₂, COR, COOH, CONHR, CF₃, Sn(R)₃, or R₃ together with the benzene ring to which it is attached forms a fused ring system represented by the structure:

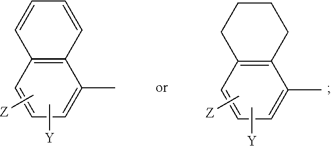

R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or Sn(R)₃;
Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system represented by structure A, B or C:

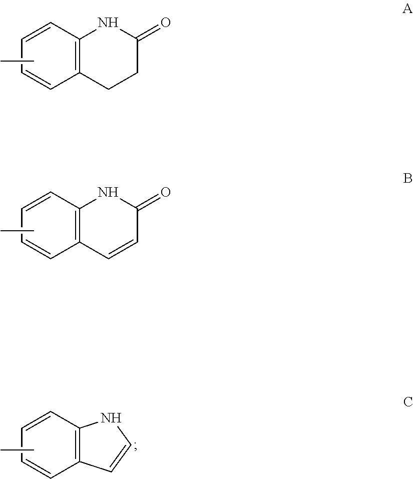

n is an integer of 1-4; and
m is an integer of 1-3.

In another embodiment, Z in Formula IA is NO₂. In another embodiment, Z in Formula IA is CN. In another embodiment, Y in Formula IA is CF₃. In another embodiment, Y in Formula IA is Cl. In another embodiment, Q in Formula IA is CN. In another embodiment, Q in Formula IA is halogen. In another embodiment, Q in Formula IA is F. In another embodiment, Q in Formula IA is Cl. In another embodiment, Q in Formula IA is NHCOCH₃. In another embodiment, Q in Formula IA is CN and R₂ is F. In another embodiment, Q in Formula IA is Cl and R₂ is F. In another embodiment, Q in Formula IA is in the para position. In another embodiment, Z in Formula IA is in the para position. In another embodiment, Y in Formula IA is in the meta position.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substituent Q is in the para position of the B ring. In another embodiment, the substituent R₂ is in the meta position of the B ring. In one embodiment, the substituent Q is in the para position of the B ring and the substituent R₂ is in the meta position of the B ring. In another embodiment, the substituent Q is CN and is in the para position of the B ring.

As contemplated herein, when the integers m and n are greater than one, the substituents R₂ and R₃ are not limited to one particular substituent, and can be any combination of the substituents listed above.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound of Formula II, and/or its analog, derivative, isomer, metabolite, pharmaceutically acceptable salt, pharmaceutical product, hydrate, N-oxide, crystal, polymorph, prodrug or any combination thereof:

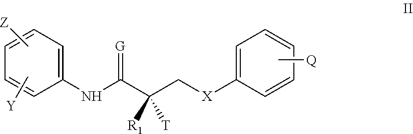

wherein X is a bond, O, CH₂, NH, Se, PR, or NR;
G is O or S;
T is OH, OR, —NHCOCH₃, or NHCOR;
Z is NO₂, CN, COR, COOH or CONHR;
Y is I, CF₃, Br, Cl, or Sn(R)₃;
Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system
represented by structure A, B or C:

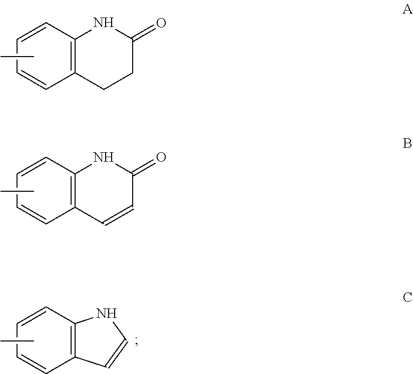

R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CF₃, CH₂CH₃, or CF₂CF₃.

In one embodiment, G in Formula II is O. In another embodiment, X in Formula II is O. In another embodiment, T in Formula II is OH. In another embodiment, R₁ in Formula II is CH₃. In another embodiment, Z in Formula II is NO₂. In another embodiment, Z in Formula II is CN. In another embodiment, Y in Formula II is CF₃. In another embodiment, Y in Formula II is halogen. In another embodiment, Y in Formula II is Cl. In another embodiment, Q in Formula II is CN. In another embodiment, Q in Formula II is halogen. In another embodiment, Q in Formula II is Cl. In another embodiment, Q in Formula II is F. In another embodiment, Q in Formula II is NHCOCH₃. In another embodiment, Q in Formula II is in the para position. In another embodiment, Z in Formula II is in the para position. In another embodiment, Y in Formula II is in the meta position. In another embodiment, G in Formula II is O, T is OH, R₁ is CH₃, X is O, Z is CN, Y is CF₃ or halogen and Q is CN, F, or Cl. In another embodiment, G in Formula II is O, T is OH, R₁ is CH₃, X is O, Z is NO₂, Y is CF₃ and Q is NHCOCH₃, F or Cl.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is CN and is in the para position of the B ring.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound of Formula IIA, and/or its analog, derivative, isomer, metabolite, pharmaceutically acceptable salt, pharmaceutical product, hydrate, N-oxide, crystal, polymorph, prodrug or any combination thereof:

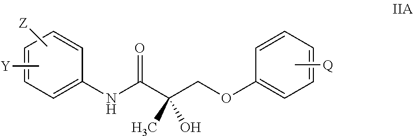

wherein Z is NO₂, CN, COR, COOH or CONHR;
Y is I, CF₃, Br, Cl, or Sn(R)₃;
Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system
represented by structure A, B or C:

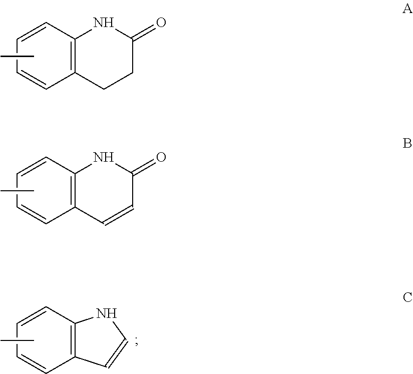

and
R is alkyl, haloalkyl, dihaloalkyl, trihaloalkyl, CH₂F, CHF₂, CF₃, CF₂CF₃, aryl, phenyl, halogen, alkenyl or OH.

In another embodiment, Z in Formula IIA is NO₂. In another embodiment, Z in Formula IIA is CN. In another embodiment, Y in Formula IIA is CF₃. In another embodiment, Y in Formula IIA is halogen. In another embodiment, Y in Formula IIA is Cl. In another embodiment, Q in Formula IIA is CN. In another embodiment, Q in Formula IIA is halogen. In another embodiment, Q in Formula IIA is Cl. In another embodiment, Q in Formula IIA is F. In another embodiment, Q in Formula IIA is NHCOCH₃. In another embodiment, Q in Formula IIA is in the para position. In another embodiment, Z in Formula IIA is in the para position. In another embodiment, Y in Formula IIA is in the meta position.

The substituents Z and Y can be in any position of the ring carrying these substituents (hereinafter “A ring”). In one embodiment, the substituents (hereinafter “A ring”). In one embodiment, Z is in the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is CN and is in the para position of the B ring.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound of Formula III, and/or its analog, derivative, isomer, metabolite, pharmaceutically acceptable salt, pharmaceutical product, hydrate, N-oxide, crystal, polymorph, prodrug or any combination thereof:

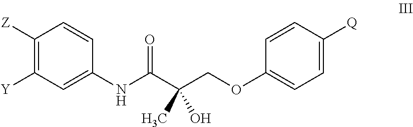

wherein
Z is NO₂, CN, COOH, COR, NHCOR or CONHR;
Y is CF₃, F, I, Br, Cl, CN, C(R)₃ or Sn(R)₃;
Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system represented by structure A, B or C:

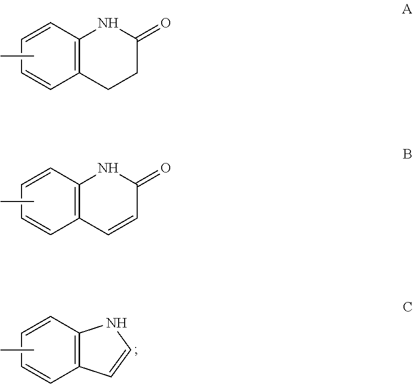

and
R is alkyl, haloalkyl, dihaloalkyl, trihaloalkyl, CH₂F, CHF₂, CF₃, CF₂CF₃, aryl, phenyl, halogen, alkenyl or OH.

In one embodiment, Z in Formula III is NO₂. In another embodiment, Z in Formula III is CN. In another embodiment, Y in Formula III is CF₃. In another embodiment, Y in Formula III is Cl. In another embodiment, Y in Formula III is halogen. In another embodiment, Q in Formula III is CN. In another embodiment, Q in Formula III is halogen. In another embodiment, Q in Formula III is F. In another embodiment, Q in Formula III is Cl. In another embodiment, Q in Formula III is NHCOCH₃. In another embodiment, Z is CN, Y is CF₃ or halogen, and Q is CN, F, or Cl. In another embodiment, Z is NO₂, Y is CF₃, and Q is NHCOCH₃, F or Cl.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound of Formula IV, and/or its analog, derivative, isomer, metabolite, pharmaceutically acceptable salt, pharmaceutical product, hydrate, N-oxide, crystal, polymorph, prodrug or any combination thereof:

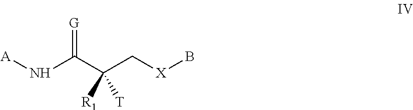

wherein X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from:

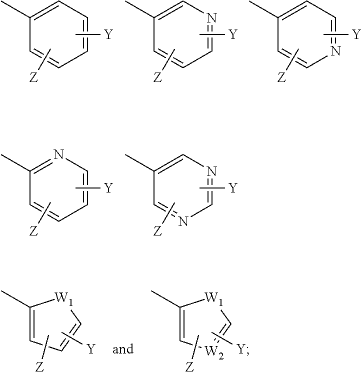

B is a ring selected from:

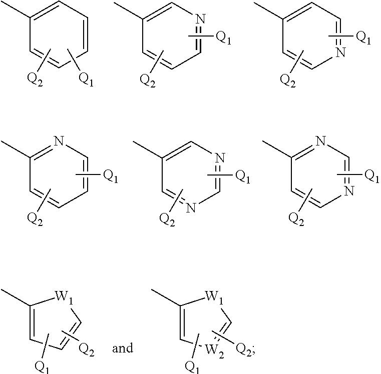

Z is NO₂, CN, COOH, COR, NHCOR or CONHR;
Y is CF₃, F, I, Br, Cl, CN, C(R)₃ or Sn(R)₃;
Q₁ and Q₂ are independently hydrogen, alkyl, halogen, CF₃, CN, C(R)₃, Sn(R)₃, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R, or SR, or

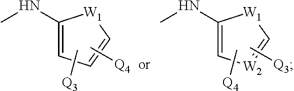

Q₃ and Q₄ are independently of each other a hydrogen, alkyl, halogen, CF₃, CN, C(R)₃, Sn(R)₃, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
   W₁ is O, NH, NR, NO or S; and
   W₂ is N or NO.

In one embodiment, G in Formula IV is O. In another embodiment, X in Formula IV is O. In another embodiment, T in Formula IV is OH. In another embodiment, R₁ in Formula IV is CH₃. In another embodiment, Z in Formula IV is NO₂. In another embodiment, Z in Formula IV is CN. In another embodiment, Y in Formula IV is CF₃. In another embodiment, Y in Formula IV is halogen. In another embodiment, Y in Formula IV is Cl. In another embodiment, Q₁ in Formula II is CN. In another embodiment, Q₁ in Formula IV is F. In another embodiment, Q₁ in Formula IV is Cl. In another embodiment, Q₁ in Formula IV is NHCOCH₃. In another embodiment, Q₁ in Formula IV is in the para position. In another embodiment, Z in Formula IV is in the para position. In another embodiment, Y in Formula IV is in the meta position. In another embodiment, G in Formula IV is O, T is OH, R₁ is CH₃, X is O, Z is NO₂ or CN, Y is CF₃ or halogen and Q₁ is CN, F, Cl, or NHCOCH₃.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substituent Q₁ is in the para position of the B ring. In another embodiment, the substituent is Q₂ is H. In another embodiment, the substituent Q₁ is in the para position of the B ring and the substituent is Q₂ is H. In another embodiment, the substituent Q₁ is CN and is in the para position of the B ring, and the substituent is Q₂ is H, Cl, or F.

In another embodiment, the A ring and the B ring cannot simultaneously be a benzene ring.

As contemplated herein, other specific embodiments of compounds included within the scope of the present invention, and which are useful in: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; are SARM compounds of Formulas V or VI, and/or its analog, derivative, isomer, metabolite, pharmaceutically acceptable salt, pharmaceutical product, hydrate, N-oxide, crystal, polymorph, prodrug or any combination thereof:

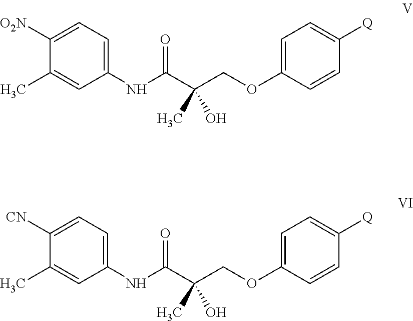

wherein Q is CN, alkyl, halogen, N(R)₂, NHCOCH₃, NHCOCF₃, NHCOR, NHCONHR, NHCOOR, OCONHR, CONHR, NHCSCH₃, NHCSCF₃, NHCSR, NHSO₂CH₃, NHSO₂R, OR, COR, OCOR, OSO₂R, SO₂R or SR;
or Q together with the benzene ring to which it is attached is a fused ring system represented by structure A, B or C:

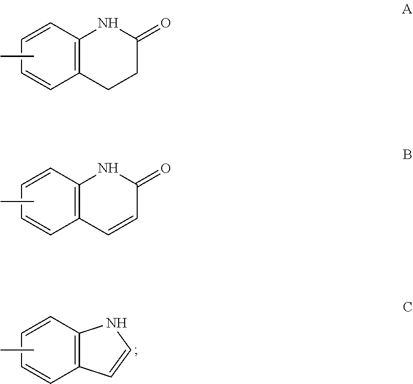

and
R is alkyl, haloalkyl, dihaloalkyl, trihaloalkyl, CH₂F, CHF₂, CF₃, CF₂CF₃, aryl, phenyl, halogen, alkenyl or OH.

In one embodiment, Q in Formulas V or VI is CN. In one embodiment, Q in Formulas V or VI is halogen. In one embodiment, Q in Formulas V or VI is F. In one embodiment, Q in Formulas V or VI is Cl. In one embodiment, Q in Formulas V or VI is NHCOCH₃.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula VII, and/or its analog, derivative, isomer, metabolite, pharmaceutically acceptable salt, pharmaceutical product, hydrate, N-oxide, crystal, polymorph, prodrug or any combination thereof:

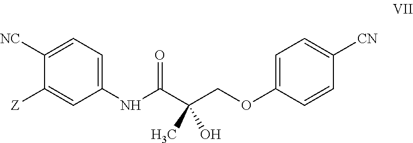

wherein Z is Cl or CF₃.

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula VIII, and/or its analog, derivative, isomer, metabolite, pharmaceutically acceptable salt, pharmaceutical product, hydrate, N-oxide, crystal, polymorph, prodrug or any combination thereof:

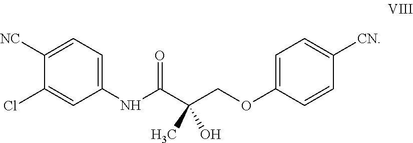 [SMILES: C[C@](O)(COc1ccc(C#N)cc1)C(=O)Nc1ccc(C#N)c(Cl)c1]

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula IX, and/or its analog, derivative, isomer, metabolite, pharmaceutically acceptable salt, pharmaceutical product, hydrate, N-oxide, crystal, polymorph, prodrug or any combination thereof:

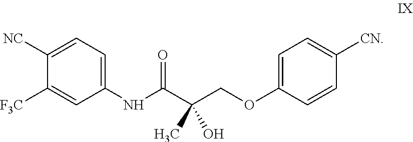 [SMILES: C[C@](O)(COc1ccc(C#N)cc1)C(=O)Nc1ccc(C#N)c(C(F)(F)F)c1]

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula X, and/or its analog, derivative, isomer, metabolite, pharmaceutically acceptable salt, pharmaceutical product, hydrate, N-oxide, crystal, polymorph, prodrug or any combination thereof:

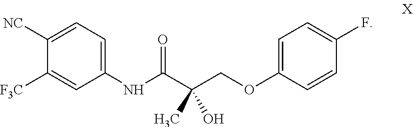 [SMILES: C[C@](O)(COc1ccc(F)cc1)C(=O)Nc1ccc(C#N)c(C(F)(F)F)c1]

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula XI, and/or its analog, derivative, isomer, metabolite, pharmaceutically acceptable salt, pharmaceutical product, hydrate, N-oxide, crystal, polymorph, prodrug or any combination thereof:

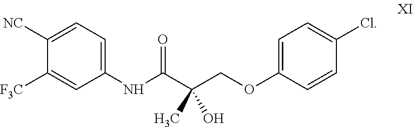 [SMILES: C[C@](O)(COc1ccc(Cl)cc1)C(=O)Nc1ccc(C#N)c(C(F)(F)F)c1]

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula XII, and/or its analog, derivative, isomer, metabolite, pharmaceutically acceptable salt, pharmaceutical product, hydrate, N-oxide, crystal, polymorph, prodrug or any combination thereof:

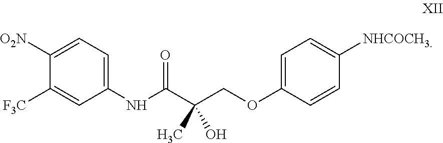

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic-floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula XIII, and/or its analog, derivative, isomer, metabolite, pharmaceutically acceptable salt, pharmaceutical product, hydrate, N-oxide, crystal, polymorph, prodrug or any combination thereof:

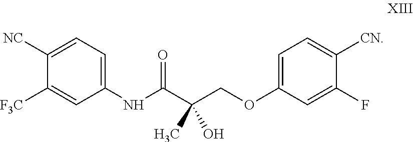 [SMILES: C[C@](O)(COc1ccc(C#N)c(F)c1)C(=O)Nc1ccc(C#N)c(C(F)(F)F)c1]

In one embodiment, the compound of this invention which is effective at: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; is a SARM compound represented by a structure of Formula XIV, and/or its analog, derivative, isomer, metabolite, pharmaceutically acceptable salt, pharmaceutical product, hydrate, N-oxide, crystal, polymorph, prodrug or any combination thereof:

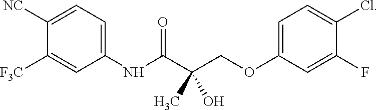 [SMILES: C[C@](O)(COc1ccc(Cl)c(F)c1)C(=O)Nc1ccc(C#N)c(C(F)(F)F)c1]

In one embodiment, the methods of the present invention comprise administering an analog of the compound of Formulas I, IA, II, IIA, III, IV, V, VI, VII, VIII, IX, X, XI, XII, XIII and/or XIV (I-XIV). In another embodiment, the methods of the present invention comprise administering a derivative of the compound of Formulas I-XIV. In another embodiment, the methods of the present invention comprise administering an isomer of the compound of Formulas I-XIV. In another embodiment, the methods of the present invention comprise administering a metabolite of the compound of Formulas I-XIV. In another embodiment, the methods of the present invention comprise administering a pharmaceutically acceptable salt of the compound of Formulas I-XIV. In another embodiment, the methods of the present invention comprise administering a pharmaceutical product of the compound of Formulas I-XIV. In another embodiment, the methods of the present invention comprise administering a hydrate of the compound of Formulas I-XIV. In another embodiment, the methods of the present invention comprise administering an N-oxide of the compound of Formulas I-XIV. In another embodiment, the methods of the present invention comprise administering a polymorph of the compound of Formulas I-XIV. In another embodiment, the methods of the present invention comprise administering a crystal of the compound of Formulas I-XIV. In another embodiment, the methods of the present invention comprise administering a prodrug of the compound of Formulas I-XIV. In another embodiment, the methods of the present invention comprise administering a combination of any of an analog, derivative, metabolite, isomer, pharmaceutically acceptable salt, pharmaceutical product, hydrate, N-oxide, polymorph, crystal or prodrug of the compound of Formulas I-XIV.

In one embodiment, the methods of this invention comprise administering a compound of Formulas I-XIV. In another embodiment, the methods of this invention comprise administering a compound of Formula I. In another embodiment, the methods of this invention comprise administering a compound of Formula IA. In another embodiment, the methods of this invention comprise administering a compound of Formula II. In another embodiment, the methods of this invention comprise administering a compound of Formula IIA. In another embodiment, the methods of this invention comprise administering a compound of Formula III. In another embodiment, the methods of this invention comprise administering a compound of Formula IV. In another embodiment, the methods of this invention comprise administering a compound of Formula V. In another embodiment, the methods of this invention comprise administering a compound of Formula VI. In another embodiment, the methods of this invention comprise administering a compound of Formula VII. In another embodiment, the methods of this invention comprise administering a compound of Formula VIII. In another embodiment, the methods of this invention comprise administering a compound of Formula IX. In another embodiment, the methods of this invention comprise administering a compound of Formula X. In another embodiment, the methods of this invention comprise administering a compound of Formula XI. In another embodiment, the methods of this invention comprise administering a compound of Formula XII. In another embodiment, the methods of this invention comprise administering a compound of Formula XIII. In another embodiment, the methods of this invention comprise administering a compound of Formula XIV.

The compounds of the present invention, either alone or as a pharmaceutical composition, are useful for: (a) treating, preventing, suppressing or inhibiting urological disorders; (b) treating, preventing, suppressing or inhibiting urinary incontinence (UI); (c) treating, preventing, suppressing or inhibiting pelvic floor disorders; and/or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes.

In one embodiment, this invention relates to the treatment of urological disorders. Accordingly, this invention provides methods of: (a) treating, preventing, suppressing or inhibiting urinary incontinence (UI); (b) treating, preventing, suppressing or inhibiting pelvic floor disorders; and/or (c) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; by administering to the subject a therapeutically effective amount of a selective androgen receptor modulator of Formulas I-XIV of this invention, and/or its analog, derivative, isomer, metabolite, pharmaceutically acceptable salt, pharmaceutical product, hydrate, N-oxide, crystal, polymorph, prodrug or any combination thereof, as described herein.

As defined herein, the term “isomer” includes, but is not limited to, optical isomers and analogs, structural isomers and analogs, conformational isomers and analogs, and the like. As used herein, the term “isomer” may also be referred to herein as an “enantiomer” having all of the qualities and properties of an “isomer”.

In one embodiment, this invention encompasses the use of various optical isomers of the selective androgen receptor modulator. It will be appreciated by those skilled in the art that the selective androgen receptor modulators of the present invention contain at least one chiral center. Accordingly, the selective androgen receptor modulators used in the methods of the present invention may exist in, and be isolated in, optically-active or racemic forms. Some compounds may also exhibit polymorphism. It is to be understood that the present invention encompasses any racemic, optically-active, polymorphic, or stereoisomeric form, or any combination thereof, which form possesses properties useful in the treatment of androgen-related conditions described herein. In one embodiment, the selective androgen receptor modulators are the pure (R)-isomers. In another embodiment, the selective androgen receptor modulators are the pure (S)-isomers. In another embodiment, the selective androgen receptor modulators are a mixture of the (R) and the (S) isomers. In another embodiment, the selective androgen receptor modulators are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes “pharmaceutically acceptable salts” of the compounds of this invention, which may be produced, by reaction of a compound of this invention with an acid or base.

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

Suitable pharmaceutically acceptable salts of the compounds of Formulas I-XIV may be prepared from an inorganic acid or from an organic acid. In one embodiment, examples of inorganic salts of the compounds of this invention are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, examples of organic salts of the compounds of this invention may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamates, heptanoates, hexanoates, hydroxymaleates, hydroxycarboxlic acids, hexylresorcinates, hydroxybenzoates, hydroxynaphthoate, hydrofluorate, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In one embodiment, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

This invention further includes derivatives of the selective androgen receptor modulators. The term “derivatives” includes but is not limited to ether derivatives, acid derivatives, amide derivatives, ester derivatives and the like. In addition, this invention further includes hydrates of the selective androgen receptor modulators. The term “hydrate” includes but is not limited to hemihydrate, monohydrate, dihydrate, trihydrate and the like.

This invention further includes metabolites of the selective androgen receptor modulators. The term “metabolite” means any substance produced from another substance by metabolism or a metabolic process.

This invention further includes pharmaceutical products of the selective androgen receptor modulators. The term “pharmaceutical product” means a composition suitable for pharmaceutical use (pharmaceutical composition), as defined herein.

This invention further includes prodrugs of the selective androgen receptor modulators. The term “prodrug” means a substance which can be converted in vivo into a biologically active agent by such reactions as hydrolysis, esterification, de-esterification, activation, salt formation and the like.

This invention further includes crystals of the selective androgen receptor modulators. Furthermore, this invention provides polymorphs of the selective androgen receptor modulators. The term “crystal” means a substance in a crystalline state. The term “polymorph” refers to a particular crystalline state of a substance, having particular physical properties such as X-ray diffraction, IR spectra, melting point, and the like.

In one embodiment of the present invention is a method of: (a) treating, preventing, suppressing or inhibiting urology disorders in a subject; (b) treating, preventing, suppressing or inhibiting urinary incontinence (UI) in a subject; (c) treating, preventing, suppressing or inhibiting pelvic floor disorders in a subject; (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI; comprising the step of administering to the subject a selective androgen receptor modulator of Formulas I-XIV of this invention and/or its analog, derivative, isomer, metabolite, pharmaceutically acceptable salt, pharmaceutical product, hydrate, N-oxide, crystal, polymorph, prodrug or any combination thereof. In one embodiment, the subject is a female subject. In another embodiment, the subject is a male subject.

The substituent R is defined herein as an alkyl, haloalkyl, dihaloalkyl, trihaloalkyl, CH₂F, CHF₂, CF₃, CF₂CF₃, aryl, phenyl, halogen, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond. A halo group refers to F, Cl, Br or I.

An “arylalkyl” or “aralkyl” group refers to an alkyl bound to an aryl, wherein alkyl and aryl are as defined above. An example of an aralkyl group is a benzyl group.

### Biological Activity of Selective Androgen Receptor Modulators

The selective androgen receptor modulators provided herein are a new class of compounds, having anabolic activity, especially in levator ani muscle, which is a pelvic floor muscle. Since treating urinary incontinence involves increasing muscle strength, the SARMs are used herein for treating pelvic floor disorders and specifically UI. The compounds of this invention have a tissue-selective myoanabolic activity profile of a nonsteroidal ligand for the androgen receptor. Furthermore compounds of the present invention are non-aromatizable, non-virilizing, and are not commonly cross-reactive with ER and PR.

As contemplated herein, the appropriately substituted selective androgen receptor modulators of the present invention are useful for: (a) treating, preventing, suppressing or inhibiting urology disorders in a subject; (b) treating, preventing, suppressing or inhibiting urinary incontinence (UI) in a subject; (c) treating, preventing, suppressing or inhibiting pelvic floor disorders in a subject; or (d) reducing the occurrence or lessening the severity of at least one of the following symptoms in a subject suffering from urinary incontinence: (i) average daily frequency of urination; (ii) average nightly frequency of urination; (iii) total urinary incontinence episodes; (iv) stress incontinence episodes; and (v) urinary urgency episodes; (e) providing androgen replacement therapy in post-hysterectomy and post-oophorectomy women; (f) treating, preventing, suppressing or inhibiting urinary incontinence in post-hysterectomy and post-oophorectomy women; (g) treating, preventing, suppressing or inhibiting fecal incontinence; (h) increasing the size and/or weight of muscles in the pelvic floor; (i) increasing the size/strength of the urethral sphincter; (j) improving the urethral pressure profile of a subject suffering from SUI; (k) treating, preventing, suppressing or inhibiting stress urinary incontinence, urge urinary incontinence or a combination thereof in a postmenopausal female subject; and (l) improving the urethral closure pressure of a subject suffering from SUI.

In one embodiment, this invention provides a method of treating, preventing, suppressing or inhibiting stress urinary incontinence, urge urinary incontinence or a combination thereof in a postmenopausal female subject, comprising administering to said subject a composition comprising Compound IX or its isomer, hydrate, pharmaceutically acceptable salt, pharmaceutical composition or any combination thereof. In another embodiment, the composition is administered at a daily dose of between 1 to 3 mg.

In one embodiment, this invention provides a method of treating, preventing, suppressing or inhibiting stress urinary incontinence, urge urinary incontinence or a combination thereof in a postmenopausal female subject, comprising administering to said subject a composition comprising Compound IX or its isomer, hydrate, pharmaceutically acceptable salt, pharmaceutical composition or any combination thereof. In another embodiment, the composition is administered at a daily dose of between 1 to 3 mg.

In one embodiment this invention provides a method of reducing the occurrence or lessening the severity of at least one of the following symptoms in a postmenopausal female subject suffering from stress urinary incontinence, urge urinary incontinence or a combination thereof: (i) average daily frequency of urination; (ii) average nightly frequency of urination; (iii) total urinary incontinence episodes; (iv) stress incontinence episodes; and (v) urinary urgency episodes; comprising administering Compound IX or its isomer, hydrate, pharmaceutically acceptable salt, pharmaceutical composition or any combination thereof. In another embodiment, the composition is administered at a daily dose of between 1 to 3 mg.

In some embodiments, the subject suffers from stress urinary incontinence and urge urinary incontinence.

In some embodiments, the methods of this invention comprise administering a composition comprising Compound IX at a daily dose of between 1 to 3 mg. In other embodiments, the daily dose is 1 mg, 2 mg or 3 mg. Each represents a separate embodiment of this invention. In other embodiments, the administering of the composition of compound IX is up to 12 weeks. In other embodiments, the administering step is by intermittent dosing cycles. In other embodiments, the administering step is by one dose cycle only regimen of up to 12 weeks. In other embodiments, the duration of response following completion of the treatment is at least seven months post-treatment.

The urethra in the female is approximately 4 cm long (compared to 22 cm long in the male). It is imbedded in the connective tissue supporting the anterior vagina. The urethra is composed of an inner epithelial lining, a spongy submucosa, a middle smooth muscle layer, and an outer fibroelastic connective-tissue layer. The spongy submucosa contains a rich vascular plexus that is responsible, in part, for providing adequate urethral occlusive pressure. Urethral smooth muscle and fibroelastic connective tissues circumferentially augment the occlusive pressure generated by the submucosa. Thus, all structural components of the urethra, including the striated sphincter muscle discussed later, contribute to its ability to coapt and prevent urine leakage.

The female urethra is composed of 4 separate tissue layers that keep it closed. The inner mucosal lining keeps the urothelium moist and the urethra supple. The vascular spongy coat produces the mucus important in the mucosal seal mechanism. Compression from the middle muscular coat helps to maintain the resting urethral closure mechanism. The outer seromuscular layer augments the closure pressure provided by the muscular layer.

The smooth muscle of the urethra is arranged longitudinally and obliquely with only a few circular fibers. The nerve supply is cholinergic and alpha-adrenergic. The longitudinal muscles may contribute to shortening and opening of the urethra during voiding. The oblique and circular fibers contribute to urethral closure at rest.

The striated urethral musculature is complex. Its components and their orientation are not agreed upon universally. The voluntary urethral sphincter actually is a group of circular muscle fibers and muscular loops within the pelvic floor. The innermost layer, which is prominent in the proximal two thirds of the urethra, is the sphincter urethrae. More distally, the compressor urethrae and urethrovaginal sphincter are predominant.

These 2 muscles emanate from the anterolateral aspect of the distal half to distal third of the urethra and arch over its anterior or ventral surface. These striated muscles function as a unit. Because they are composed primarily of slow-twitch muscle fibers, these muscles serve ideally to maintain resting urethral closure. The muscles probably do maintain resting urethral closure, but they are known specifically to contribute to voluntary closure and reflex closure of the urethra during acute instances (e.g., coughing, sneezing, laughing) of increased intra-abdominal pressure. The medial pubovisceral portion of the levator ani complex also is a major contributor to active bladder neck and urethral closure in similar situations.

The posterior wall of the urethra is embedded in and supported by the endopelvic connective tissue. The endopelvic connective tissue in this area is attached to the perineal membrane ventrally and laterally to the levator ani muscles by way of the arcus tendinous fascia pelvis. The arcus tendinous fascia pelvis is a condensation of connective tissue, which extends bilaterally from the inferior part of the pubic bone along the junction of the fascia of the obturator internus and levator ani muscle group to near the ischial spine. This tissue provides secondary support to the urethra, bladder neck, and bladder base.

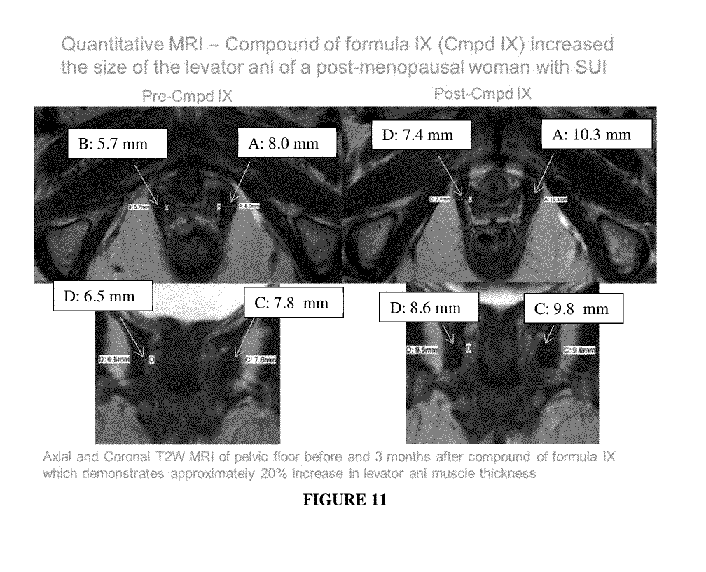

Defects in this tissue are believed to result in cystocele development and urethral hypermobility. The primary support to this area and the entire pelvic floor is believed to be the levator ani muscle complex. At rest, the constant tone mediated by slow-twitch muscle fibers is thought to constitute the major supportive mechanism. Similar to the urethral sphincter muscle groups, the fast-twitch fibers of the levator ani complex aid in suddenly stopping the urinary stream during the voluntary guarding reflex. With acute increases in intra-abdominal pressure, forceful contraction of these fast-twitch levator fibers elevates the pelvic floor and tightens connective-tissue planes, thereby supporting the pelvic viscera. FIG. 11 demonstrates that compound of Formula IX increases the size of the levator ani muscle of a post-menopausal woman with SUI.

Unlike male anatomy, in which the bladder neck and prostate comprise the internal urinary sphincter, the internal sphincter in females is functional rather than anatomic. The bladder neck and proximal urethra constitute the female internal sphincter. However, female external sphincter (i.e., rhabdosphincter) has the most prominent effect on the female urethra.

The female urethra contains an internal sphincter and an external sphincter. The internal sphincter is more of a functional concept than a distinct anatomic entity. The external sphincter is the muscle strengthened by Kegel exercises.

In one embodiment, non-limiting examples of “urology disorder” or “urologic disorder” or “urological disorder” as used herein include urinary incontinence, stress urinary incontinence, psychogenic urinary incontinence, urge urinary incontinence, reflex urinary incontinence, overflow urinary incontinence, neurogenic urinary incontinence, stress urinary incontinence caused by dysfunction of the bladder, overactive/oversensitive bladder, enuresis, nocturia, cystitis, urinary calculi, prostate disorder, kidney disorder, or a urinary tract infection.

In one embodiment, non-limiting examples of a “urinary incontinence” as used herein include stress incontinence, urge incontinence, reflex incontinence, overflow incontinence, neurogenic urinary incontinence, psychogenic incontinence or a combination thereof.

In one embodiment, non-limiting examples of “pelvic floor disorder” as used herein include cystocele, vaginal prolapse, vaginal hernia, rectocele, enterocele, uterocele, and/or urethrocele.

In one embodiment, this invention is directed to a method of treating, preventing, suppressing or inhibiting urology disorders in a subject comprising administering to the subject a therapeutically effective amount of a SARM compound according to this invention. In another embodiment, the urology disorders comprise urinary incontinence, stress urinary incontinence, psychogenic urinary incontinence, urge urinary incontinence, reflex urinary incontinence, overflow urinary incontinence, neurogenic urinary incontinence, stress urinary incontinence caused by dysfunction of the bladder, overactive/oversensitive bladder, enuresis, nocturia, cystitis, urinary calculi, prostate disorder, kidney disorder, a urinary tract infection or any combination thereof. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of treating, preventing, suppressing or inhibiting urinary incontinence (UI) in a subject comprising administering to the subject a therapeutically effective amount of a SARM compound according to this invention. In another embodiment, the urinary incontinence is stress incontinence, urge incontinence, reflex incontinence, overflow incontinence, neurogenic urinary incontinence, psychogenic incontinence or any combination thereof. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of treating, preventing, suppressing or inhibiting stress urinary incontinence (SUI) in a subject comprising administering to the subject a therapeutically effective amount of a SARM compound according to this invention. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

Urge incontinence refers to the involuntary loss of urine that usually occurs when a person has a strong, sudden need to urinate. Stress and urge incontinence often coexist. Approximately half of the women with mixed incontinence (stress and urge) will be relieved of urge incontinence following a procedure for stress incontinence.

In one embodiment, this invention is directed to a method of treating, preventing, suppressing or inhibiting urge urinary incontinence (UUI) in a subject comprising administering to the subject a therapeutically effective amount of a SARM compound according to this invention. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy woman. In another embodiment, the compound is a compound of Formula IX. In another embodiment, the compound is a compound of Formula VIII. In another embodiment, the therapeutically effective amount is between 1 to 3 mg daily. In another embodiment, the therapeutically effective amount is 3 mg daily.

In one embodiment, this invention is directed to a method of treating, preventing, suppressing or inhibiting urge urinary incontinence (UUI) and stress urinary incontinence (SUI) in a subject comprising administering to the subject a therapeutically effective amount of a SARM compound according to this invention. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy woman. In another embodiment, the compound is a compound of Formula IX. In another embodiment, the compound is a compound of Formula VIII. In another embodiment, the therapeutically effective amount is between 1 to 3 mg daily. In another embodiment, the therapeutically effective amount is 3 mg daily.

In one embodiment, this invention is directed to a method of treating, preventing, suppressing or inhibiting pelvic floor disorders in a subject comprising administering to the subject a therapeutically effective amount of a SARM compound according to this invention. In another embodiment, the pelvic floor disorder is cystocele, vaginal prolapse, vaginal hernia, rectocele, enterocele, uterocele, urethrocele or any combination thereof. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of reducing the occurrence or lessening the severity of the symptoms in a subject suffering from urinary incontinence comprising administering to the subject a therapeutically effective amount of a SARM compound according to this invention. In another embodiment, the symptoms are high average daily frequency of urination, high average nightly frequency of urination, urinary incontinence episodes, stress incontinence episodes, urinary urgency episodes or any combination thereof. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of providing androgen replacement therapy in post-hysterectomy and post-oophorectomy women comprising administering to the subject a therapeutically effective amount of a SARM compound according to this invention. In another embodiment, the compound is a compound of Formula IX. In another embodiment, the compound is a compound of Formula VIII. In another embodiment, the therapeutically effective amount is 3 mg daily.

In one embodiment, this invention is directed to a method of treating, preventing, suppressing or inhibiting urinary incontinence in post-hysterectomy and post-oophorectomy women comprising administering to the subject a therapeutically effective amount of a SARM compound according to this invention. In another embodiment, the compound is a compound of Formula IX. In another embodiment, the compound is a compound of Formula VIII. In another embodiment, the therapeutically effective amount is 3 mg daily.

In one embodiment, this invention is directed to a method of treating, preventing, suppressing or inhibiting fecal incontinence in a subject comprising administering to the subject a therapeutically effective amount of a SARM compound according to this invention. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of increasing the size and/or weight of muscles in the pelvic floor of a subject comprising administering to the subject a therapeutically effective amount of a SARM compound according to this invention. In another embodiment, the muscles comprise the levator ani muscles. In another embodiment, the muscles comprise the ischiococcygeus. In another embodiment, the muscles comprise the coccygeus (COC) muscle. In another embodiment, the muscles comprise the pubococcygeus (Pc) muscle. In another embodiment, the muscles comprise the iliococcygeus (IL) muscle. In another embodiment, the muscles comprise the levator ani, ischiococcygeus, coccygeus (COC) muscle, pubococcygeus (Pc), iliococcygeus (IL) or any combination thereof. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

In one embodiment, this invention is directed to a method of increasing the size and/or weight of urethral sphincter of a subject comprising administering to the subject a therapeutically effective amount of a SARM compound according to this invention. In another embodiment, the subject is a female. In another embodiment, the subject is a male. In another embodiment, the subject is a postmenopausal woman. In another embodiment, the subject is a post-hysterectomy woman. In another embodiment, the subject is a post-oophorectomy women. In another embodiment, the compound is a compound of Formula IX. In another embodiment, the compound is a compound of Formula VIII. In another embodiment, the therapeutically effective amount is 3 mg daily.

Steroid hormones are one example of small hydrophobic molecules that diffuse directly across the plasma membrane of target cells and bind to intracellular cell signaling receptors. These receptors are structurally related and constitute the intracellular receptor superfamily (or steroid-hormone receptor superfamily). Steroid hormone receptors include but are not limited to progesterone receptors, estrogen receptors, androgen receptors, glucocorticoid receptors, and mineralocorticoid receptors. In one embodiment, the present invention is directed to androgen receptors. In one embodiment, the present invention is directed to androgen receptor agonists. In one embodiment, the present invention is directed to progesterone receptors. In one embodiment, the present invention is directed to progesterone receptor antagonists.

In addition to ligand binding to the receptors, the receptors can be blocked to prevent ligand binding. When a substance binds to a receptor, the three-dimensional structure of the substance fits into a space created by the three-dimensional structure of the receptor in a ball and socket configuration. The better the ball fits into the socket, the more tightly it is held. This phenomenon is called affinity. If the affinity of a substance is greater than the original hormone, it will compete with the hormone and bind the binding site more frequently. Once bound, signals may be sent through the receptor into the cells, causing the cell to respond in some fashion. This is called activation. On activation, the activated receptor then directly regulates the transcription of specific genes. But the substance and the receptor may have certain attributes, other than affinity, in order to activate the cell. Chemical bonds between atoms of the substance and the atoms of the receptors may form. In some cases, this leads to a change in the configuration of the receptor, which is enough to begin the activation process (called signal transduction).

In one embodiment, a receptor antagonist is a substance which binds receptors and inactivates them. In one embodiment, a selective androgen receptor modulator is a molecule that exhibits in vivo tissue selectivity, activating signaling activity of the androgen receptor (AR) in anabolic (muscle, bone, etc.) tissues to a greater extent than in the androgenic tissues. Thus, in one embodiment, the selective androgen receptor modulators of the present invention are useful in binding to and activating steroidal hormone receptors. In one embodiment, the SARM compound of the present invention is an agonist which binds the androgen receptor. In another embodiment, the compound has high affinity for the androgen receptor.

Assays to determine whether the compounds of the present invention are AR agonists or antagonists are well known to a person skilled in the art. For example, AR agonistic activity can be determined by monitoring the ability of the selective androgen receptor modulators to maintain and/or stimulate the growth of AR containing androgenic tissue such as prostate and seminal vesicles, as measured by weight, in castrated animals. AR antagonistic activity can be determined by monitoring the ability of the selective androgen receptor modulators to inhibit the growth of AR containing tissue in intact animals or counter the effects of testosterone in castrated animals.

An androgen receptor (AR) is an androgen receptor of any species, for example a mammal. In one embodiment, the androgen receptor is an androgen receptor of a human. Thus, in another embodiment, the selective androgen receptor modulators bind reversibly to an androgen receptor of a human. In another embodiment, the selective androgen receptor modulators bind reversibly to an androgen receptor of a mammal.

As contemplated herein, the term “selective androgen receptor modulator” (SARM) refers to, in one embodiment, a molecule that exhibits in vivo tissue selectivity, activating signaling activity of the androgen receptor in anabolic (muscle, bone, etc.) tissues to a greater extent than in the androgenic tissues. In another embodiment, a selective androgen receptor modulator selectively binds the androgen receptor. In another embodiment, a selective androgen receptor modulator selectively affects signaling through the androgen receptor. In one embodiment, the SARM is a partial agonist. In one embodiment, the SARM is a tissue-selective agonist, or in some embodiments, a tissue-selective antagonist.

In one embodiment, a SARM of this invention exerts its effects on the androgen receptor in a tissue-dependent manner. In one embodiment, a SARM of this invention will have an IC₅₀ or EC₅₀ with respect to AR, as determined using AR transactivation assays, as known in the art, or, in other embodiments, as described herein.

As used herein, the term “treating” is disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission. As used herein, the term “delaying” means stopping, hindering, slowing down, postponing, holding up or setting back.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, a compound of the present invention is administered to a subject once a week. In another embodiment, a compound of the present invention is administered to a subject twice a week. In another embodiment, a compound of the present invention is administered to a subject three times a week. In another embodiment, a compound of the present invention is administered to a subject four times a week. In another embodiment, a compound of the present invention is administered to a subject five times a week. In another embodiment, a compound of the present invention is administered to a subject daily. In another embodiment, a compound of the present invention is administered to a subject multiple times daily. In another embodiment, a compound of the present invention is administered to a subject weekly. In another embodiment, a compound of the present invention is administered to a subject bi-weekly. In another embodiment, a compound of the present invention is administered to a subject monthly.

In one embodiment, the methods of the present invention comprise administering a selective androgen receptor modulator as the sole active ingredient. However, also encompassed within the scope of the present invention are methods for treating, preventing, suppressing or inhibiting urology disorders, which comprise administering the selective androgen receptor modulators in combination with one or more therapeutic agents. In one embodiment, the therapeutic agent in combination with the SARM of this invention includes: non-selective anti-cholinergics such as oxybutynin and propantheline, or anti-muscarinics such as tolterodine, trospium, solifenacin, darifenacin, and fesoterodine.

In one embodiment, the therapeutic agent in combination with the SARM of this invention includes: Adrenergic modulators for UI such as tricyclic anti-depressants (e.g., imipramine and amitriptyline) and the 33-adrenergic agonist (e.g., mirabegron).

In one embodiment, the therapeutic agent in combination with the SARM of this invention include: muscle relaxants (e.g., relax the detrusor) such as flavoxate and dicylcomine, or botulinum toxins such as onabotulinumtoxin A.

In one embodiment, the methods of the present invention comprise administering a pharmaceutical composition (or pharmaceutical preparation, used herein interchangeably) comprising the selective androgen receptor modulator of the present invention and/or its analog, derivative, isomer, metabolite, pharmaceutical product, hydrate, N-oxide, polymorph, crystal, prodrug or any combination thereof; and a suitable carrier or diluent.

### Pharmaceutical Compositions

As used herein, “pharmaceutical composition” means therapeutically effective amounts of the selective androgen receptor modulator together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCI, acetate, phosphate), pH and ionic strength, additives such as albumin or gelatin to prevent absorption to surfaces, detergents (e.g., Tween 20®, Tween 80®, Pluronic F68®, bile acid salts), solubilizing agents (e.g., glycerol, polyethylene glycerol), anti-oxidants (e.g., ascorbic acid, sodium metabisulfite), preservatives (e.g., Thimerosal®, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal and oral. In one embodiment, the pharmaceutical composition is administered parenterally, paracancerally, transmucosally, transdermally, intramuscularly, intravenously, intradermally, subcutaneously, intraperitoneally, intraventricularly, intravaginally, intracranially and intratumorally.

Further, as used herein “pharmaceutically acceptable carriers” are well known to those skilled in the art and include, but are not limited to, 0.01-0.1 M and preferably 0.05 M phosphate buffer or about 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's® dextrose, dextrose and sodium chloride, lactated Ringer's® and fixed oils. Intravenous vehicles include fluid and nutrient replenishers, electrolyte replenishers such as those based on Ringer's® dextrose, and the like. Preservatives and other additives may also be present, such as, for example, antimicrobials, antioxidants, collating agents, inert gases and the like.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in proximity to the therapeutic target, e.g., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The pharmaceutical preparation can comprise the selective androgen receptor modulator alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the selective androgen receptor modulator can be administered to a subject by, for example, subcutaneous implantation of a pellet; in a further embodiment, the pellet provides for controlled release of selective androgen receptor modulator over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository.

The pharmaceutical preparations of the invention can be prepared by known dissolving, mixing, granulating, or tablet-forming processes. For oral administration, the selective androgen receptor modulators or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the selective androgen receptor modulators or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions can be prepared as aerosols of the active component delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the selective androgen receptor modulators or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

For use in medicine, the salts of the selective androgen receptor modulator will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

In one embodiment, the term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers.

In some embodiments, the term “comprise” or grammatical forms thereof, refers to the inclusion of the indicated active agent, such as the compound of this invention, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and belong to a class of compounds distinct from that of the indicated active ingredient, by acting via a different mechanism of action, for example, and representing an embodiment of this invention, polypeptides comprising T cell epitopes present in a composition may be specifically combined with polypeptides comprising B cell epitopes. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

Further, as used herein, the term “comprising” is intended to mean that the system includes the recited elements, but not excluding others which may be optional. By the phrase “consisting essentially of” it is meant a method that includes the recited elements but exclude other elements that may have an essential significant effect on the performance of the method. “Consisting of” shall thus mean excluding more than traces of other elements. Embodiments defined by each of these transition terms are within the scope of this invention.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, the term “a” or “one” or “an” refers to at least one. In one embodiment, the phrase “two or more” may be of any denomination, which will suit a particular purpose. In one embodiment, “about” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

The following examples are presented in order to more fully illustrate the preferred embodiments of the invention. They should in no way be construed, however, as limiting the broad scope of the invention.

### EXAMPLES

### Example 1

### Use of SARMs for Increase Muscles of the Pelvic Floor

As discussed above, weakening and/or atrophy of the levator ani can lead to instability of the pelvic floor and inability to maintain urethral closure during transiently elevated intraabdominal pressure, resulting in stress urinary incontinence. The levator ani muscle, and other muscles of the pelvic floor such as the urethral sphincter, are exquisitely sensitive to the anabolic actions of androgens [Hershberger et al., Myotrophic activity of such as testosterone and other steroids as determined by modified levator ani muscle method. [Proc. Soc. Exp. Biol. Med. (1953) 83: 175-180; Ho et al., Anabolic effects of androgens on muscles of female pelvic floor and lower urinary tract. Curr. Opin. Obstet. Gynecol. (2004) 16: 405-409].

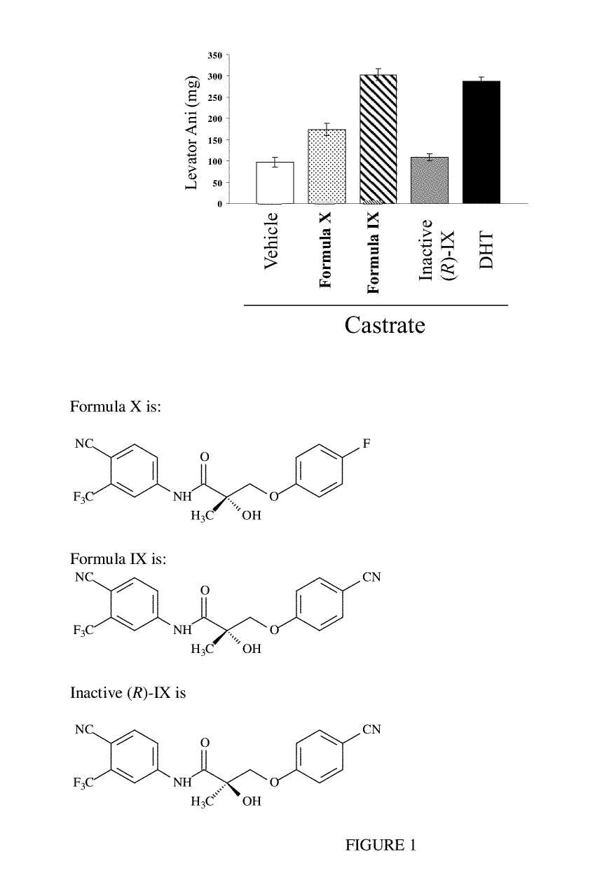

Treatment with DHT or Formula X and Formula IX in vivo elicits hypertrophy of the levator ani muscle as presented in FIG. 1. Sprague Dawley rats (n=5; 200 g weight) that were castrated and treated subcutaneously for 14 days with vehicle (open bars), 3 mg/day Formula X (dotted bars), Formula IX (hatched bars), an inactive propanamide compound (R)-IX (grey bars) and DHT (black bars). At sacrifice, organs were weighed and expressed as raw organ weights. Values are expressed as average±S.D.

AR is prevalent in many structures of the genitourinary system and androgens may have other beneficial effects in maintaining continence or compensating for incontinence. Similarly, urethral smooth muscles also are likely to be strengthened by the use of SARMs.

### Example 2

### Non-Steroidal Tissue-Selective Androgen Receptor Modulators (SARMs) Improve Pelvic Floor Muscle Mass and Architecture in Female Ovariectomized Mice

The androgen receptor (AR) is a ligand-activated transcription factor that is critical for the growth and development of muscle, bone, endocrine and reproductive organs. In the absence of ligand (i.e., endogenous androgens), the AR is maintained in an inactive complex through its interactions with heat shock proteins (HSPs) and corepressors. Upon ligand (e.g., testosterone or dihydrotestosterone) binding, the HSPs dissociate from the AR, leading to a change in its conformation and the subsequent dimerization and nuclear localization of the AR. The AR dimer binds to hormone response elements (HRE) on the promoter of hormone responsive gene, recruits various coactivators and general transcription factors, and induces the transcription of the target gene. Although many tissues have cells that possess ARs and are considered to be androgen responsive, one of the tissues that has the highest concentration of AR is the levator ani muscle. The levator ani muscle, along with other pelvic floor muscles, responds to the presence of androgens and through the AR, these androgens can robustly increase the size and weight of these muscles.

The pelvic floor is composed of striated muscles, which support the bladder, uterus, and rectum. The muscles specific to the pelvic floor include, principally, the levator ani and ischiococcygeus (also known as the coccygeus) which, as mentioned above, are known to contain a relatively high expression of the AR.

The objective of this study is to evaluate the effect of selective androgen receptor modulators (SARMs) on pelvic floor muscle weight and gene expression.

### Materials and Methods:

Six to eight week old female mice (n=5-7) purchased from JAX® Mice were ovariectomized (OVX) or sham operated. Twenty days after OVX, treatment was initiated as outlined in the table below. Compounds of Formulas IX and VIII were dissolved in DSMO/PEG 300 (15:85) and were administered by oral gavage. Body weight and MRI measurements, to evaluate total body lean mass, were recorded at the beginning and at the end of the treatment. The animals were treated for twenty-eight days and then sacrificed, pelvic floor muscles isolated, weighed and stored for RNA and protein extraction. The expression of genes involved in catabolism and anabolism of muscle was measured by mRNA analysis. The serum concentrations of the drugs were measured by LC-MS/MS. The statistical analysis was performed using JMP Pro® software utilizing one way analysis of variance.

| Group No. | Treatment (mg/kg/day) p.o. | | Surgery |
|---|---|---|---|
| 1 | Vehicle | | Intact |
| 2 | Vehicle | | OVX |
| 3 | 0.5 | mg IX | OVX |
| 4 | 2.5 | mg IX | OVX |
| 5 | 5 | mg IX | OVX |
| 6 | 0.5 | mg VIII | OVX |
| 7 | 2.5 | mg VIII | OVX |
| 8 | 5 | mg VIII | OVX |

Gene Expression Studies:

Formalin fixed tissues were homogenized using a FastPrep® tissue homogenizer and RNA was isolated using Qiagen® RNA isolation kit. RNA was quantified and 1 μg RNA from each sample was used to synthesize cDNA using cDNA synthesis kit from Life Technologies®. Realtime rtPCR was performed with Taqman primers and probe from Life Technologies® on an ABI-7900 realtime PCR machine. The expression of various genes was normalized to GAPDH.

Plasma Extraction for Compound of Formula IX and Compound of Formula VIII:

After samples were thawed, a 100 μL aliquot of mouse serum from each sample was mixed with 200 μL acetonitrile containing 200 nM compound of Formula XIII as the internal standard. After each sample was thoroughly vortex for 15 seconds, the sample was centrifuged at 3000 rpm for 10 min. Approximately 200 μL supernatant was transferred for LC-MS/MS analysis

Preparation of the Standard Curve:

Stock solutions of compounds of Formulas IX and VIII were 100 μM in DMSO. A dilution of 1:50 with control mouse serum was made and 200 μL of 2 μM was added to the first micro centrifuge tube. 100 μL of control mouse serum was added to the next 7 micro centrifuge tubes. Transferred 100 μL from tube 1 (2 μM) to tube 2, vortexed and continued the 2 fold dilution through tube 7. 200 μL of acetonitrile containing 200 nM compound of Formula XIII as the internal standard was added to each tube. After vortexing and centrifuging, 200 μL was transferred to LC-MS/MS analysis. The concentration of each standard curve ranged from 1 μM to 0.0078 μM.

LC-MS/MS Analysis:

The analysis of compounds of Formulas IX and VIII in serum was performed using LC-MS/MS system consisting of Agilent 1100 HPLC with an MDS/Sciex 4000 Q-Trap™ mass spectrometer. The separation was achieved using a C₁₈ analytical column (Alltima™, 2.1×100 mm, 3 μm) protected by a C₁₈ guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 mL/min. The total runtime for compound of Formula IX was 4.5 min, and the volume injected was 10 μL. The total runtime for compound of Formula VIII was also 4.5 min, and the volume injected was 10.0 μL. Multiple reaction monitoring (MRM) scans were made with curtain gas at 30 for compound of Formula IX, 25 for compound of Formula VIII; collision gas at medium for compound of Formula IX, high for compound of Formula VIII; nebulizer gas and auxiliary gases at 60 and source temperature at 550° C. for both. Molecular ions were formed using an ion spray voltage (IS) of 4200 V (negative mode). Declustering potential (DP), entrance potential (EP), collision energy (CE), product ion mass, and cell exit potential (CXP) were optimized with the values of −20.0, −10.0, −30.0, and −15.0, respectively, for the mass pair 388.1/118.1 (compound of Formula IX). Declustering potential (DP), entrance potential (EP), collision energy (CE), product ion mass, and cell exit potential (CXP) were optimized with the values of −95.9, −9.94, −40.0, and −15.0, respectively, for the mass pair 354.0/118.1 (compound of Formula VIII).

Histology:

Pelvic floor muscles were paraffin embedded and sections were stained for collagen (Mason trichrome) and elastin (Van Gieson). Stains were evaluated by a pathologist for fiber length and stain intensity.

### Results:

The coccygeus (COC) muscle (located posterior to levator ani) and levator ani (pubococcygeus (Pc) muscle+iliococcygeus (IL) muscle) are two essential elements of the pelvic floor that provide for support and function. The COC and levator ani or LA (Pc+IL) in association with the levator plate support the pelvic floor and form the pelvic diaphragm. The largest of the three muscle types is the COC, followed by the Pc and the IL. In mice, the COC weight is equal to or greater than that of the Pc and IL combined.

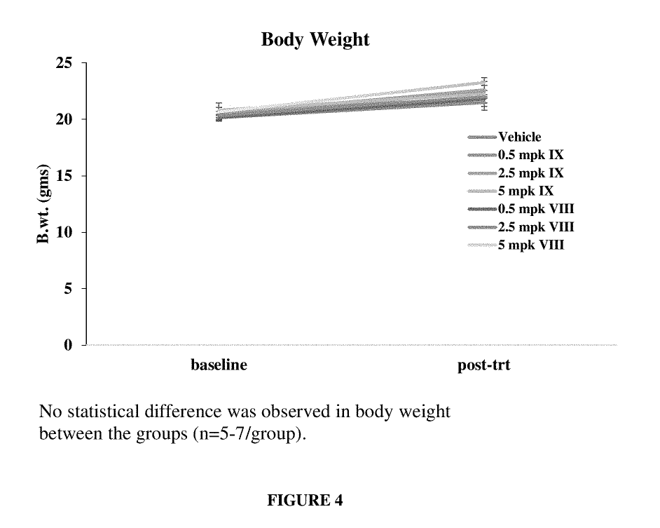

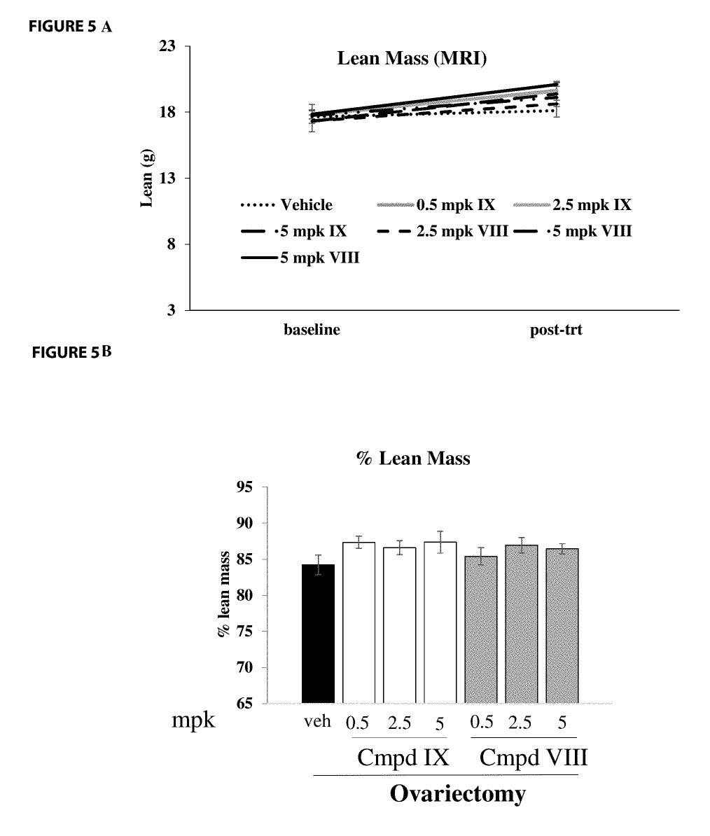

As described above, the objective of these studies was to examine the effect of two SARMs on the pelvic floor muscles. The total body weight of the animals treated with the SARMs increased modestly, although not statistically significantly (FIG. 4). Similarly, MRI measurements demonstrated an increasing trend in the total lean body mass with increasing dose (FIG. 5). However, as was the case with body weight, this trend in lean muscle mass did not attain significance.

The pelvic floor muscles in mice are small making the unmagnified visualization difficult. In order to improve the resolution, the pelvic regions of the mice were immersed in formalin for two days after sacrifice before dissecting them under microscope along with precise weight measurement. The COC, Pc, and IL were isolated and weighed using a microbalance that has a resolution as low as a microgram.

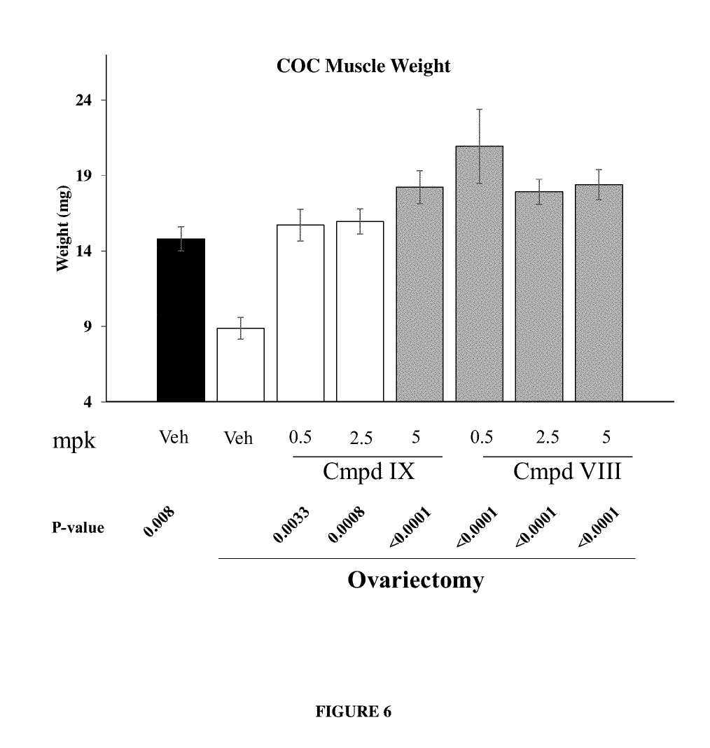

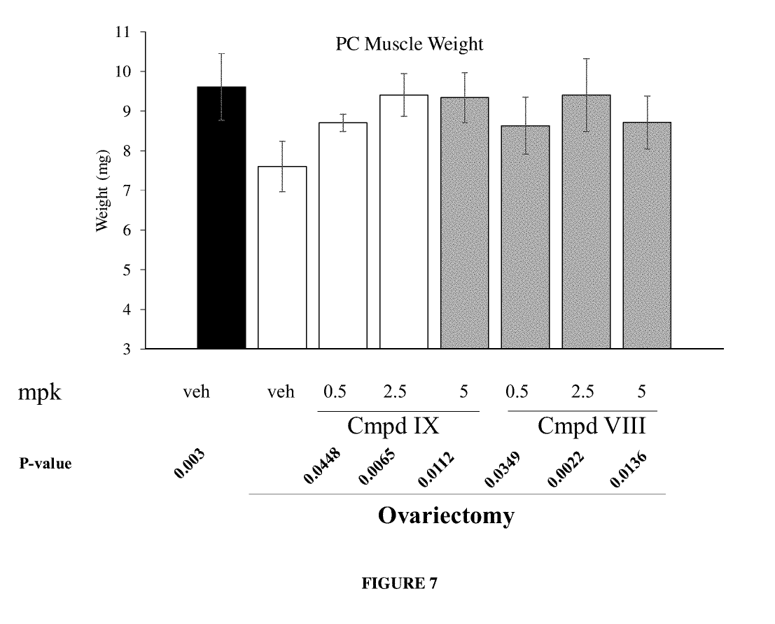

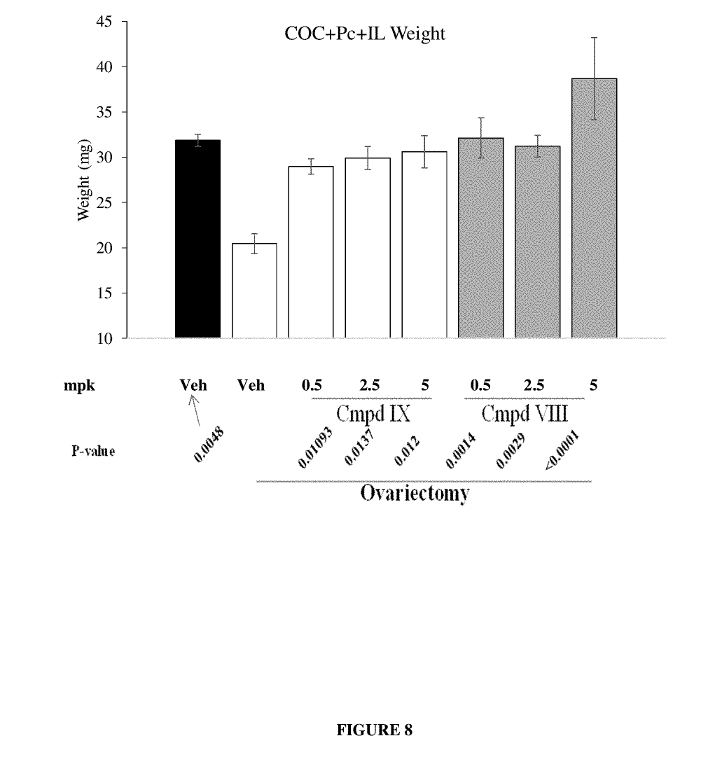

Of the three muscle types, the COC was more sensitive to ovariectomy (OVX). OVX reduced COC weight by approximately 50%, compared to intact animals (FIG. 6). SARMs dose-dependently increased the COC muscle attaining p values as low as 0.0001. The Pc was more modestly reduced by OVX (FIG. 7). Despite this, the SARMs increased Pc muscle weights significantly compared to OVX controls (p<0.05). The cumulative weight of the COC, Pc, and IL was also significantly increased by SARM treatment compared to OVX animals (p<0.001) (FIG. 8).

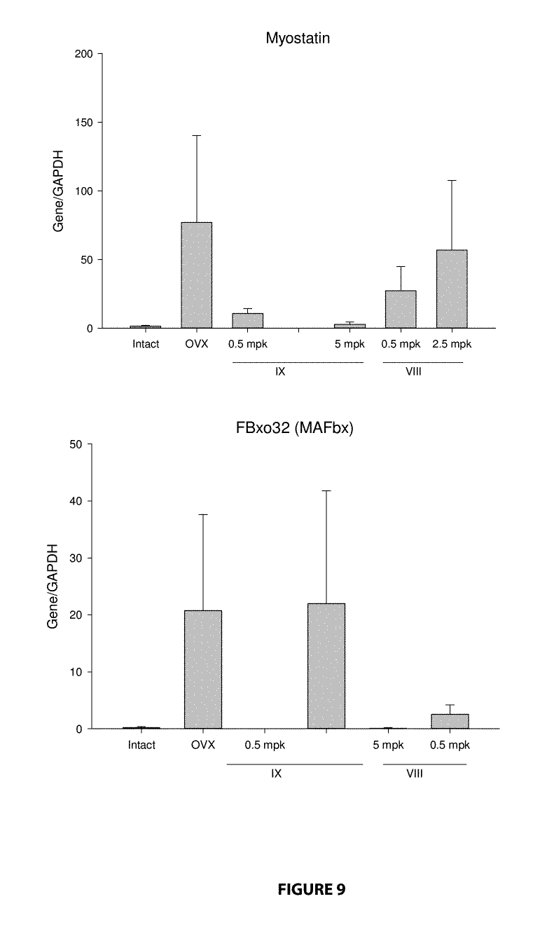

The expression of selected genes in the COC by real-time PCR demonstrated that OVX significantly increased the expression of two catabolic genes, myostatin and Fbxo32 or MAFbx (FIG. 9). Treatment with the SARMs reversed the increase in the expression of these genes and returned their expression to that of intact controls, an indication that SARMs block the muscle's catabolic pathways to increase muscle weight and strength.

Drug concentrations were measured in serum using LC-MS/MS and demonstrate a dose-dependent increase in the concentration of the SARMs (Table 1).

Animals were sacrificed 24 h after the last dose to measure the steady state concentration. Despite lower serum concentration, compound of Formula VIII performed better than compound of Formula IX in increasing the muscle weights.

TABLE 1
Serum concentrations of SARMs after 28 days of treatment.

| Drug | Dose (mpk) | Avg (nM) | S.E. (nM) |
|---|---|---|---|
| Vehicle | — | BLQ | BLQ |
| IX | 0.5 | 861.75 | 32.84 |
| IX | 2.5 | 3852 | 292.21 |
| IX | 5 | 6065 | 663.98 |
| VIII | 0.5 | 429 | 71.13 |
| VIII | 2.5 | 2436 | 210.13 |
| VIII | 5 | 4100 | 198.24 |

BLQ—below the limit of quantitation

### Conclusion:

This is the first study to clearly demonstrate that compounds of Formulas IX and VIII have the potential to increase the weight of pelvic floor muscles that were decreased by OVX. This increase in size of these critically important pelvic muscles has the potential to translate to the treatment of women with SUI.

From the mice data, it appears that COC is the principal muscle affected by estrogens. LA muscle (Pc+IL) are smaller than COC and are affected minimally by circulating estrogens. As both LA and COC muscles are important for maintenance of pelvic floor architecture, it is vital to compensate the loss of either one or both, whichever is affected. p values indicate that compound of Formula VIII might be a better drug than compound of Formula IX in increasing the pelvic floor muscle.

### Summary:

Objectives:

To evaluate the effect of non-steroidal SARMs on pelvic floor muscles in ovariectomized (OVX) female mice and to identify a dose that will strengthen the pelvic floor muscle without increasing the lean mass.

Methods:

Six to eight week old female mice (n=6-8/group) were ovariectomized (OVX) or sham operated. One month after OVX, when the serum hormone levels were at trough, body composition was measured by MRI and treatment was initiated with vehicle or a dose response of one of two SARMs. Twenty eight days after treatment initiation, body composition was again measured, animals were sacrificed, and pelvic floor muscles were weighed. Serum drug concentrations were measured by LC-MS/MS. Muscle sections were stained for collagen and elastin to evaluate the effect of SARMs on architecture. Data were analyzed by One Way ANOVA followed by Tukey test.

Results:

The doses of SARMs used in the study did not result in a significant increase in body weight or whole body lean mass. Ovariectomy significantly reduced the weight of coccygeous (COC) muscle by greater than 50%, illeococcygeous (IL) by 30% and the entire pelvic floor muscle mass by 50%, which were all reversed to the intact level by SARMs. The increase in pelvic floor muscle

directly correlated with the serum drug concentration. Catabolic genes such as myostatin and MuRF1 were inhibited by the SARMs. Histological studies indicate that the pelvic floor muscle fibers were hypertrophied in SARM-treated animals.

Conclusion:

SARMs have the potential to increase pelvic floor muscle mass and architecture and could be a potential treatment option for UI, including SUI.

### Example 3

### Compound of Formula IX as a Treatment for Stress Urinary Incontinence (SUI), Urge Urinary Incontinence (UUI), and Mixed Incontinence in Post-Menopausal Women

### A Proof of Concept Clinical Study

This was initially a single site, proof of concept feasibility study to describe the effect of the S-isomer of the compound of Formula IX (Compound IX) 3 mg in postmenopausal female subjects with SUI. However, data presented herein were from subjects seen at 3 separate sites.

Primary Objective:

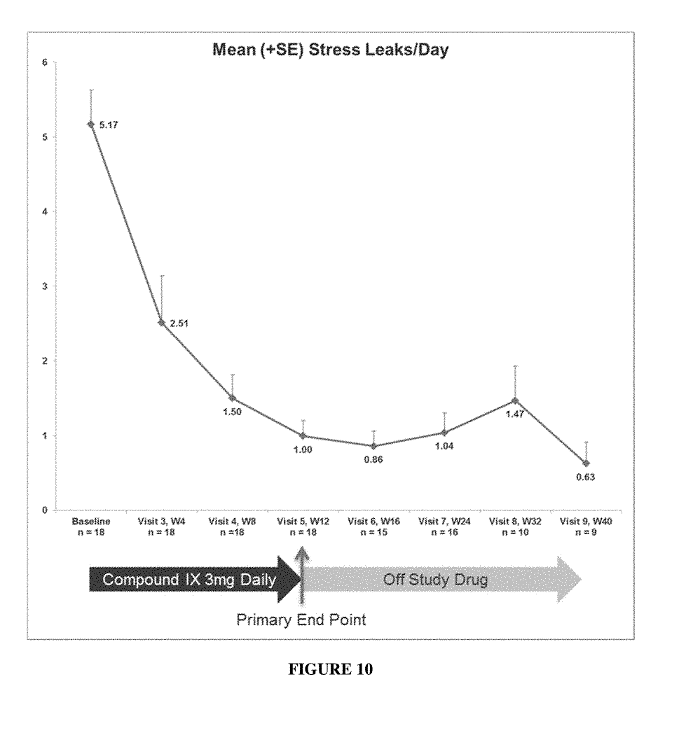

Described is the effect of 12 weeks of treatment of Compound IX on the number of stress urinary incontinence episodes/day as assessed by the 3 day voiding diary. See leaks/day data in Tables 2 (individual subject data from all 3 sites), Tables 4-6 (mean stress leaks for sites 1, 2, and 3, respectively), and FIG. 10 (mean stress leaks/day across all sites). Further, see Table 3 for the durability of this response in subjects assessed at 16, 24, 32 and 40 weeks (and their pad weights). The first 9 subjects to reach 40 weeks, i.e., 28 weeks after treatment with Compound IX was discontinued, demonstrated responses that were durable for at least 28 weeks after discontinuation.

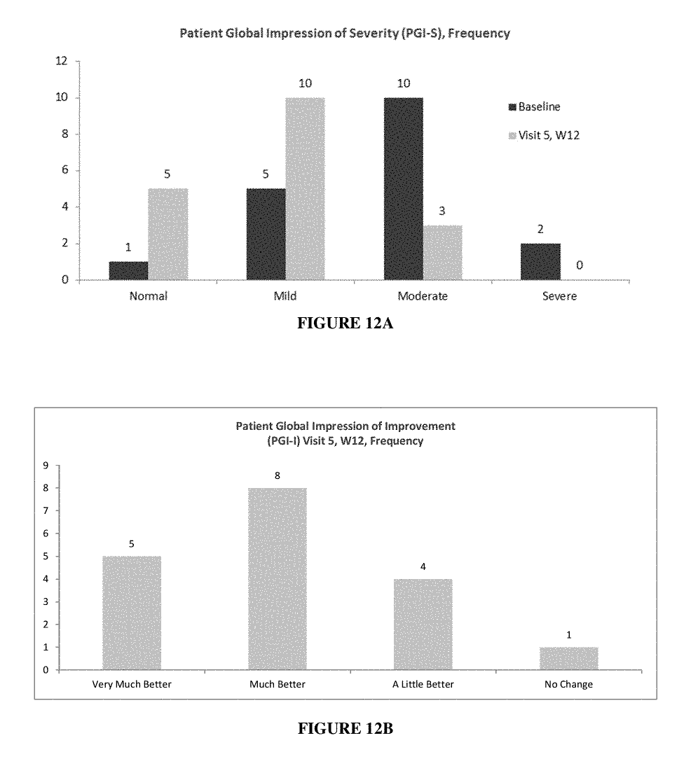

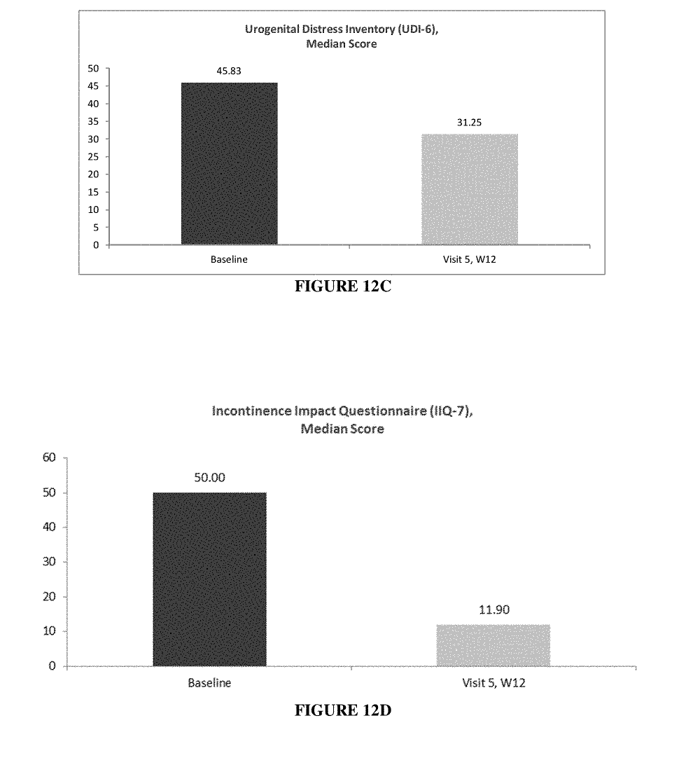

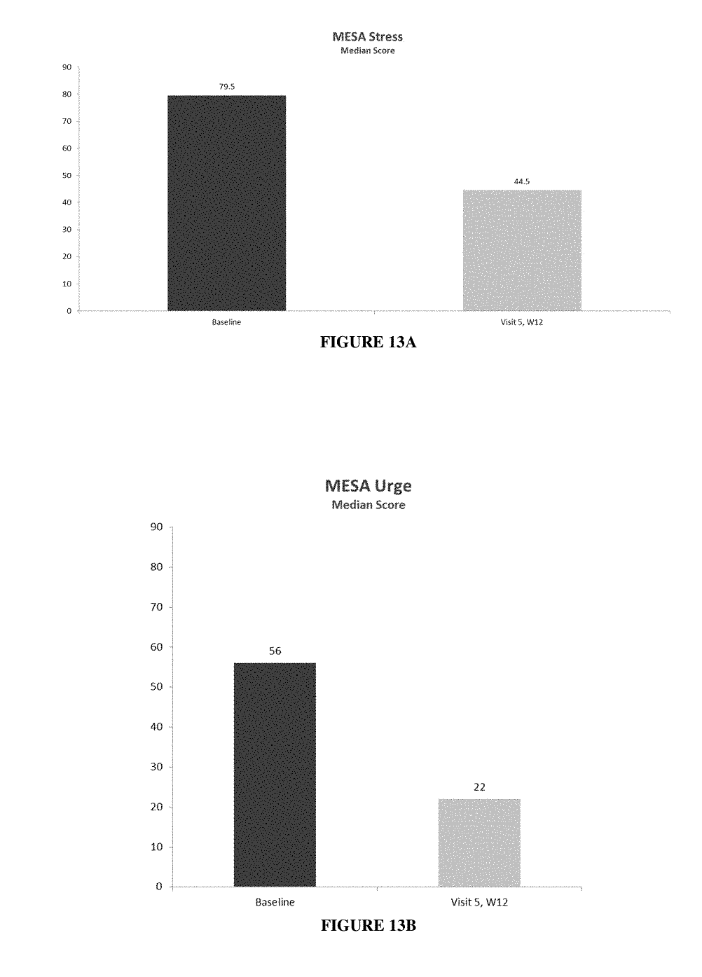

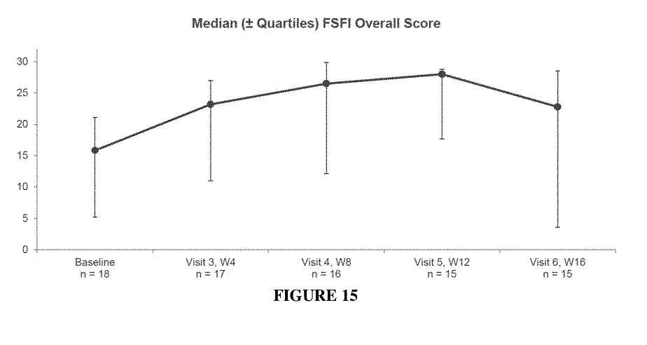

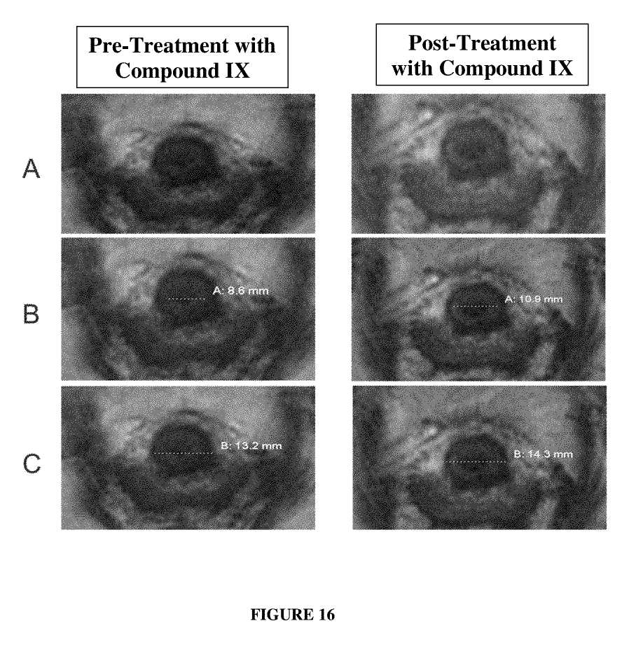

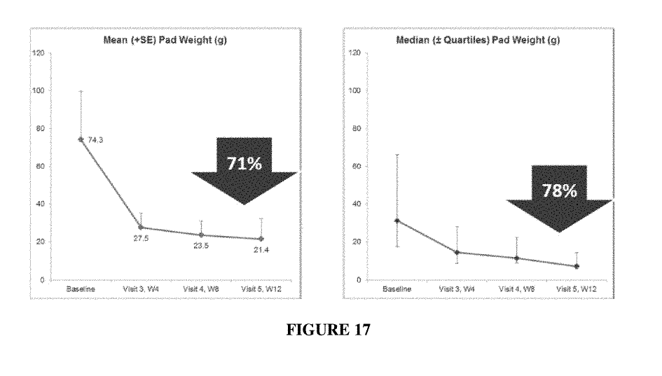

Secondary Objectives:
To describe the effect of 12 weeks of treatment of Compound IX on the number of voids/day as assessed by the 3 days voiding diary. (not shown herein)
To describe the effect of 12 weeks of treatment of Compound IX on urine volume per void as assessed by the 3 days voiding diary. (not shown herein)
Described is the effect of 12 weeks of treatment of Compound IX on SUI as assessed by 24 hours pad weight test. See Tables 2 (individual subject data) and Tables 4-6 (mean pad weight data for sites 1, 2, and 3, respectively), and FIG. 17 for mean and median reductions in pad weights from baseline to week 12 (across all sites).
To describe the effect of 12 weeks of treatment of Compound IX on SUI as assessed by the Urethral Pressure Profile (UPP). The UPP assessment was removed from the protocol because it is invasive and not standard of care. (not shown herein)
To describe the effect of 12 weeks of treatment of Compound IX on SUI as assessed by the Bladder Stress Test. (not shown herein)
Described is the effect of 12 weeks of treatment of Compound IX on subject reported stress urinary incontinence symptoms as assessed by the MESA Urinary Questionnaire. See summary data for all subjects across the 3 sites reported in Table 7 and FIGS. 13A and 13B.
Described is the effect of 12 weeks of treatment of Compound IX on subject reported impression of stress urinary incontinence severity as assessed by the Patient Global Impression of Severity Scale (PGI-S). See summary data for all subjects across the 3 sites reported in FIG. 12A.
Described is the effect of 12 weeks of treatment of Compound IX on subject reported impression of improvement as assessed by the Patient Global Impression of Improvement Scale (PGI-I). See summary data for all subjects across the 3 sites reported in FIG. 12B.
Described is the effect of 12 weeks of treatment of Compound IX on subject reported urogenital distress as assessed by the Urinary Distress Inventory Questionnaire (UDI-6). See summary data for all subjects across of 3 sites reported in Table 7 and FIG. 12C.
Described is the effect of 12 weeks of treatment of Compound IX on subject reported impact of urinary incontinence on daily life as assessed by the Incontinence Impact Questionnaire (IIQ-7). See summary data for all subjects across the 3 sites reported in Table 7 and FIG. 12D.
Described is the effect of 12 weeks of treatment of Compound IX on subject reported sexual function as indicated on the completion of the Female Sexual Function Index Questionnaire (FSFI). See summary data for all subjects across the 3 sites reported in Table 7 and FIG. 15.
Described is the effect of 12 weeks of treatment of Compound IX on pelvic floor muscles as measured by MRI. See FIG. 11 wherein the pelvic MRI for one representative subject on trial is reported at baseline and at 12 weeks, FIG. 16 wherein the pelvic MRI's for three representative subjects on trial are reported at baseline and at 12 weeks, and Example 19 and Table 13 which provide individual measurements of levator ani thickness, inner and outer diameter of the urethra, and endometrial thickness, and mean changes in the same from baseline to week 12.

Safety Objective:

Described is the safety profile of Compound IX 3 mg PO daily in subjects with SUI. See Table 8 which is historical data from other clinical trials establishing the safety profile of Compound IX in human subjects. See also the lack of increase in endometrial thickness in Example 19.

Target Population:

Adult postmenopausal women with SUI.

Study Duration:

12 weeks on study drug with durability assessment at 40 weeks, or 28 weeks after discontinuation of Compound IX.

Number of Subjects/Participation Duration:

At the time of writing, 19 subjects were enrolled in the study. Among the enrolled subjects, 18 subjects have completed the 12-week study and one subject did not continue from week 2. At the time of writing, only 9 subjects had completed the durability assessment at 40 weeks.

Indication for Product Use:

Compound IX has been previously tested as a treatment for muscle wasting associated with cancer cachexia among other clinical uses but is not currently marketed.

Instructions for Product Use:

Subjects were instructed to take one 3 mg softgel capsule per day by mouth, without regard to food intake.

Statistical Considerations:

This is a proof of concept feasibility study, so no power calculation was needed. Therefore, up to 35 subjects meeting inclusion/exclusion criteria would be recruited until the enrolled subjects have completed treatment. Further, durability was assessed at 40 weeks. Descriptive statistics were performed to explore changes in primary and secondary outcomes measures between baseline and weeks 12 (end of treatment) or 40 (durability). The primary efficacy measure was a reduction in the number of stress incontinence episodes/day. Secondary efficacy measures included reduction in number of voids per day, volume of voids (not reported yet), 24 hour pad weight, responses to validated questionnaires, changes in UPP measures (not reported yet), changes in sexual function, and changes in pelvic floor muscles as measured by MRI. Safety will be determined by the number and type of adverse events reported during treatment, but is reported here from historical data from other clinical trials. Various imputation methods may be explored.

Preliminary Studies Related to Stress Urinary Incontinence:

Extensive clinical data related to the use of Compound IX are described below; however, there are both preclinical and clinical data supporting the specific investigation of Compound IX for the treatment of SUI. Among the preclinical findings are that Compound IX has androgenic and anabolic activity in male and female rat models. Compound IX has consistently been observed to increase body weight, specifically muscle, in female rats. In a male rat model, with castrate levels of serum testosterone (similar to what might be expected in females), Compound IX has the ability to induce hypertrophy of the levator ani muscle to approximately 120% of an intact male. These studies together provide an approximation of the expected effect of Compound IX, since currently there are no data in female models regarding levator ani hypertrophy or stress urinary incontinence. Moreover, in two phase 3 studies (G300504 and G300505), 3 mg daily Compound IX resulted in a mild increase (approximately 1.7%) in lean body mass with no differential effect in male and female humans, however, no specific muscles were analyzed. Based upon these preclinical and clinical analyses, a significant growth/bulking of the levator ani in females with SUI was anticipated, which may also result in improvements in associated symptoms, and are therefore the focus of the study outlined herein.

### Study End Points:

Primary End Point:

Change in frequency of daily stress urinary incontinence episodes from Baseline to Week 12 and durability at 40 weeks (28 weeks after Compound IX treatment is discontinued).

Secondary End Points:

1. Change in frequency of daily voids from Baseline to Week 12.
2. Change in urine volume per void from Baseline to Week 12.
3. Change in 24 hours pad weight from Baseline to Week 12.
4. Change in maximum urethral closure pressure measurements from Baseline to Week 12.
5. Change in urine leakage (yes/no) on the Bladder Stress Test from Baseline to Week 12 as assessed while (a) coughing, and/or (b) performing a Valsalva maneuver.
6. Change in total score on the stress incontinence section of the MESA Urinary Questionnaire from Baseline to Week 12.
7. Change in Patient Global Impression of Severity (PGI-S) Scale from Baseline to Week 12.

### 8. Patient Global Impression of Improvement (PGI-I) Scale at Week 12.

9. Change in total score on the Urinary Distress Inventory (UDI-6) from Baseline to Week 12.
10. Change in total score on the Incontinence Impact Questionnaire (IIQ-7) from Baseline to Week 12.
11. Change in total score on the Female Sexual Function Index (FSFI) from Baseline to Week 12 as well as the change in subdomain scores: libido, arousal, lubrication, orgasm, satisfaction, and pain.
12. Change in pelvic floor muscles from Baseline to Week 12 as measured by MRI. Quantitative assessments may include the area of the levator hiatus, the anteroposterior and transverse diameters, and other relevant parameters.

Postmenopausal was defined as clinically confirmed female subjects who have undergone the onset of spontaneous, medical or surgical menopause prior to the start of this study.

### Results

At the time of writing, the results for this trial are almost finalized, and correspondingly, different numbers of subjects and slightly different values from previous reporting appear for different types of data. Data collection and/or analysis is still ongoing. However, the bulk of the data for the primary endpoint and many secondary endpoints have been collected and analyzed and are presented herein. The data presented herein are sufficient to establish the proof-of-concept that SUI in post-menopausal women can be treated with Compound IX.

### Positive Primary Endpoint Results

The data were collected from first 18 patients at 12 weeks and are derived from number of stress leaks reported by subjects in a 3 day voiding diary. The number of leaks/day at baseline and week 12 for individual subjects from all 3 sites are reported in Table 2 below. All subjects on trial demonstrated a response in the primary endpoint (50% or greater reduction in the number of stress leads/day; considered clinically meaningful) at week 12. Table 3 provides durability data of Compound IX for 18 patients and depicts that, for the first 9 subjects, this response was durable out to 40 weeks, i.e., 28 weeks (7 months) after treatment with Compound IX was discontinued. Data collection and analysis is ongoing. Further, the site of collection does not seem to bias the result as sites 1, 2, and 3, respectively, reported 86%, 75% and 78% reductions (Tables 4-6). The time course for mean stress leaks/day across all sites is shown in FIG. 10, which demonstrates a reduction of means stress leaks/day from 5.17 at baseline to 1.0 at week 12 (end of treatment) which is an 81% reduction, and this response appears durable at multiple time points out to 40 weeks (0.63 means stress leaks/day; N=9) or 7 months after discontinuation of study drug (FIG. 10). This is the first demonstration that post-menopausal women with SUI can be successfully treated with Compound IX, and serves as proof-of-concept for an ongoing, subsequent, larger and placebo controlled clinical trial. Unexpectedly, all patients were responders (50% or greater reduction in the number of stress leads/day; considered clinically meaningful) and the response was durable for at 7 months after discontinuation of Compound IX (Tables 2 and 3).

See Example 18 for a discussion of the unexpected effects of Compound IX on urge urinary incontinence (UUI) in patients on this proof-of-concept trial. See Example 19 for a discussion of the unexpected effects of Compound IX on the size of the levator ani muscle and urethral sphincter in subjects on this proof-of-concept trial.

FIG. 10 provides updated data for mean stress leaks which showed it was decreased by 81% by Compound IX. Adding in the new data, mean stress leaks decreased from 5.17 to 1.0 per day. All 18 patients were responders (50% or greater reduction in the number of stress leads/day; considered clinically meaningful). There was up to 7 months of durability in the reduction in stress incontinence episodes (N=9).

TABLE 2
Individual Efficacy Data: Stress Leaks Per Day and Pad Weights

| | | | | Pad Weights | Pad Weights | |
|---|---|---|---|---|---|---|
| Patient | Leaks/Day | Leaks/Day | % Reduction | at Baseline | at Week 12 | % Reduction |
| # | (Baseline) | (Week 12) | (Week 12) | (grams) | (grams) | (Week 12) |
| 1 | 10.33 | 1.33 | 87% | 28.93 | 37.27 | — |
| 2 | 4.33 | 0.67 | 85% | 33.04 | 6.48 | 80% |
| 3 | 6.33 | 0.33 | 95% | 9.18 | 6.71 | 27% |
| 4 | 8.33 | 2.33 | 72% | 60.47 | 7.31 | 88% |
| 5 | 3.33 | 0 | 100% | 17.25 | 7.74 | 55% |
| 6 | 3.33 | 1 | 70% | 9.39 | 4.54 | 52% |
| 7 | 4 | 1.33 | 67% | 48.85 | 26.93 | 45% |
| 8 | 4.33 | 0 | 100% | 198.12 | 3.58 | 98% |
| 9 | 3 | 0.33 | 89% | 8.9 | −2.3 | “126%” |
| 10 | 3.33 | 1.66 | 50% | 102.9 | 204.4 | — |
| 11 | 6 | 0.33 | 95% | 29.1 | 14.3 | 51% |
| 13 | 5.33 | 1 | 81% | 450 | 10.1 | 98% |
| 14 | 6 | 0 | 100% | 158.3 | 29 | 82% |
| 15 | 3 | 1.30 | 57% | 28 | 6.9 | 75% |
| 16 | 4.67 | .67 | 86% | 29 | 5.63 | 81% |
| 17 | 6 | 2.3 | 62% | 41.8 | 3.9 | 91% |
| 18 | 4.67 | .33 | 93% | 66.21 | 6.13 | 91% |
| 19 | 6.67 | 3 | 55% | 17.3 | 7.04 | 59% |

TABLE 3
Durability of Response (% reduction in leaks/day) at
12, 16, 24 32 and 40 Weeks*

| | | Durability of Response | | | |
|---|---|---|---|---|---|
| | Primary | Post-Treatment | | | |
| | Endpoint | % | % | % | |
| | % Reduction | Reduction | Reduction | Reduction | % Reduction |
| Patient | Week 12 | Week 16 | Week 24 | Week 32 | Week 40 |
| 1* | 87% | | | | |
| 2 | 85% | 77% | 69% | 54% | 69% |
| 3 | 95% | 100% | 100% | 95% | 100% |
| 4 | 72% | 100% | 100% | 100% | 100% |
| 5 | 100% | 100% | 100% | 100% | 100% |
| 6 | 70% | 60% | 70% | NA | 80% |
| 7 | 67% | 50% | 42% | 17% | 42% |
| 8 | 100% | 92% | 100% | 100% | 100% |
| 9 | 89% | 100% | | | |
| 10 | | | | | |
| 11 | 95% | 89% | 95% | 50% | 100% |
| 12 | 81% | 100% | 100% | | |
| 13 | 100% | NA | 100% | | |
| 14 | 57% | NA | 10% | | |
| 15 | 86% | 79% | 57% | 36% | 72% |
| 16 | 62% | 78% | 78% | 55% | |
| 17 | 93% | 64% | 50% | 93% | |
| 18 | 55% | 65% | 60% | | |

*Patient completed study prior to the durability amendment.
NA (not available) - diary not received.

Positive Secondary Endpoint Results

Reported herein are data for the first 18 patients at 12 weeks for the secondary endpoints of 24 hour pad weight test (Tables 2 and 4-6, and FIG. 17) and change in levator ani muscle, urethra, and endometrium for a selected subjects from Baseline to Week 12 as measured by quantitative MRI (FIGS. 11 and 16, Table 13 and Example 19). Data collection/analysis for these and additional secondary endpoints such as frequency of daily voids, urine volume per void, and bladder stress test is still ongoing.

Pad Weights:

The mean pad weights in grams (g) at baseline and week 12 for individual subjects from all 3 sites are reported in Table 2. By this measure, all subjects except patients 1 and 10 demonstrated a response at week 12. Further, the site of collection does not seems to bias the result as sites 1, 2, and 3, respectively, reported 77%, 66% and 74% reductions (Tables 4-6). Overall, the median pad weight decreased by 78%, from 31.07 grams at baseline to 6.97 grams at week 12; whereas mean pad weights decreased by 71% from 74.26 grams at baseline to 21.4 grams at week 12.

TABLE 4
Response rate observed at Site 1

| Site 1 (n = 10) | % Reduction |
|---|---|
| Stress Leaks | 86 |
| Mean Pad Weights (g) | 77 |

TABLE 5
Response rate observed at Site 2

| Site 2 (n = 5) | % Reduction |
|---|---|
| Stress Leaks | 75 |
| Mean Pad Weights (g) | 66 |

TABLE 6
Response rate oberverd at Site 3

| Site 3 (n = 2) | % Reduction |
|---|---|
| Stress Leaks | 78 |
| Mean Pad Weights (g) | 74 |

Levator Ani Width by MRI:

Quantitative MRI was used to visualize the pelvic floor of all post-menopausal subjects with SUI. Axial and coronal sections of a representative subject at baseline and 12 weeks (3 months) demonstrated that the levator ani muscle thickness increased by approximately 20% (FIG. 11). This represents the first data demonstrating the ability to increase levator ani size in human, and suggests that the Compound IX may be able to affect the architecture of the pelvic floor.

See Example 19 for further MRI data collected in this proof-of-concept clinical trial, including further levator ani thickness data, urethral sphincter inner and outer diameter data, and endometrial thickness data. MRI data, taken together with the functional data above, suggest that Compound IX is a promising agent for post-menopausal SUI or UUI (see also below and Example 18), or mixed incontinences (see also below and Example 18), without proliferative effects on the uterus. The latter effect suggests that the scope of Compound IX use in urinary incontinence could be expanded to include pre- or peri-menopausal women.

Improvements in Quality of Life (QOL) Across all Measures

The data were collected from 18 patients at 12 weeks. Data collection/analysis for additional secondary endpoints are ongoing. Women reported improved quality of life measurements in each of the five instruments collected in the study, including the Patient Global Impression of Improvement (PGI-I) and Female Sexual Function Index (FSFI). FIG. 15 provides the FSFI overall scores from baseline to 16 weeks.

At week 12, 17 of 18 patients showed improved PGI-I scores and median MESA Stress, MESA Urge, and FSFI scores improved from a baseline score of 79.5 to 44.5, 56 to 22, and 15.90 to 28.05, respectively, at week 12. Summary values of these and for other QOL metrics are reported in Table 7 below, each reflecting improvements at 12 weeks. FIGS. 12A (PGI-S), 12B (PGI-I), 12C (UDI-6), and 12D (IIQ-7) provide depictions of the improvements in quality of life (QOL) across all measures by Compound IX. The data were obtained from 18 patients at 12 weeks.

The QOL measurements are described in greater detail above and are well-known and validated measures.

TABLE 7
Improvements in QOL

| QOL Measure | Baseline | 12 Weeks |
|---|---|---|
| FSFI | 15.90 | 28.20 |
| UDI-6 | 45.83 | 31.25 |
| IIQ-7 | 50.00 | 11.90 |
| MESA Stress | 79.5 | 44.5 |
| MESA Urge | 56 | 22 |

FSFI: sexual functioning in women (scale range 2-36)
PGI-I: response of a condition to a therapy (scale range 1-7)
PGI-S: rates severity of a specific condition (scale range 1-4)
UDI-6: degree to which symptoms are troubling (scale range 0-100)
IIQ-7: impact of UI on activities/emotions in women (scale range 0-100)
MESA Stress: rates the medical, epidemiologic and social aspects of aging (MESA) score. Data from 18 patients at 12 weeks. These are the SUI questions.
MESA Urge: rates the medical, epidemiologic and social aspects of aging (MESA) score. Data from 18 patients at 12 weeks. These are the UUI questions.

For the PGI-S measure, 12 patients reported symptoms as “moderate” or “severe” at baseline; whereas only 3 patients reported “moderate” symptoms and no patients reported “severe” at 12 weeks (FIG. 12A). There were no serious adverse events reported and reported adverse events were minimal and included headaches, nausea, fatigue, hot flashes, insomnia, muscle weakness and acne. Mild transient elevations in liver enzymes were observed, as well as reductions in total cholesterol, LDL, HDL and triglycerides. Reductions in SHBG consistent with androgen biology were observed. No changes in endometrial stripe thickness were observed. These are all consistent with the previously compiled safety profile summarized in Table 8.

TABLE 8
Safety Profile: Compound of Formula IX

| | Compound IX | | Placebo | | All subjects | |
|---|---|---|---|---|---|---|
| MedDRA Preferred Term | (N = 896) | | (N = 437) | | (N = 1333) | |
| Any Treatment Related | 219 | (24.4) | 73 | (16.7) | 292 | (21.9) |
| Adverse Event | | | | | | |
| Headache | 51 | (5.7) | 10 | (2.3) | 61 | (4.6) |
| Nausea | 27 | (3.0) | 12 | (2.7) | 39 | (7.9) |
| Alanine | 19 | (2.1) | 2 | (0.5) | 21 | (1.6) |
| Aminotransferase | | | | | | |
| Increased | | | | | | |
| Diarrhea | 19 | (2.1) | 12 | (2.7) | 31 | (2.3) |
| Dizziness | 18 | (2.0) | 2 | (0.5) | 20 | (1.5) |
| Back pain | 13 | (1.5) | 2 | (0.5) | 15 | (1.1) |
| Constipation | 12 | (1.3) | 3 | (0.7) | 15 | (1.1) |
| Vomiting | 12 | (1.3) | 4 | (0.9) | 16 | (1.2) |
| Pain in Extremity | 11 | (1.2) | 4 | (0.9) | 15 | (1.1) |
| Hyperhidrosis | 9 | (1.0) | 1 | (0.2) | 10 | (0.8) |
| Pruritus | 9 | (1.0) | 3 | (0.7) | 12 | (0.9) |
| Somnolence | 9 | (1.0) | 0 | (0) | 9 | (0.7) |
| Dyspnea | 8 | (0.9) | 0 | (0) | 8 | (0.6) |
| Fatigue | 8 | (0.9) | 5 | (1.1) | 13 | (1.0) |
| Abdominal Pain | 7 | (0.8) | 2 | (0.5) | 9 | (0.7) |
| Hot Flush | 6 | (0.7) | 2 | (0.5) | 8 | (0.6) |
| Muscle Spasms | 6 | (0.7) | 1 | (0.2) | 7 | (0.5) |
| Myalgia | 6 | (0.7) | 1 | (0.2) | 7 | (0.5) |
| Dizziness Postural | 5 | (0.6) | 0 | (0) | 5 | (0.4) |
| Insomnia | 5 | (0.6) | 1 | (0.2) | 6 | (0.5) |
| Rash | 5 | (0.6) | 0 | (0) | 5 | (0.4) |

### Summary:

Consistent with previous findings, at the end of the pre-specified, 12-week treatment period, all of the 18 women treated with Compound IX showed clinically meaningful reductions in stress incontinence episodes per day (the primary endpoint of the trial).
Mean stress leaks decreased by 81 percent from baseline;
Stress leaks decreased from a mean of 5.17 leaks/day at baseline to 1.00 leak/day;
All 18 subjects demonstrated clinically meaningful reductions in stress incontinence episodes per day, compared to baseline, of at least 50 percent; and
Median Medical, Epidemiologic and Social Aspects of Aging (MESA) scores for SUI decreased from 79.5 percent to 44.5 percent.

The reduction in incontinence episodes was sustained, or durable, well beyond the 12-week treatment period.
Patients are being followed for up to seven months post-treatment to assess Compound IX's duration of effect. To date no patient, including all nine patients who have reached seven months, has returned to her baseline level of SUI episodes.

Women reported improved quality of life measurements in each of the four instruments collected in the study, including the Patient Global Impression of Improvement (PGI-I), Urogenital Distress Inventory (UDI-6) and Incontinence Impact Questionnaire (IIQ-7). Improvements in Female Sexual Function Index (FSFI) were also noted. At week 12:

17 of 18 patients showed improved PGI-I scores;

Median UDI-6 scores decreased from 45.83 at baseline to 31.25;

Median IIQ-7 scores decreased from 50.00 at baseline to 11.90; and

Median FSFI scores improved from a baseline score of 15.85 to 28.00.

These top-line clinical trial results demonstrated that a daily dose of Compound IX of 3 mg substantially improved stress urinary incontinence (SUI) in women, as well as related quality of life measurements. In this open-label clinical trial, all 18 subjects completing 12 weeks of treatment saw a clinically significant reduction (50 percent or greater) in stress leaks per day, compared to baseline, i.e., each achieved the primary endpoint of the trial. Mean stress leaks decreased by 81 percent from baseline over 12 weeks (5.17 leaks/day at baseline to 1.0 leaks/day at week 12), and the reductions in daily stress leaks following completion of treatment have been sustained as subjects are being followed for up to 7 months post-treatment to assess the durability of treatment effect (0.63 leaks/day at 40 weeks (7 months post-treatment) in the first nine subjects). No subject has relapsed to her baseline levels. Urge urinary incontinence was also observed in 11 subjects, and Compound IX produced a 68% reduction from baseline over 12 weeks in these patients, which was also observed to be durable for at least 7 months (N=7). Further, MRI demonstrated statistically significant effects to enlarge the levator ani muscle thickness, and urethral inner and outer diameters, providing a rationale for the unexpectedly robust clinical responses and durations of clinical responses observed. There were no serious adverse events reported and reported adverse events were minimal and included headaches, nausea, fatigue, hot flashes, insomnia, muscle weakness and acne. Mild transient elevations in liver enzymes were observed, as well as reductions in total cholesterol, LDL, HDL and triglycerides. All were consistent with AR pharmacology and previous clinical trials with Compound IX. Based on the results from this Phase 2 proof-of-concept study with Compound IX, a randomized, placebo-controlled Phase 2 clinical trial to evaluate the change in frequency of daily stress urinary incontinence episodes following 12 weeks of treatment was initiated. The trial will evaluate the safety and efficacy of Compound IX (1 mg and 3 mg) compared with placebo in postmenopausal women with SUI. Compound IX has previously been evaluated in clinical trials enrolling in excess of 1,700 patients, in which approximately 1,200 individuals received doses ranging from 0.1 mg to 100 mg, and has been observed to be generally safe and well tolerated.

### Example 4

### Synthesis of Compound of Formula VIII

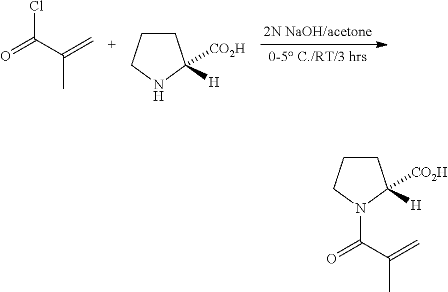

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, room temperature (RT)), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

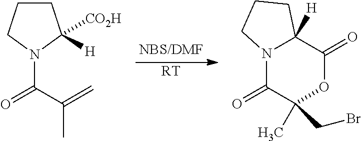

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

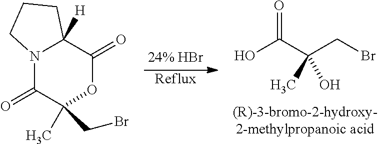

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.); ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm; [α]_{D}²⁶+10.5° (c=2.6, MeOH); Anal. Calcd. for C₄H₇BrO₃: C, 26.25, H, 3.86. Found: C, 26.28, H, 3.75.

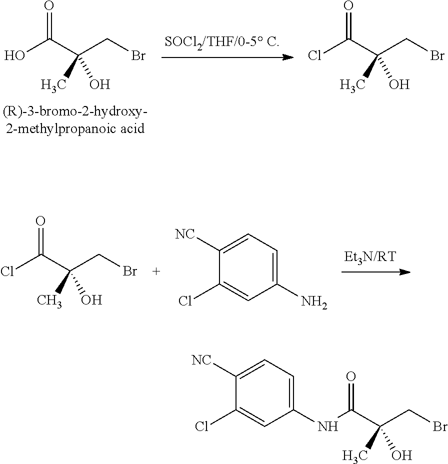

### Synthesis of (2R)-3-Bromo-N-(3-chloro-4-cyanophenyl)-2-hydroxy-2-methylpropanamide

Thionyl chloride (7.8 g, 65.5 mmol) was added dropwise to a cooled solution (less than 4° C.) of (R)-3-bromo-2-hydroxy-2-methylpropanoic acid (9.0 g, 49.2 mol) in 50 mL of THF under an argon atmosphere. The resulting mixture was stirred for 3 h under the same condition. To this was added Et₃N (6.6 g, 65.5 mol) and stirred for 20 min under the same condition. After 20 min, 4-amino-2-chlorobenzonitrile (5.0 g, 32.8 mmol) and 100 mL of THF were added and then the mixture was allowed to stir overnight at RT. The solvent was removed under reduced pressure to give a solid which was treated with 100 mL of H₂O, extracted with EtOAc (2×150 mL). The combined organic extracts were washed with saturated NaHCO₃ solution (2×100 mL) and brine (300 mL), successively. The organic layer was dried over MgSO₄ and concentrated under reduced pressure to give a solid which was purified from column chromatography using EtOAc/hexane (50:50) to give 7.7 g (49.4%) of target compound as a brown solid.

¹H NMR (CDCl₃/TMS) δ 1.7 (s, 3H, CH₃), 3.0 (s, 1H, OH), 3.7 (d, 1H, CH), 4.0 (d, 1H, CH), 7.5 (d, 1H, ArH), 7.7 (d, 1H, ArH), 8.0 (s, 1H, ArH), 8.8 (s, 1H, NH). MS: 342.1 (M+23). Mp 129° C.

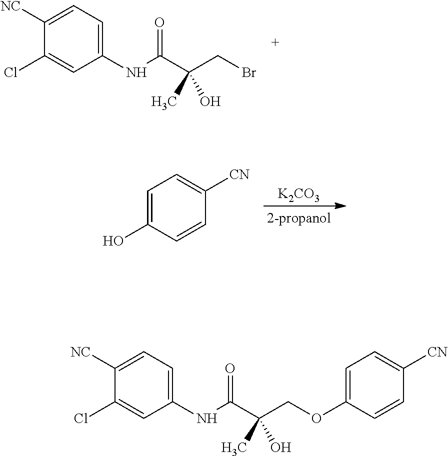

### Synthesis of (S)—N-(3-Chloro-4-cyanophenyl)-3-(4-cyanophenoxy)-2-hydroxy-2-methylpropanamide (Compound of Formula VIII)

A mixture of bromoamide (2.0 g, 6.3 mmol), anhydrous K₂CO₃ (2.6 g, 18.9 mmol) in 50 mL of acetone was heated to reflux for 2 h and then concentrated under reduced pressure to give a solid. The resulting solid was treated with 4-cyanophenol (1.1 g, 9.5 mmol) and anhydrous K₂CO₃ (1.7 g, 12.6 mmol) in 50 mL of 2-propanol was heated to reflux for 3 h and then concentrated under reduced pressure to give a solid. The residue was treated with 100 mL of H₂O and then extracted with EtOAc (2×100 mL). The combined EtOAc extracts were washed with 10% NaOH (4×100 mL) and brine, successively. The organic layer was dried over MgSO₄ and then concentrated under reduced pressure to give an oil which was purified by column chromatography using EtOAc/hexane (50:50) to give a solid. The solid was recrystallized from CH₂Cl₂/hexane to give 1.4 g (61.6%) of (S)—N-(3-chloro-4-cyanophenyl)-3-(4-cyanophenoxy)-2-hydroxy-2-methylpropanamide as a colorless solid.

¹H NMR (CDCl₃/TMS) δ 1.61 (s, 3H, CH₃), 3.25 (s, 1H, OH), 4.06 (d, J=9.15 Hz, 1H, CH), 4.50 (d, J=9.15 Hz, 1H, CH), 6.97-6.99 (m, 2H, ArH), 7.53-7.59 (m, 4H, ArH), 7.97 (d, J=2.01 Hz, 1H, ArH), 8.96 (s, 1H, NH). Calculated Mass: 355.1, [M+Na]⁺378.0. Mp: 103-105° C.

### Example 5

### Preclinical Anabolic and Androgenic Pharmacology of Compound of Formula VIII in Intact and Castrate Male Rats

Anabolic and androgenic efficacy of compound of Formula VIII administered by daily oral gavage were tested. The S-isomer of compound of Formula VIII was synthesized and tested as described herein.

### Materials and Methods:

Male Sprague-Dawley rats weighing approximately 200 g were purchased from Harlan Bioproducts for Science (Indianapolis, Ind.). The animals were maintained on a 12 h light/dark cycle with food (7012C LM-485 Mouse/Rat Sterilizable Diet, Harlan Teklad, Madison, Wis.) and water available ad libitum. The anabolic and androgenic activity of compound of Formula VIII in intact animals was tested, as well as a dose response evaluation in acutely orchidectomized (ORX) animals. Regenerative effects of the compound of Formula VIII in chronically (9 days) ORX rats was similarly evaluated.

The test article for this study was weighed and dissolved in 10% DMSO (Fisher) diluted with PEG 300 (Acros Organics, NJ) for preparation of the appropriate dosage concentrations. The animals were housed in groups of 2 to 3 animals per cage. Animals were randomly assigned to one of seven groups consisting of 4 to 5 animals per group. Control groups (intact and ORX) were administered vehicle daily. Compound of Formula VIII was administered via oral gavage at doses of 0.01, 0.03, 0.1, 0.3, 0.75, and 1 mg/day to both intact and ORX groups. Where appropriate, animals were castrated on day one of the study. Treatment with compound of Formula VIII began nine days post ORX and was administered daily via oral gavage for fourteen days.

The animals were sacrificed under anesthesia (ketamine/xyalzine, 87:13 mg/kg) and body weights were recorded. In addition, ventral prostate, seminal vesicles, and levator ani muscle were removed, individually weighed, normalized to body weight, and expressed as a percentage of intact control. Student's T-test was used to compare individual dose groups to the intact control group. Significance was defined a priori as a P-value <0.05. Ventral prostate and seminal vesicle weights were evaluated as a measure of androgenic activity, whereas levator ani muscle weight was evaluated as a measure of anabolic activity. Blood was collected from the abdominal aorta, centrifuged, and sera were frozen at −80° C. prior to determination of serum hormone levels. Serum luteinizing hormone (LH) and follicle stimulating hormone (FSH) concentrations were determined.

### Results:

A series of dose-response studies in intact and castrated rats in order to evaluate the potency and efficacy of compound of Formula VIII in both androgenic (prostate and seminal vesicles) and anabolic (levator ani muscle) tissue was conducted. In intact animals, compound of Formula VIII treatment resulted in decreases in the weight of both prostate and seminal vesicles while the levator ani muscle weight was significantly increased. Levator ani muscle weight following compound of Formula VIII treatment were 107%±5%, 103%±7%, 97%±7%, 103%±5%, 118%±7%, and 118%±7% of intact controls following doses of 0.01, 0.03, 0.1, 0.3, 0.75, and 1 mg/day, respectively. The prostate weights were 103%±10%, 99%±10%, 58%±10%, 58%±15%, 65%±20%, and 77%±23% of intact controls following doses of 0.01, 0.03, 0.1, 0.3, 0.75, and 1 mg/day, respectively. These results are significant since current androgen therapies are contraindicated in some patient populations due to the proliferative androgenic effects in prostate and breast tissues. However, many patients in these populations could benefit from the anabolic actions of androgens in muscle and bone. Since compound of Formula VIII exhibited tissue selective anabolic effects, it may be possible to treat patient groups in which androgens were contraindicated in the past.

In castrated, ORX animals, prostate weights following compound of Formula VIII treatment were 12%±2%, 17%±6%, 31%±3%, 43%±15%, 54%±17%, 58%±10%, and 73%±12% of intact controls following doses of 0, 0.01, 0.03, 0.1, 0.3, 0.75, and 1 mg/day, respectively. Similarly, seminal vesicle weights were 10%±2%, 10%±3%, 13%±4%, 21%±6%, 43%±8%, 51%±9%, and 69%±14% of intact controls following doses of 0, 0.01, 0.03, 0.1, 0.3, 0.75, and 1 mg/day, respectively. Significant increases were seen in levator ani muscle weights of in all dose groups, when compared to intact controls. The levator ani muscle weights were 40%±5%, 52%±8%, 67%±9%, 98%±10%, 103%±12%, 105%±12% and 110%±17% of intact controls corresponding to 0, 0.01, 0.03, 0.1, 0.3, 0.75, and 1.0 mg/day dose groups, respectively.

Testosterone propionate (TP) and S-3-(4-acetylaminophenoxy)-2-hydroxy-2-methyl-N-(4-nitro-3-trifluoromethylphenyl) propionamide (compound of Formula XII), maximally stimulated the levator ani muscle weight to 104% and 101%, respectively. These data show that compound of Formula VIII exhibited significantly greater efficacy and potency than either TP or compound of Formula XII. As a whole, these data show that compound of Formula VIII is able to stimulate muscle growth in the presence or absence of testosterone while exerting anti-proliferative effects on the prostate. These data show that the compound of Formula VIII restores lost muscle mass in patients with sarcopenia or cachexia. Additionally, the antiproliferative effects of the compound of Formula VIII on the prostate may allow some patient populations, in which androgens are currently contraindicated, access to anabolic agents.

Anabolic ratios were derived comparing muscle/prostate weight in castrated rats. Values obtained were 3.02, 2.13, 2.27, 1.90, 1.83 and 1.51 following doses of 0.01, 0.03, 0.1, 0.3, 0.75 and 1 mg/day, respectively.

Animals receiving 1 mg/day of compound of Formula VIII exhibited a prostate weight of 77%±23% and levator ani muscle weight of 118%±7% of intact control values, respectively. Compound of Formula VIII maintained prostate weight following orchidectomy at 73±12% of intact controls and levator ani muscle weight at 110±17% of intact controls. A derived dose of 0.1 mg/day of compound of Formula VIII would restore levator ani muscle weight to 100%, while such dose would only restore 43±15% prostate weight.

### Example 6

### Synthesis of Compound of Formula IX

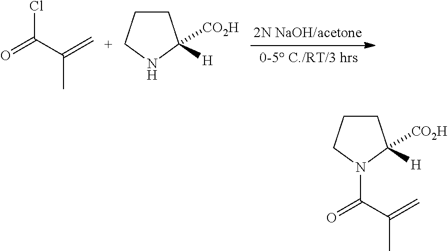

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° during the addition of the methacryloyl chloride. After stirring (3 h, RT), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.); the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

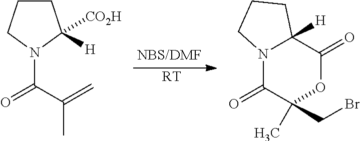

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

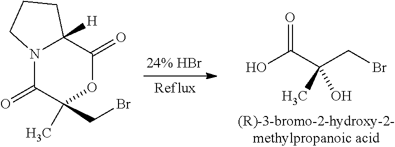

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.; ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd. for C₄H₇BrO₃: C, 26.25, H, 3.86. Found: C, 26.28, H, 3.75.

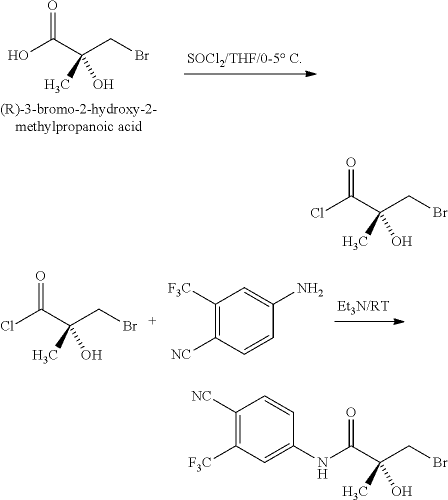

### Synthesis of (2R)-3-Bromo-N-[4-cyano-3-(trifluoromethyl)phenyl]-2-hydroxy-2-methylpropanamide

Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at RT. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M-H]⁻ 349.0. M.p.: 124-126° C.

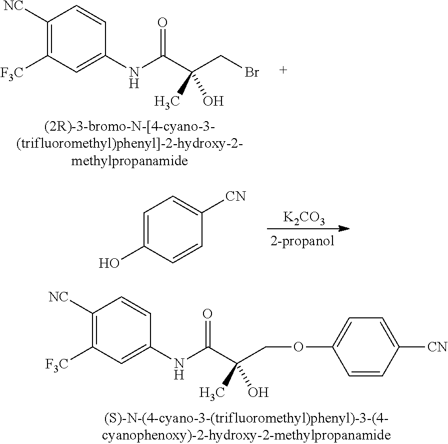

### Synthesis of (S)—N-(4-Cyano-3-(trifluoromethyl)phenyl)-3-(4-cyanophenoxy)-2-hydroxy-2-methylpropanamide (Compound of Formula IX)

A mixture of bromoamide ((2R)-3-bromo-N-[4-cyano-3-(trifluoromethyl)phenyl]-2-hydroxy-2-methylpropanamide, 50 g, 0.14 mol), anhydrous K₂CO₃ (59.04 g, 0.43 mol), 4-cyanophenol (25.44 g, 0.21 mol) in 500 mL of 2-propanol was heated to reflux for 3 h and then concentrated under reduced pressure to give a solid. The resulting residue was treated with 500 mL of H₂O and then extracted with EtOAc (2×300 mL). The combined EtOAc extracts were washed with 10% NaOH (4×200 mL) and brine. The organic layer was dried over MgSO₄ and then concentrated under reduced pressure to give an oil which was treated with 300 mL of ethanol and an activated carbon. The reaction mixture was heated to reflux for 1 h and then the hot mixture was filtered through Celite®. The filtrate was concentrated under reduced pressure to give an oil. This oil was purified by column chromatography using CH₂Cl₂/EtOAc (80:20) to give an oil which was crystallized from CH₂Cl₂/hexane to give 33.2 g (59.9%) of (S)—N-(4-cyano-3-(trifluoromethyl)phenyl)-3-(4-cyanophenoxy)-2-hydroxy-2-methylpropanamide as a colorless solid (a cotton type).

¹H NMR (CDCl₃/TMS) δ 1.63 (s, 3H, CH₃), 3.35 (s, 1H, OH), 4.07 (d, J=9.04 Hz, 1H, CH), 4.51 (d, J=9.04 Hz, 1H, CH), 6.97-6.99 (m, 2H, ArH), 7.57-7.60 (m, 2H, ArH), 7.81 (d, J=8.55 Hz, 1H, ArH), 7.97 (dd, J=1.95, 8.55 Hz, 1H, ArH), 8.12 (d, J=1.95 Hz, 1H, ArH), 9.13 (bs, 1H, NH). Calculated Mass: 389.10, [M-H]⁻ 388.1. Mp: 92-94° C.

### Example 7

### Androgenic & Anabolic Activity in Intact and ORX Rats of Compound of Formula IX

### Materials and Methods

Male Sprague-Dawley rats weighing approximately 200 g were purchased from Harlan Bioproducts for Science (Indianapolis, Ind.). The animals were maintained on a 12 h light/dark cycle with food (7012C LM-485 Mouse/Rat Sterilizable Diet, Harlan Teklad, Madison, Wis.) and water available ad libitum. Anabolic and androgenic activity of compound of Formula IX in intact animals was evaluated, and the dose response in acutely orchidectomized (ORX) animals was evaluated as well. Regenerative effects of compound of Formula IX in chronically (9 days) ORX rats were also assessed.

The compound was weighed and dissolved in 10% DMSO (Fisher) diluted with PEG 300 (Acros Organics, NJ) for preparation of the appropriate dosage concentrations. The animals were housed in groups of 2 to 3 animals per cage. Intact and ORX animals were randomly assigned to one of seven groups consisting of 4 to 5 animals per group. Control groups (intact and ORX) were administered vehicle daily. Compound of Formula IX was administered via oral gavage at doses of 0.01, 0.03, 0.1, 0.3, 0.75, and 1 mg/day to both intact and ORX groups.

Castrated animals (on day one of the study) were randomly assigned to dose groups (4-5 animals/group) of 0.01, 0.03, 0.1, 0.3, 0.75, and 1 mg/day, for dose-response evaluation. Dosing began nine days post ORX and was administered daily via oral gavage for fourteen days. The animals were sacrificed under anesthesia (ketamine/xyalzine, 87:13 mg/kg) after a 14-day dosing regimen, and body weights were recorded. In addition, ventral prostate, seminal vesicles, and levator ani muscle were removed, individually weighed, normalized to body weight, and expressed as a percentage of intact control. Student's T-test was used to compare individual dose groups to the intact control group. Significance was defined a priori as a P-value <0.05. As a measure of androgenic activity, ventral prostate and seminal vesicle weights were evaluated, whereas levator ani muscle weight was evaluated as a measure of anabolic activity. Blood was collected from the abdominal aorta, centrifuged, and sera were frozen at −80° C. prior to determination of serum hormone levels. Serum lutenizing hormone (LH) and follicle stimulating hormone (FSH) concentrations were determined.

### Results:

In intact animals, prostate weights following compound of Formula IX treatment were 111%±21%, 88%±15%, 77%±17%, 71%±16%, 71%±10%, and 87%±13% of intact controls following doses of 0.01, 0.03, 0.1, 0.3, 0.75, and 1 mg/day, respectively. Similarly, seminal vesicle weights decreased to 94%±9%, 77%±11%, 80%±9%, 73%±12%, 77%±10%, and 88%±14% of intact controls following doses of 0.01, 0.03, 0.1, 0.3, 0.75, and 1 mg/day, respectively. Significant increases were seen in levator ani muscle weights of sham animals, however, in all dose groups, when compared to intact controls. The levator ani muscle weights were 120%±12%, 116%±7%, 128%±7%, 134%±7%, 125%±9%, and 146%±17% of intact controls corresponding to 0.01, 0.03, 0.1, 0.3, 0.75, and 1.0 mg/day dose groups, respectively.

Compound of Formula IX partially maintained prostate weight following orchidectomy. Prostate weight in vehicle treated ORX controls decreased to 5%±1% of intact controls. At doses of 0.01, 0.03, 0.1, 0.3, 0.75, and 1.0 mg/day, compound of Formula IX maintained prostate weights at 8%±2%, 20%±5%, 51%±19%, 56%±9%, 80%±28%, and 74±12.5% of intact controls, respectively. In castrated controls, seminal vesicle weight decreased to 13%±2% of intact controls. Compound of Formula IX partially maintained seminal vesicle weights in ORX animals. Seminal vesicle weights from drug treated animals were 12%±4%, 17%±5%, 35%±10%, 61%±15%, 70%±14%, and 80%±6% of intact controls, following doses of 0.01, 0.03, 0.1, 0.3, 0.75, and 1.0 mg/day, respectively. In ORX controls the levator ani muscle weight decreased to 55%±7% of intact controls. We observed an anabolic effect in the levator ani muscle of compound of Formula IX treated animals. Compound of Formula IX fully maintained levator ani muscle weights at doses >0.1 mg/day. Doses >0.1 mg/day resulted in significant increases in levator ani weight compared to that observed in intact controls. Levator ani muscle weights as a percentage of intact controls were 59%±6%, 85%±9%, 112%±10%, 122%±16%, 127±12%, and 129.66±2% for the 0.01, 0.03, 0.1, 0.3, 0.75, and 1.0 mg/day dose groups, respectively. Eₘₐₓ and ED₅₀ values were determined in each tissue by nonlinear regression analysis in WinNonlin®. Eₘₐₓ values were 83%±25%, 85%±11%, and 131%±2% for prostate, seminal vesicles, and levator ani, respectively. The ED₅₀ in prostate, seminal vesicles, and levator ani was 0.09±0.07, 0.17±0.05, and 0.02±0.01 mg/day, respectively.

### Example 8

### Synthesis of Compound of Formula X

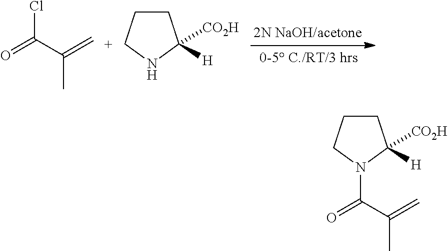

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, RT), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

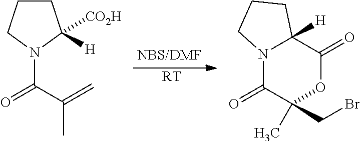

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

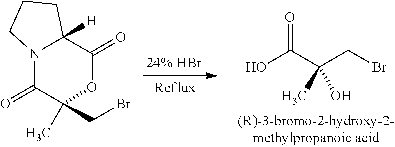

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.; ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd. for C₄H₇BrO₃: C, 26.25, H, 3.86. Found: C, 26.28, H, 3.75.

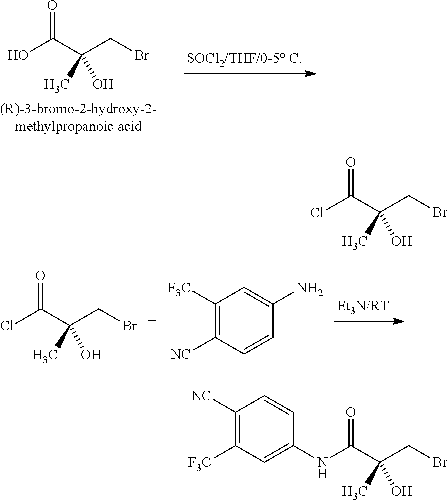

### Synthesis of (2R)-3-Bromo-N-[4-cyano-3-(trifluoromethyl)phenyl]-2-hydroxy-2-methylpropanamide

Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at RT. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M-H]⁻ 349.0. M.p.: 124-126° C.

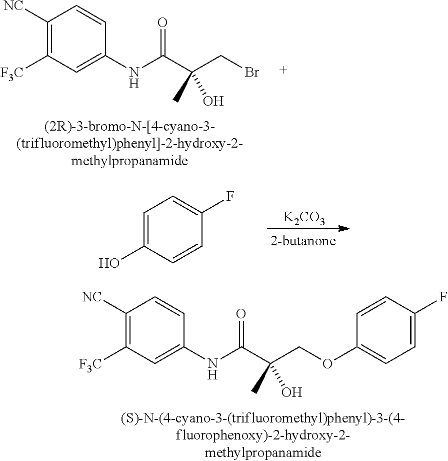

### Synthesis of (S)—N-(4-Cyano-3-(trifluoromethyl)phenyl)-3-(4-fluorophenoxy)-2-hydroxy-2-methylpropanamide (Compound of Formula X)

A mixture of bromoamide ((2R)-3-bromo-N-[4-cyano-3-(trifluoromethyl)phenyl]-2-hydroxy-2-methylpropanamide, 50 g, 0.14 mol), anhydrous K₂CO₃ (59.04 g, 0.43 mol), 4-fluorophenol (18.83 g, 0.17 mol) in 500 mL of 2-butanone was heated to reflux for 3 h and then concentrated under reduced pressure to give a solid. The resulting residue was treated with 500 mL of H₂O and then extracted with EtOAc (2×300 mL). The combined EtOAc extracts were washed with 10% NaOH (4×200 mL) and brine. The organic layer was dried over MgSO₄ and then concentrated under reduced pressure to give an oil which was treated with 300 mL of ethanol and an activated carbon. The reaction mixture was heated to reflux for 1 h and then the hot mixture was filtered through Celite®. The filtrate was concentrated under reduced pressure to give an oil. This oil was purified by column chromatography using CH₂Cl₂/EtOAc (80:20) to give an oil which was crystallized from CH₂Cl₂/hexane to give 40.2 g (75.2%) of (S)—N-(4-cyano-3-(trifluoromethyl)phenyl)-3-(4-fluorophenoxy)-2-hydroxy-2-methylpropanamide as a colorless solid.

¹H NMR (CDCl₃/TMS) δ 1.60 (s, 3H, CH₃), 3.41 (s, 1H, OH), 3.96 (d, J=9.0 Hz, CH), 4.45 (d, J=9.0 Hz, CH), 6.85-6.90 (m, 2H, ArH), 6.97-7.03 (m, 2H, ArH), 7.82 (d, J=8.4 Hz, 1H, ArH), 7.98 (dd, J=8.4, 2.1 Hz, 1H, ArH), 8.11 (d, J=2.1 Hz, ArH), 9.14 (bs, 1H, NH); Calculated Mass: 382.1 [M-H]⁻; Mp 143-144° C.

### Example 9

### Synthesis of Compound of Formula XI

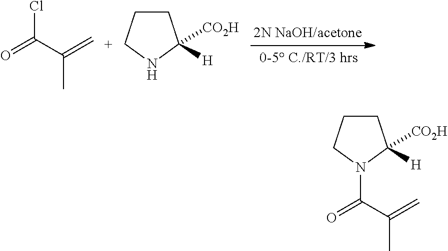

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, RT), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

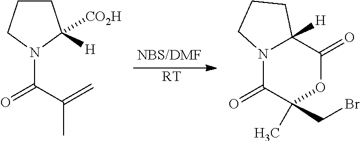

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

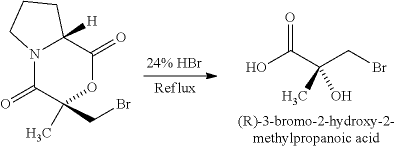

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.; ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd. for C₄H₇BrO₃: C, 26.25, H, 3.86. Found: C, 26.28, H, 3.75.

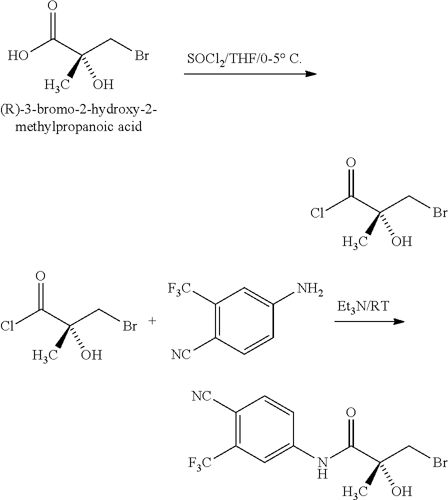

### Synthesis of (2R)-3-Bromo-N-[4-cyano-3-(trifluoromethyl)phenyl]-2-hydroxy-2-methylpropanamide

Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at RT. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M-H]⁻ 349.0. M.p.: 124-126° C.

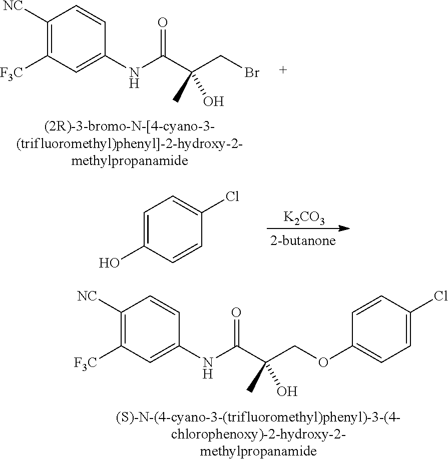

### Synthesis of (S)—N-(4-Cyano-3-(trifluoromethyl)phenyl)-3-(4-chlorophenoxy)-2-hydroxy-2-methylpropanamide (Compound of Formula XI)

A mixture of bromoamide ((2R)-3-bromo-N-[4-cyano-3-(trifluoromethyl)phenyl]-2-hydroxy-2-methylpropanamide, 50 g, 0.14 mol), anhydrous K₂CO₃ (59.04 g, 0.43 mol), 4-chlorophenol (21.60 g, 0.17 mol) in 500 mL of 2-butanone was heated to reflux for 3 h and then concentrated under reduced pressure to give a solid. The resulting residue was treated with 500 mL of H₂O and then extracted with EtOAc (2×300 mL). The combined EtOAc extracts were washed with 10% NaOH (4×200 mL) and brine. The organic layer was dried over MgSO₄ and then concentrated under reduced pressure to give an oil which was treated with 300 mL of ethanol and an activated carbon. The reaction mixture was heated to reflux for 1 h and then the hot mixture was filtered through Celite®. The filtrate was concentrated under reduced pressure to give an oil. This oil was purified by column chromatography using CH₂Cl₂/EtOAc (80:20) to give an oil which was crystallized from CH₂Cl₂/hexane to give 40.86 g (73.2%) of (S)—N-(4-cyano-3-(trifluoromethyl)phenyl)-3-(4-chlorophenoxy)-2-hydroxy-2-methylpropanamide as a colorless solid (a cotton type).

¹H NMR (CDCl₃/TMS) δ 1.63 (s, 3H, CH₃), 3.35 (s, 1H, OH), 4.07 (d, J=9.04 Hz, 1H, CH), 4.51 (d, J=9.0 Hz, 1H, CH), 6.97-6.99 (m, 2H, ArH), 7.57-7.60 (m, 2H, ArH), 7.81 (d, J=8.5 Hz, 1H, ArH), 7.97 (dd, J=2.0, 8.5 Hz, 1H, ArH), 8.12 (d, J=2.0 Hz, 1H, ArH), 9.13 (bs, 1H, NH); Calculated Mass: 398.1 [M-H]⁻.

### Example 10

### Androgenic & Anabolic Activity in Intact and ORX Rats of Compounds of Formulas X and XI

Animals.

Immature male Sprague-Dawley rats, weighing 90 to 100 g, were purchased from Harlan Biosciences (Indianapolis, Ind.). The animals were maintained on a 12 hour light-dark cycle with food and water available ad libitum.

Study Design.

Rats were randomly distributed into treatment groups groups. One day prior to the start of drug treatment, animals were individually removed from the cage, weighed and anesthetized with an intraperitoneal dose of ketamine/xylazine (87/13 mg/kg; approximately 1 mL per kg). When appropriately anesthetized (i.e., no response to toe pinch), the animals' ears were marked for identification purposes. Animals were then placed on a sterile pad and their abdomen and scrotum washed with betadine and 70% alcohol. The testes were removed via a midline scrotal incision, with sterile suture being used to ligate supra-testicular tissue prior to surgical removal of each testis. The surgical wound site was closed with sterile stainless steel wound clips, and the site cleaned with betadine. The animals were allowed to recover on a sterile pad (until able to stand) and then returned to their cage.

Twenty-four hours later, animals were re-anesthetized with ketamine/xylazine, and an Alzet osmotic pump(s) (model 2002) was placed subcutaneouly in the scapular region. In this instance, the scapular region was shaved and cleaned (betadine and alcohol) and a small incision (1 cm) made using a sterile scalpel. The osmotic pump was inserted and the wound closed with a sterile stainless steel wound clip. Animals were allowed to recover and were returned to their cage. Osmotic pumps contained the appropriate treatment dissolved in polyethylene glycol 300 (PEG300). Osmotic pumps were filled with the appropriate solution one day prior to implantation. Animals were monitored daily for signs of acute toxicity to drug treatment (e.g., lethargy, rough coat).

After 14 days of drug treatment, rats were anesthetized with ketamine/xylazine. Animals were then sacrificed by exsanguinations under anesthesia. A blood sample was collected by venipuncture of the abdominal aorta, and submitted for complete blood cell analysis. A portion of the blood was placed in a separate tube, centrifuged at 12,000 g for 1 minute, and the plasma layer removed and frozen at −20° C. The ventral prostates, seminal vesicles, levator ani muscle, liver, kidneys, spleen, lungs, and heart were removed, cleared of extraneous tissue, weighed, and placed in vials containing 10% neutral buffered formalin. Preserved tissues were sent for histopathological analysis.

For data analysis, the weights of all organs were normalized to body weight, and analyzed for any statistical significant difference by single-factor ANOVA. The weights of prostate and seminal vesicle were used as indexes for evaluation of androgenic activity, and the levator ani muscle weight was used to evaluate the anabolic activity.

The binding affinity of compound of Formula X is 3.3±0.08 nM. The binding affinity of compound of Formula XI is 3.4±0.08 nM.

The androgenic and anabolic activities of compound of Formula X were examined in a castrated rat model after 14 days of administration.

Compound of Formula X demonstrated tissue-selective pharmacological effects in castrated male rats, with higher efficacy in anabolic tissues (i.e. levator ani) as compared to androgenic tissues (i.e. prostate and seminal vesicles) (Table 9). Compound of Formula X demonstrated little pharmacologic activity in the prostate (8.7±1.39% of intact at 1.0 mg/day dose) and seminal vesicles (10.7±0.91% of intact at 1.0 mg/day dose), suggesting that it acts as a weak partial agonist in these tissues. Importantly, compound of Formula X demonstrates highly efficacious anabolic activity at 1.0 mg/day dose, returning the levator ani muscle to 75.2±9.51% of that observed in intact animals.

TABLE 9
Average (Mean ± S.D.) Organ Weights for
Compound of Formula X

| | Prostate | Levator Ani | Seminal Vesicles |
|---|---|---|---|
| Intact Control | 100 ± 11.28 | 100 ± 12.12 | 100 ± 2.48 |
| Castrated Control | 7.6 ± 0.68 | 45.9 ± 10.84 | 8.4 ± 1.05 |
| 0.10 mg/day | 6.4 ± 0.82 | 54.9 ± 5.77 | 8.8 ± 1.18 |
| 0.25 mg/day | 5.7 ± 0.61 | 61.0 ± 5.23 | 7.6 ± 1.37 |
| 0.50 mg/day | 6.2 ± 0.56 | 55.0 ± 9.23 | 9.3 ± 1.57 |
| 0.75 mg/day | 7.6 ± 0.74 | 68.9 ± 8.46 | 9.8 ± 3.65 |
| 1.00 mg/day | 8.7 ± 1.39 | 75.2 ± 9.51 | 10.7 ± 0.91 |

The androgenic and anabolic activities of the compound of Formula XI was examined in a castrated rat model after 14 days of administration.

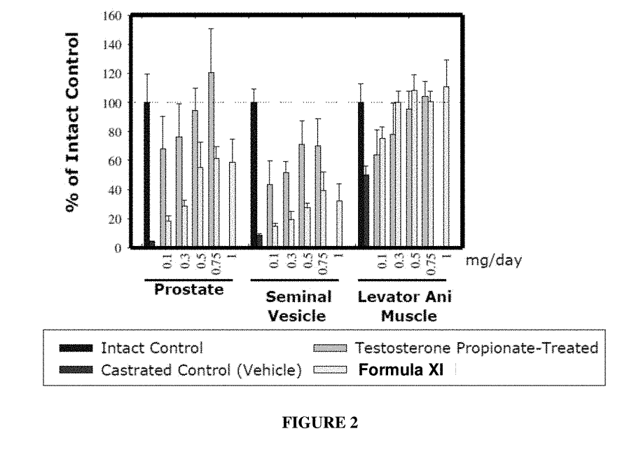

As shown in FIG. 2, the weights of prostate, seminal vesicle, and levator ani muscle in castrated, vehicle-treated rats decreased significantly, due to the ablation of endogenous androgen production. Exogenous administration of testosterone propionate, an androgenic and anabolic steroid, increased the weights of prostate, seminal vesicle, and levator ani muscle in castrated rats in a dose-dependent manner. Treatment with compound of Formula XI resulted in dose-dependent increases in prostate, seminal vesicle and levator ani muscle weights. Compared with testosterone propionate, compound of Formula XI showed lower potency and intrinsic activity in increasing the weights of prostate and seminal vesicle, but a greater potency and intrinsic activity in increasing the weight of levator ani muscle. Particularly, compound of Formula XI at a dose as low as 0.3 mg/day, was able to maintain the levator ani muscle weight of castrated animals in the same level as that of intact animals. Thus, compound of Formula XI is a potent nonsteroidal anabolic agent with less androgenic activity but more anabolic activity than testosterone propionate.

### Example 11

### Synthesis of (S) Enantiomer of Compound of Formula XII

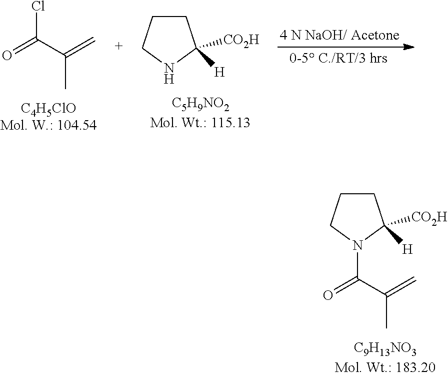

### Synthesis of (2R)-1-Methacryloylpyrrolidin-2-carboxylic Acid

A 72 L flask with a mechanical stirrer and inlet for inert atmosphere was set up in a cooling bath. The flask was placed under argon and charged with 5000 g (43.4 moles) of D-proline [ICN lot#7150E, ≥99%], 11.9 L of 4 N NaOH, and 12 L acetone. The mixture was cooled to 5° C. on an ice bath. A solution of 4548.8 g (43.5 moles) of methacryloyl chloride [Aldrich lot#12706HO, 98+%] in 12.0 L of acetone was prepared. The solution of methacryloyl chloride and 11.9 L of 4 N NaOH were added simultaneously to the reaction mixture in the 72 L flask. During the addition, the temperature was maintained less than 10° C. and the pH of the reaction mixture was maintained at greater than or equal to 10. The pH was maintained by adding the 4 N NaOH more slowly or more quickly depending on the pH of the solution. The addition time was approximately 2 h and 40 min. After the addition was complete, the reaction mixture was stirred overnight and allowed to warm to RT.

The acetone was removed on a rotary evaporator, and the aqueous mixture was extracted with t-butyl methyl ether (28.0 L). The mixture was then acidified with concentrated HCl (6568.1 g) to a pH of less than 2. The product was isolated by extraction into methylene chloride (3×20 L). The extracts were concentrated on a rotary evaporator. t-Butyl methyl ether (10 L) was added and concentrated on the rotary evaporator to perform a solvent exchange. Additional t-butyl methyl ether (10 L) was added to precipitate the product. Ice was charged to the rotary evaporator bath and the product was allowed to crystallize. The crystalline product was collected and isolated by filtration. The weight after drying in a vacuum oven at 50° C. was 4422.2 g (55.6% yield).

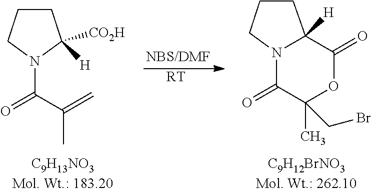

### Synthesis of (3R,8R)-3-Bromomethyl-3-methyl-tetrahydropyrolo[2,1-c][1,4]oxazine-1,4-dione

A 50 L flask was set up with a mechanical stirrer, inlet for inert atmosphere, and cooling capacity. The flask was placed under an argon atmosphere and was charged with 4410.0 g (24.1 moles) of (2R)-1-methacryloylpyrrolidin-2-carboxylic acid and 8.8 L of DMF. Then NBS (6409.6 g, 36.0 moles) was added slowly over a period of 2 h and 7 min. The reaction mixture was agitated for at least 8 h. Water (20.0 L) was added to precipitate the product. The product was allowed to stir for at least 4 h to crystallize. The crystalline product was collected and isolated by filtration. The weight after drying in a vacuum oven at 50° C. was 5532.1 g (87.7% yield).

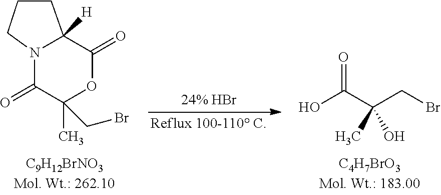

### Synthesis of (2R)-3-Bromo-2-hydroxy-2-methylpropanoic Acid

A 50 L flask was set up with a mechanical stirrer, inlet for inert atmosphere, and heating capacity. The flask was placed under an argon atmosphere and was charged with 5472.3 g (20.8 moles) of (3R,8R)-3-bromomethyl-3-methyl-tetrahydropyrolo[2,1-c][1,4]oxazine-1,4-dione and 14.175 L of deionized water and 14,118.4 g of 48% HBr. The reaction mixture was heated to 102° C. for 6 h, and allowed to cool to 31° C. Brine (20 L) was added to the reaction mixture and the product was extracted with 6×20.4 L of t-butyl methyl ether. The organic layers were combined and concentrated with the rotary evaporator. Toluene (4.0 L) was charged to the rotary evaporator. The product was dried by toluene distillation. The mixture was concentrated with the rotary evaporator. The product was recrystallized from toluene (45.0 L) by heating to 100° C. to dissolve the product. The flask was cooled on ice and the product was allowed to crystallize. The crystalline product was collected by filtration and washed with toluene (3.4 L). The weight after drying in a vacuum oven at 50° C. was 3107.0 g (81.3% yield).

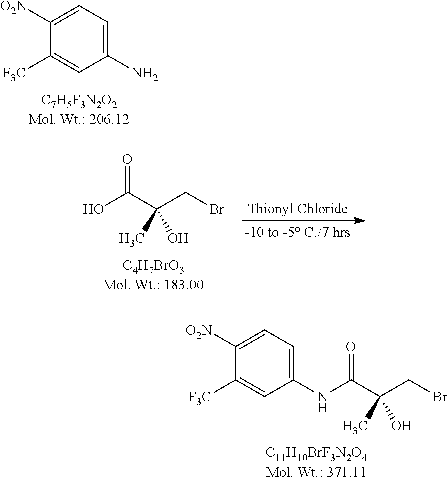

### Synthesis of N-[4-Nitro-3-(trifluoromethyl)phenyl]-(2R)-3-bromo-2-hydroxy-2-methylpropanamide

A 50 L flask was set up with a mechanical stirrer, inlet for inert atmosphere, and cooling capacity. The flask was placed under an argon atmosphere and was charged with 2961.5 g (16.2 moles) of (2R)-3-bromo-2-hydroxy-2-methylpropanoic acid and 9.0 L of THF. The flask was cooled on ice to less than 5° C. Thionyl chloride (1200 mL, 16.4 moles) dissolved in 6.0 L of THF was added slowly via an addition funnel to the reaction flask. The temperature of the reaction flask was maintained less than or equal to 10° C. The addition time was 1 h and 10 min. The reaction mixture was allowed to agitate for an additional 2 hand 50 min. Then a solution of 2359.4 g of (11.4 moles) of 4-nitro-3-trifluoromethylaniline (Aldrich, 98%) and 3.83 L of triethylamine in 6.0 L THF was added over a period of 3 h and 5 min. The temperature of the reaction flask was maintained less than or equal to 10° C. The ice bath was removed, and the reaction mixture was allowed to stir for 30 min. With a heating mantle, the reaction mixture was heated to 50° C. for 15 h and 10 min. After the reaction was complete as analyzed by TLC, the reaction mixture was cooled to less than 30° C. and 7.5 L of deionized water was added. The aqueous layer was removed and a second water wash (7.5 L) was performed. The organic layer was then washed three times with 10% bicarbonate (8.1 L) until the pH was greater than 7.

The solvent was removed on a rotary evaporator. Toluene (3.0 L) was added and then removed on the rotary evaporator to dry the crude product. The product was dissolved in 2.0 L of toluene at 65° C. Upon cooling the product crystallized. The crystalline product was collected and isolated by filtration. The wet cake was washed with 1.0 L of toluene. The weight after drying in a vacuum oven at 50° C. was 3751.0 g (70.3% yield).

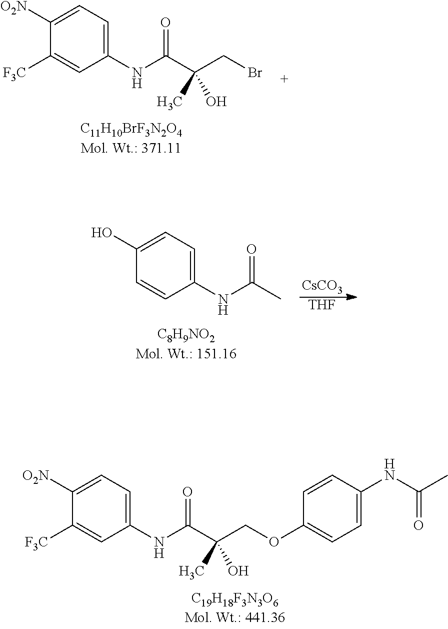

### Synthesis of S-3-(4-acetylaminophenoxy)-2-hydroxy-2-methyl-N-(4-nitro-3-trifluoromethylphenyl) propionamide (Compound of Formula XII)

A 22 L flask was set up with a mechanical stirrer, inlet for inert atmosphere, and cooling capacity. The flask was placed under an argon atmosphere and was charged with 1002.8 g (2.70 moles) of N-[4-nitro-3-(trifluoromethyl)phenyl]-(2R)-3-bromo-2-hydroxy-2-methylpropanamide, 4.0 L of THF, and 454.2 g (3.00 moles) of 4-acetamidophenol (Aldrich, 98%). While stirring, the flask was then charged with 1769.9 g of cesium carbonate (Aldrich, 99%). The flask was heated to reflux for at least 8 h, and the reaction monitored by TLC [silica gel, dichloromethane/hexane 3:1, Epoxide Rf=0.5]. When the reaction was complete, the flask was allowed to cool to RT.

Water was added to dissolve the carbonate and ethyl acetate was added to help with the phase separations. The aqueous phase was separated as waste. The organic phase was washed with a second portion of water. The organic layer was transferred to a rotary evaporator and the solvent was removed. The solvent was exchanged into ethanol by charging ethanol into the rotovap flask and removing some of the ethanol to remove all of the ethyl acetate. The ethanol solution was added to water to precipitate the product. The crude product was collected by filtration and washed with water. The product was transferred back to the rotary evaporator for crystallization. Ethyl acetate was charged to the rotovap flask to exchange the solvent into ethyl acetate. The ethyl acetate was removed under vacuum which dried the product. A minimum amount of ethyl acetate was added to dissolve the product at 60° C. t-Butyl methyl ether was added to crystallize the product. After cooling, the product was collected by filtration and washed with t-butyl methyl ether. The wet cake was added back to the rotary evaporator and ethanol was charged. A solvent exchange into ethanol removed the residual t-butyl methyl ether. Filtering the ethanol solution into water recrystallized the product. After stirring, the product was collected by filtration and washed with water. The weight after drying in a vacuum oven at 50° C. was 52%.

¹H NMR (300 MHz, DMSO-d₆) δ 10.62 (s, 1H, NH), 9.75 (s, 1H, NH), 8.56 (d, J=1.9 Hz, 1H, ArH), 8.36 (dd, J=9.1 Hz, J=1.9 Hz, 1H, ArH), 8.18 (d, J=9.1 Hz, 1H, ArH), 7.45-7.42 (m, 2H, ArH), 6.85-6.82 (m, 2H, ArH), 6.25 (s, 1H, OH), 4.17 (d, J=9.5 Hz, 1H, CHHₐ), 3.94 (d, J=9.5 Hz, 1H, CHH_{b}), 1.98 (s, 3H, Me), 1.43 (s, 3H, Me); ¹³C NMR (75 MHz, DMSO-d₆) δ 174.6 (C═O), 167.7, 154.2, 143.3, 141.6, 132.8, 127.4, 123.0, 122.7 (q, J=33.0 Hz), 122.1 (q, J=271.5 Hz), 120.1, 118.3 (q, J=6.0 Hz), 114.6, 74.9, 73.8, 23.8, 23.0.

### Example 12

### Preclinical Anabolic and Androgenic Pharmacology of Compound of Formula XII in Intact and Castrate Male Rats

Male Sprague-Dawley rats were purchased from Harlan Biosciences (Indianapolis, Ind.). The animals were maintained on a 12 h cycle of light and dark with food and water available ad libitum. All animal studies were reviewed and approved. Immature male Sprague-Dawley rats weighing 187 to 214 g were randomly distributed into 9 groups of 5 animals. One day before the initiation of drug treatment, groups 4 through 6 and groups 7 through 9 received unilateral or bilateral orchidectomy, respectively, via a midline scrotal incision. Groups 1 through 3 did not undergo surgery. All drugs given to animals were freshly prepared as solutions in polyethylene glycol 300 (PEG 300). Groups 4 and 7 received treatment with vehicle alone (i.e., PEG 300). Animals in groups 3, 6, and 9 received testosterone propionate (TP, 0.5 mg/day) via implantation of subdermal osmotic pumps (Model 2002, Durect Corporation, Palo Alto, Calif.). Animals in groups 2, 5, and 8 received compound of Formula XII (0.5 mg/day) via implantation of subdermal osmotic pumps. After 14 days of drug treatment, rats were weighed, anesthetized, and sacrificed. The ventral prostates, seminal vesicles, and levator ani muscle were removed and weighed. Osmotic pumps were also removed from animals to check for correct pump operation. The weights of all organs were normalized to body weight, and analyzed for any statistically significant differences between groups using single-factor ANOVA with the alpha value set a priori at p<0.05. The weights of prostates and seminal vesicles were used as indices for evaluation of androgenic activity, and the levator ani muscle weight was used to evaluate the anabolic activity. Statistical analyses of parameters from complete blood count or serum chemical profiling, wherever applicable, were performed by single-factor ANOVA with the alpha value set a priori at p<0.05.

### Results:

As shown in Table 10 in intact animals, compound of Formula XII decreased the size of the prostate to 79% and, of that observed in control animals, with no statistically significant changes in the size of the seminal vesicles or levator ani muscle. Compound of Formula XII decreased the size of the prostate and seminal vesicles to 75% and 79%, respectively, and increased the size of the levator ani muscle to 108% of that observed in untreated hemi-orchidectomized animals. These observations demonstrate that compound of Formula XII acts as a partial agonist in prostate and seminal vesicles and as a full agonist in levator ani muscle. No adverse pharmacologic effects were observed.

TABLE 10
Comparison of androgenic and anabolic effects of compound of
Formula XII and testosterone propionate (TP) on intact,
hemi-orchidectomized and castrated rats (% of intact control, n = 5).

| | | | Compound | |
|---|---|---|---|---|
| | | | of Formula XII | TP |
| Organs | | Control | (0.5 mg/day) | (0.5 mg/day) |
| | Intact | 100.00 ± 13.13 | 79.41 ± 9.32*^{†} | 97.45 ± 10.82 |
| Prostate | Hemi- | 86.42 ± 19.52 | 74.69 ± 8.44*^{†} | 98.57 ± 7.98 |
| | castrated | | | |
| | Castrated | 7.19 ± 1.25 | 32.55 ± 11.65*^{†‡} | 76.78 ± 10.43*^{‡} |
| Seminal | Intact | 100.00 ± 18.84 | 90.54 ± 12.10 | 103.95 ± 13.23 |
| Vesicle | Hemi- | 102.93 ± 7.47 | 78.55 ± 13.58^{†‡} | 114.19 ± 23.81 |
| | castrated | | | |
| | Castrated | 8.97 ± 1.23 | 16.47 ± 5.21*^{†‡} | 63.48 ± 17.05*^{‡} |
| | Intact | 100.00 ± 12.69 | 109.15 ± 14.68 | 95.61 ± 9.34 |
| Levator | Hemi- | 92.94 ± 7.83 | 108.10 ± 8.92^{‡} | 98.63 ± 10.47 |
| Ani | castrated | | | |
| | Castrated | 42.74 ± 5.22 | 100.65 ± 10.86^{‡} | 87.27 ± 10.25^{‡} |

*p < 0.05 compared to intact control group.
^{†}p < 0.05 compared to TP of same surgical status (i.e., intact, hemi-orchidectomized, or castrate).
^{‡}p < 0.05 compared to control group of same surgical status.

### Example 13

### Synthesis of (S) Enantiomer of Compound of Formula XIII

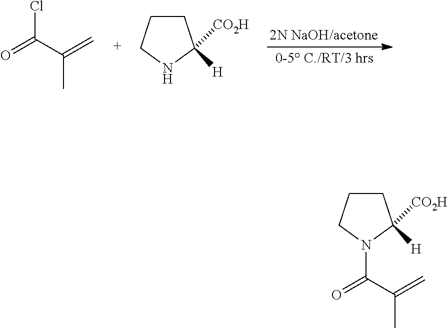

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, RT), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

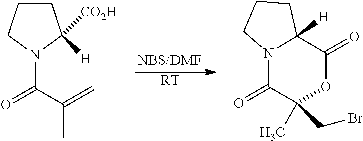

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

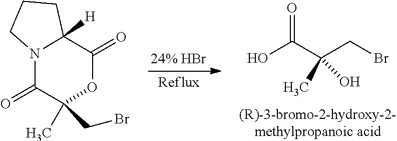

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.; ¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd. for C₄H₇BrO₃: C, 26.25, H, 3.86. Found: C, 26.28, H, 3.75.

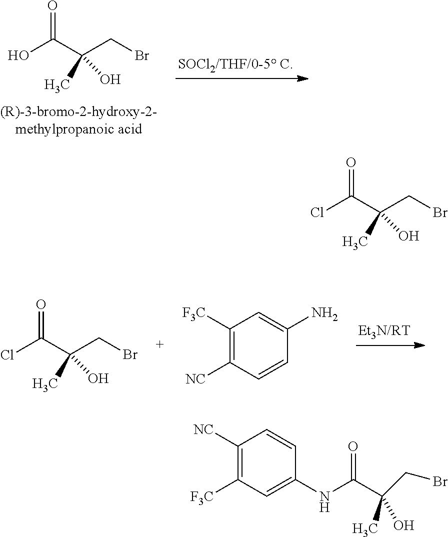

### Synthesis of (2R)-3-Bromo-N-[4-cyano-3-(trifluoromethyl)phenyl]-2-hydroxy-2-methylpropanamide

Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at RT. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M-H]⁻ 349.0. M.p.: 124-126° C.

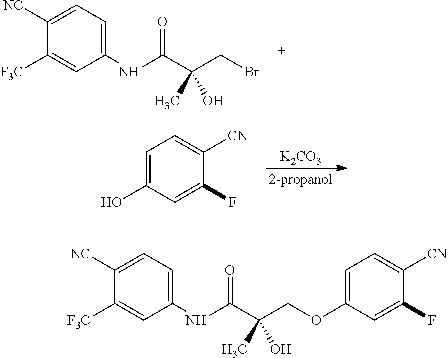

### Synthesis of (S)—N-(4-cyano-3-(trifluoromethyl)phenyl)-3-(4-cyano-3-fluorophenoxy)-2-hydroxy-2-methylpropanamide (Compound of Formula XIII)

A mixture of bromoamide (2R)-3-bromo-N-[4-cyano-3-(trifluoromethyl)phenyl]-2-hydroxy-2-methylpropanamide (2.0 g, 5.70 mmol) and anhydrous K₂CO₃ (2.4 g, 17.1 mmol) in 50 mL of acetone was heated to reflux for 2 h and then concentrated under reduced pressure to give a solid. The resulting solid was treated with 2-fluoro-4-hydroxybenzonitrile (1.2 g, 8.5 mmol) and anhydrous K₂CO₃ (1.6 g, 11.4 mmol) in 50 mL of 2-propanol and was heated to reflux for 3 h, then concentrated under reduced pressure to give a solid. The residue was treated with 100 mL of H20 and then extracted with EtOAc (2×100 mL). The combined EtOAc extracts were washed with 10% NaOH (4×100 mL) and brine, successively. The organic layer was dried over MgSO₄ and then concentrated under reduced pressure to give an oil which was crystallized from CH₂Cl₂/hexane to give 0.5 g (23%) of (S)—N-(4-cyano-3-(trifluoromethyl)phenyl)-3-(4-cyano-3-fluorophenoxy)-2-hydroxy-2-methylpropanamide as a colorless solid.

¹H NMR (CDCl₃/TMS) δ 1.63 (s, 3H, CH₃), 3.34 (bs, 1H, OH), 4.08 (d, J=9.17 Hz, 1H, CH), 4.50 (d, J=9.17 Hz, 1H, CH), 6.74-6.82 (m, 2H, ArH), 7.50-7.55 (m, 1H, ArH), 7.81 (d, J=8.50 Hz, 1H, ArH), 7.97 (q, J=2.03, 8.50 Hz, 1H, ArH), 8.11 (d, J=2.03 Hz, 1H, ArH), 9.12 (s, 1H, NH). Calculated Mass: 407.1, [M+Na]+430.0. Mp: 124-125° C.

### Example 14

### Preclinical Anabolic and Androgenic Pharmacology of Compound of Formula XIII in Intact and Castrate Male Rats

Anabolic and androgenic efficacy of compound of Formula XIII administered by daily oral gavage were tested. The S-isomer of the compound (compound of Formula XIII) was synthesized and tested as described herein.

### Materials and Methods:

Male Sprague-Dawley rats weighing approximately 200 g were purchased from Harlan Bioproducts for Science (Indianapolis, Ind.). The animals were maintained on a 12 h light/dark cycle with food (7012C LM-485 Mouse/Rat Sterilizable Diet, Harlan Teklad, Madison, Wis.) and water available ad libitum.

The test article for this study was weighed and dissolved in 10% DMSO (Fisher) diluted with PEG 300 (Acros Organics, NJ) for preparation of the appropriate dosage concentrations. The animals were housed in groups of 2 to 3 animals per cage. Animals were randomly assigned to one of seven groups consisting of 4 to 5 animals per group. Control groups (intact and ORX) were administered vehicle daily. Compounds of Formula XIII was administered via oral gavage at doses of 0.01, 0.03, 0.1, 0.3, 0.75, and 1 mg/day to both intact and ORX groups. Where appropriate, animals were castrated on day one of the study. Treatment with compound of Formula XIII began nine days post ORX and was administered daily via oral gavage for fourteen days.

The animals were sacrificed under anesthesia (ketamine/xyalzine, 87:13 mg/kg) and body weights were recorded. In addition, ventral prostate, seminal vesicles, and levator ani muscle were removed, individually weighed, normalized to body weight, and expressed as a percentage of intact control. Student's T-test was used to compare individual dose groups to the intact control group. Significance was defined a priori as a P-value <0.05. Ventral prostate and seminal vesicle weights were evaluated as a measure of androgenic activity, whereas levator ani muscle weight was evaluated as a measure of anabolic activity. Blood was collected from the abdominal aorta, centrifuged, and sera were frozen at −80° C. prior to determination of serum hormone levels. Serum luteinizing hormone (LH) and follicle stimulating hormone (FSH) concentrations were determined.

### Results:

A series of dose-response studies in intact and castrated rats in order to evaluate the potency and efficacy of compound of Formula XIII in both androgenic (prostate and seminal vesicles) and anabolic (levator ani muscle) tissue was conducted. In intact animals, compound of Formula XIII treatment resulted in decreases in the weight of both prostate and seminal vesicles while the levator ani muscle weight was significantly increased. Levator ani muscle weight following compound of Formula XIII treatment were 116%±7%, 134%±8%, 134%±21%, 134%±11%, 142%±10%, and 147%±10% of intact controls, following treatment with 0.01, 0.03, 0.1, 0.3, 0.75, and 1.0 mg/day dose groups, respectively. The prostate weights were 98%±21%, 99%±8%, 85%±18%, 98%±22%, 126%±17%, and 126%±17% of intact controls, following treatment with 0.01, 0.03, 0.1, 0.3, 0.75, and 1 mg/day, respectively. Similarly seminal vesicle weight was 115%±12%, 109%±17%, 106%±13%, 121%±11%, 157%±5%, and 136%±3% of intact controls following treatment with 0.01, 0.03, 0.1, 0.3, 0.75, and 1 mg/day, respectively. These results are significant since current androgen therapies are contraindicated in some patient populations due to the proliferative androgenic effects in prostate and breast tissues. However, many patients in these populations could benefit from the anabolic actions of androgens in muscle and bone. Since compound of Formula XIII exhibited tissue selective anabolic effects, it may be possible to treat patient groups in which androgens were contraindicated in the past.

In castrated (ORX) animals, prostate weights following compound of Formula XIII treatment were 24%±4%, 37%±9%, 50%±11%, 88%±16%, 132%±16%, and 118±12% of intact controls following doses of 0, 0.01, 0.03, 0.1, 0.3, 0.75, and 1 mg/day, respectively. Similarly, seminal vesicle weights were 15%±2%, 25%±9%, 67%±20%, 113%±6%, 155%±16%, and 160%±7% of intact controls, following doses of 0, 0.01, 0.03, 0.1, 0.3, 0.75, and 1 mg/day, respectively. Significant increases were seen in levator ani muscle weights of in all dose groups, when compared to intact controls. The levator ani muscle weights were 71%±4%, 101%±15%, 125%±20%, 126%±14%, 151±9%, and 143±17% of intact controls corresponding to 0, 0.01, 0.03, 0.1, 0.3, 0.75, and 1.0 mg/day dose groups, respectively. One unexpected finding was that administration of only 0.03 mg/day was able to fully restore levator ani muscle weight.

Comparable administration of testosterone propionate (TP) and S-3-(4-acetylaminophenoxy)-2-hydroxy-2-methyl-N-(4-nitro-3-trifluoromethylphenyl) propionamide (compound of Formula XII), maximally stimulated the levator ani muscle weight to 104% and 101%, respectively, indicating the significantly enhanced efficacy and potency of compound of Formula XIII. Taken together, these data show that compound of Formula XIII restores lost muscle mass, which in some embodiments, finds valuable application in patients with sarcopenia or cachexia, or other wasting diseases or disorders. Additionally, the antiproliferative effects of compound of Formula XIII on the prostate may allow some patient populations, in which androgens are currently contraindicated, access to anabolic agents. Eₘₐₓ values were obtained and were 147%±10%, 188%±135%, and 147%±10% for prostate, seminal vesicles, and levator ani, respectively. The ED₅₀ in prostate, seminal vesicles, and levator ani was 0.21±0.04, 0.2±0.04, and 0.03±0.01 mg/day, respectively.

### Example 15

### Synthesis of (S) Enantiomer of Compound of Formula XIV

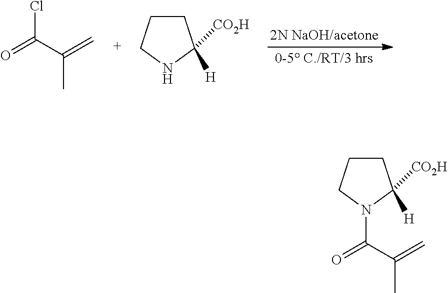

### (2R)-1-Methacryloylpyrrolidin-2-carboxylic Acid

D-Proline, 14.93 g, 0.13 mol) was dissolved in 71 mL of 2 N NaOH and cooled in an ice bath; the resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The pH of the mixture was kept at 10-11° C. during the addition of the methacryloyl chloride. After stirring (3 h, RT), the mixture was evaporated in vacuo at a temperature at 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102-103° C.; the NMR spectrum of this compound demonstrated the existence of two rotamers of the title compound. ¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8° (c=1, MeOH); Anal. Calcd. for C₉H₁₃NO₃: C, 59.00, H, 7.15, N, 7.65. Found: C, 59.13, H, 7.19, N, 7.61.

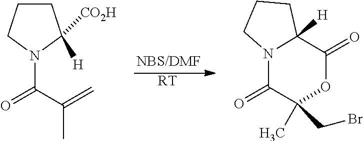

### (3R,8aR)-3-Bromomethyl-3-methyl-tetrahydro-pyrrolo[2,1-c][1,4]oxazine-1,4-dione

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried ˜34%) of the title compound as a yellow solid: mp 152-154° C.; ¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

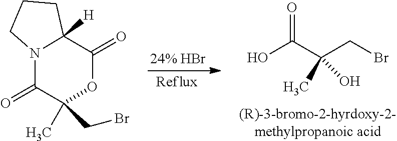

### (2R)-3-Bromo-2-hydroxy-2-methylpropanoic Acid

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃ (100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 107-109° C.;

¹H NMR (300 MHz, DMSO-d₆) δ 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm⁻¹; [α]_{D}²⁶+10.5° (c=2.6, MeOH); Anal. Calcd. for C₄H₇BrO₃: C, 26.25, H, 3.86. Found: C, 26.28, H, 3.75.

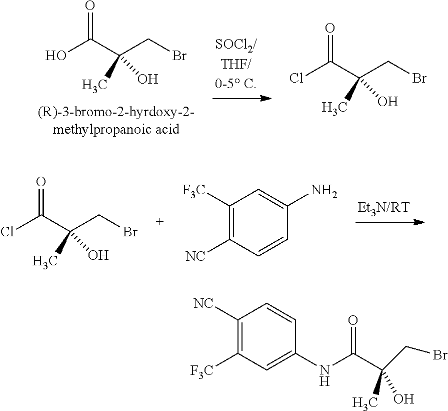

### Synthesis of (2R)-3-Bromo-N-[4-cyano-3-(trifluoromethyl)phenyl]-2-hydroxy-2-methylpropanamide

Thionyl chloride (46.02 g, 0.39 mol) was added dropwise to a cooled solution (less than 4° C.) of (R)-3-bromo-2-hydroxy-2-methylpropanoic acid (51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (40.0 g, 0.21 mol), 400 mL of THF were added and then the mixture was allowed to stir overnight at RT. The solvent was removed under reduced pressure to give a solid which was treated with 300 mL of H₂O, extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid, which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). Calculated Mass: 349.99, [M-H]⁻ 349.0. M.p.: 124-126° C.

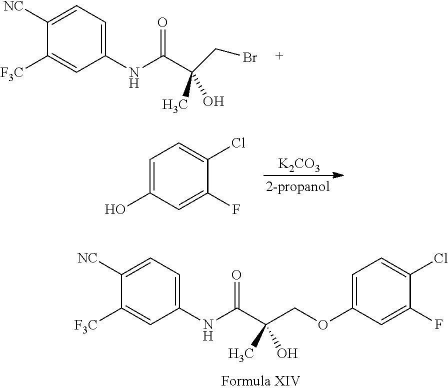

### Synthesis of (S)-3-(4-chloro-3-fluorophenoxy)-N-(4-cyano-3-(trifluoromethyl)phenyl)-2-hydroxy-2-methylpropanamide (Compound of Formula XIV)

A mixture of bromoamide (2R)-3-bromo-N-[4-cyano-3-(trifluoromethyl)phenyl]-2-hydroxy-2-methylpropanamide (2.0 g, 5.70 mmol) and anhydrous K₂CO₃ (2.4 g, 17.1 mmol) was heated to reflux for 2 h and then concentrated under reduced pressure to give a solid. The resulting solid was treated with 4-chloro-3-fluorophenol (1.3 g, 8.5 mmol) and anhydrous K₂CO₃ (1.6 g, 11.4 mmol) in 50 mL of 2-propanol and was heated to reflux for 3 h, then concentrated under reduced pressure to give a solid. The residue was treated with 100 mL of H₂O and then extracted with EtOAc (2×100 mL). The combined EtOAc extracts were washed with 10% NaOH (4×100 mL) and brine, successively. The organic layer was dried over MgSO₄ and then concentrated under reduced pressure to give an oil which was purified by column chromatography using EtOAc/hexane (50:50) to give a solid which was recrystallized from CH₂Cl₂/hexane to give 1.7 g (70.5%) of (S)-3-(4-chloro-3-fluorophenoxy)-N-(4-cyano-3-(trifluoromethyl)phenyl)-2-hydroxy-2-methylpropanamide as a colorless solid.

¹H NMR (CDCl₃/TMS) δ 1.60 (s, 3H, CH₃), 3.28 (s, 1H, OH), 3.98 (d, J=9.05 Hz, 1H, CH), 6.64-6.76 (m, 2H, ArH), 7.30 (d, J=8.67 Hz, 1H, ArH), 7.81 (d, J=8.52 Hz, 1H, ArH), 7.96 (q, J=2.07, 8.52 Hz, 1H, ArH), 8.10 (d, J=2.07 Hz, 1H, ArH), 9.10 (s, 1H, NH). Calculated Mass: [M-H]⁻ 414.9. Mp: 132-134° C.

### Example 16

### Preclinical Anabolic and Androgenic Pharmacology of Compound of Formula XIV in Intact and Castrate Male Rats

Anabolic and androgenic efficacy of compound of Formula XIV administered by daily oral gavage were tested. The S-isomer of compound of Formula XIV was synthesized and tested as described herein

### Materials and Methods:

Male Sprague-Dawley rats weighing approximately 200 g were purchased from Harlan Bioproducts for Science (Indianapolis, Ind.). The animals were maintained on a 12 h light/dark cycle with food (7012C LM-485 Mouse/Rat Sterilizable Diet, Harlan Teklad, Madison, Wis.) and water available ad libitum. The anabolic and androgenic activity of the compound of Formula XIV was studied in intact animals, acutely orchidectomized (ORX) animals and chronically (9 days) ORX rats.

The test article for this study was weighed and dissolved in 10% DMSO (Fisher) diluted with PEG 300 (Acros Organics, NJ) for preparation of the appropriate dosage concentrations. The animals were housed in groups of 2 to 3 animals per cage. Animals were randomly assigned to one of seven groups consisting of 4 to 5 animals per group. Control groups (intact and ORX) were administered vehicle daily. Compound of Formula XIV was administered via oral gavage at doses of 0.01, 0.03, 0.1, 0.3, 0.75, and 1 mg/day to both intact and ORX groups. Where appropriate, animals were castrated on day one of the study. Treatment with compound of Formula XIV began nine days post ORX and was administered daily via oral gavage for fourteen days.

The animals were sacrificed under anesthesia (ketamine/xyalzine, 87:13 mg/kg) and body weights were recorded. In addition, ventral prostate, seminal vesicles, and levator ani muscle were removed, individually weighed, normalized to body weight, and expressed as a percentage of intact control. Student's T-test was used to compare individual dose groups to the intact control group. Significance was defined a priori as a P-value <0.05. Ventral prostate and seminal vesicle weights were evaluated as a measure of androgenic activity, whereas levator ani muscle weight was evaluated as a measure of anabolic activity. Blood was collected from the abdominal aorta, centrifuged, and sera were frozen at −80° C. prior to determination of serum hormone levels. Serum luteinizing hormone (LH) and follicle stimulating hormone (FSH) concentrations were determined.

### Results:

A series of dose-response studies in intact and castrated rats in order to evaluate the potency and efficacy of compound of Formula XIV in both androgenic (prostate and seminal vesicles) and anabolic (levator ani muscle) tissue was conducted. In intact animals, compound of Formula XIV treatment resulted in decreases in the weight of both prostate and seminal vesicles while the levator ani muscle weight was significantly increased. Levator ani muscle weight following compound of Formula XIV treatment were 100%±10%, 98%±7%, 110%±5%, 110%±5%, 125%±10%, and 129%±10% of intact controls following doses of 0.01, 0.03, 0.1, 0.3, 0.75, and 1 mg/day, respectively. The prostate weights were 117%±20%, 98%±15%, 82%±20%, 62%±5%, 107%±30%, and 110%±14% of intact controls following doses of 0.01, 0.03, 0.1, 0.3, 0.75, and 1 mg/day, respectively. These results are significant since current androgen therapies are contraindicated in some patient populations due to the proliferative androgenic effects in prostate and breast tissues. However, many patients in these populations could benefit from the anabolic actions of androgens in muscle and bone. Since compound of Formula XIV exhibited tissue selective anabolic effects, it may be possible to treat patient groups in which androgens were contraindicated in the past.

In castrated, ORX animals, prostate weights following compound of Formula XIV treatment were 10%±3%, 12%±3%, 26%±7%, 39%±6%, 60%±14%, 88%±16%, and 123%±22% of intact controls following doses of 0, 0.01, 0.03, 0.1, 0.3, 0.75, and 1 mg/day, respectively. Similarly, seminal vesicle weights were 11%±1%, 11%±1%, 11%±1%, 27%±14%, 58%±18%, 86%±12%, and 100%±8% of intact controls following doses of 0, 0.01, 0.03, 0.1, 0.3, 0.75, and 1 mg/day, respectively. Significant increases were seen in levator ani muscle weights in all dose groups, when compared to intact controls. The levator ani muscle weights were 48%±8%, 50%±5%, 62%±6%, 89%±10%, 118%±6%, 134%±8% and 129%±14% of intact controls corresponding to 0, 0.01, 0.03, 0.1, 0.3, 0.75, and 1.0 mg/day dose groups, respectively.

Compound of Formula XIV exhibited anabolic muscle/prostate ratio in castrated rats of 4.10, 2.39, 2.28, 1.97, 1.53, 1.05 following doses of 0.01, 0.03, 0.1, 0.3, 0.75 and 1 mg/day, respectively.

Pharmacology results following 1 mg/day of compound of Formula XIV exhibited that prostate weight was 110%±14% of intact control and levator ani muscle weight was 129%±10% of intact control. Compound of Formula XIV maintained prostate weight following orchidectomy at 123±22% of intact controls and levator ani muscle weight at 129±14% of intact controls. A range of between 0.1 mg/day to 0.3 mg/day of compound of Formula XIV restored 100% of levator ani muscle weight, while between 39 to 60% prostate weight was restored.

### Example 17

### Preclinical Anabolic and Androgenic Pharmacology of Compounds of the Invention

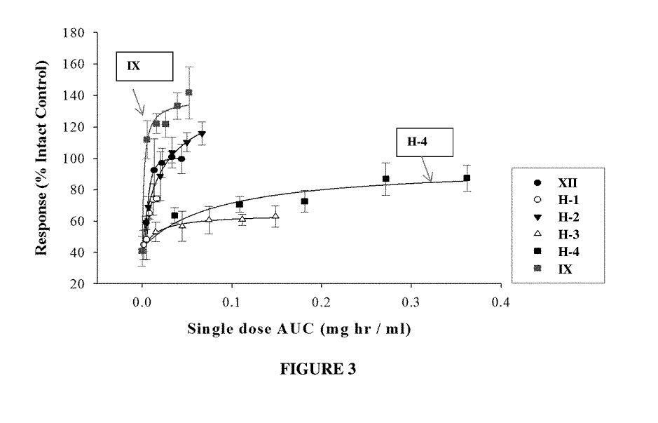

Hershberger assays as described above for Formulas VIII-XIV were also performed on compounds H-1, H-2, H-3, & H-4, as reported in FIG. 3, along with AR binding data in some cases in Table 11. The reported Eₘₐₓ values were calculated in Win Non-Lin®. Eₘₐₓ vs. AUC plots demonstrate that a spectrum of levator ani anabolic efficacies are possible with SARM compounds.

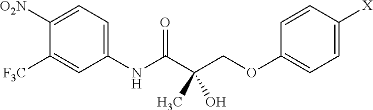

TABLE 11
Hershberger assays

| | | | Eₘₐₓ in levator |
|---|---|---|---|
| | | | ani muscle (% |
| Compound | | Kᵢ | of intact |
| ID | X | (nM) | control) |
| H-1 | F | 6.1 ± 0.1 | 75 ± 4 |
| H-2 | Cl | 8.6 ± 1.2 | 136 ± 9 |
| H-3 | Br | 13 ± 2 | 64 ± 4 |
| H-4 | I | 23 ± 2 | 95 ± 7 |

### Example 18

### Compound IX's Effect on Urge Incontinence

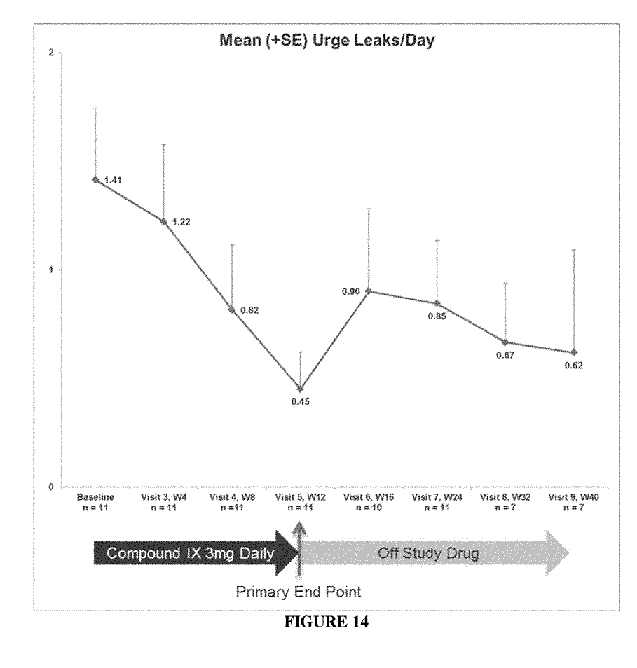

The clinical trial of Example 3 also demonstrated for the first time that Compound IX has effects not just on stress urinary incontinence, but also urge urinary incontinence (UUI). It was not expected to see any effect in UUI with Compound IX. The data showed that of the 18 patients in the initial trial, 11 patients reported >0 urge leaks/day at baseline. Unexpectedly, Compound IX reduced the median and mean number of urge leaks by ˜60-70% at the week 12 endpoint (Table 12). FIG. 14 provides mean urge leaks/day data over the course of the study in these 11 patients. At baseline the mean was 1.41 urge leaks per day which was reduced to 0.45 urge leaks per day at weak 12, representing a 68% decrease from baseline. Further, the effect on urge incontinence is durable for at least 7 months (N=7) with 0.62 urge leaks per day at 40 weeks (7 months after the last dose of Compound IX). It was unexpected to see any effect of Compound IX on urge incontinence much less such a dramatic and durable effect.

FIGS. 13A and 13B provide medical, epidemiologic and social aspects of aging (MESA) score for Compound IX. FIG. 13A (MESA stress) and FIG. 13B (MESA urge) show effects in stress and urge incontinence.

To summarize: while all of the women in the trial had predominant SUI, some also experienced urge urinary incontinence (UUI). 11 of the 18 women completing 12 weeks of treatment were determined to have both SUI and UUI at baseline, and these 11 women with mixed incontinence demonstrated a mean reduction in their UUI episodes of approximately 68 percent.

Urge leaks decreased from a mean of 1.41 leaks/day at baseline, to 0.45 leaks/day (FIG. 14);

9 of 11 women demonstrated a reduction in their number of UUI leaks, compared to baseline, with 8 of 11 demonstrating a clinically meaningful reduction in their UUI episodes per day of at least 50 percent (Table 12).

Median value of Medical, Epidemiologic and Social Aspects of Aging (MESA) scores for UUI (MESA urge) decreased from 56 percent to 22 percent (FIG. 13A).

TABLE 12
Number of Urge Incontinence Leaks per Day

| | A | B | C | D | E | F |
|---|---|---|---|---|---|---|
| 1 | Patients with >0 baseline leaks and a week 12 assesment: | | | | | |
| 2 | Pt | Baseline | | Week 12 | | |
| 3 | 2 | 2.33 | | 1.67 | | |
| 4 | 4 | 0.33 | | 0.67 | | |
| 5 | 5 | 0.33 | | 0 | | |
| 6 | 6 | 0.6 | | 0.33 | | |
| 7 | 7 | 3.67 | | 1 | | |
| 8 | 8 | 1.33 | | 0 | | |
| 9 | 10 | 0.33 | | 1 | | |
| 10 | 11 | 1 | | 0 | | |
| 11 | 13 | 1.66 | | 0 | | |
| 12 | 15 | 1.3 | | 0.3 | | |
| 13 | 18 | 2.67 | | 0 | | |
| 14 | | 1.3 | | 0.3 | Median | |
| 15 | | 1.41 | | 0.45 | Mean | |
| 16 | | | | | | |
| 17 | Other patients: | | | | | |
| 18 | Pt | Baseline | | Week 12 | | |
| 19 | 1 | 0 | | 0 | | |
| 20 | 3 | 0 | | 0 | | |
| 21 | 9 | 0 | | 0 | | |
| 22 | 12 | 5 | | | | |
| 23 | 14 | 0 | | 2.7 | | |
| 24 | 15 | 0 | | 0 | | |
| 25 | 17 | 0 | | 0 | | |
| 26 | | | | | | |

### Example 19

### Levator Ani Thickness and Urethral Sphincter Diameter by Quantitative MRI

In a continuation of the same study from Example 3, quantitative MRI was used to visualize the pelvic floor of 17 post-menopausal subjects with SUI. MRI was used to visualize and measure the mean thickness of the levator ani muscle, inner and outer diameter of the urethra, and the endometrial thickness of patients on trial at baseline and at the MRI visits approximately 12 weeks after baseline (exact times between baseline and second MRI are given). All data analysis used a significance value of 0.05 with one-tailed paired t-test. The mean change in each measurement and the statistical significance calculation for each value are given below:

### Mean Levator Ani Muscle Thickness:

### Difference Scores Calculations

### Mean: 1.15 mm

μ=0

S²=SS/df=44.38/(17−1)=2.77

S²_{M}=S²/N=2.77/17=0.16

S_{M}=√S²_{M}=√0.16=0.40

### T-Value Calculation

t=(M−μ)/S_{M}=(1.15−0)/0.40=2.84

The value of t is 2.839651. The value of p is 0.005916. The result is significant at p≤0.05.

This means the levator ani muscle thickness was significantly thicker (i.e. stronger) after the use of Compound IX than before, which would be favorable in the post-menopausal SUI patient population and may help to rationalize the 100% response rates in SUI (N=18) and UUI (for those reporting urge leaks at baseline (N=11)), and the durability of both effects in the first nine and seven patients, respectively, reaching 40 weeks.

### Urethra Inner Diameter:

### Difference Scores Calculations

### Mean: 0.07 cm

μ=0

S²=SS/df=0.12/(17−1)=0.01

S²_{M}=S²/N=0.01/17=0.00

S_{M}=√S²_{M}=√0.00=0.02

### T-Value Calculation

t=(M−μ)/S_{M}=(0.07−0)/0.02=3.43

The value of t is 3.428571. The value of p is 0.001723. The result is significant at p≤0.05.

The inner diameter of the urethra includes the smooth muscle, submucosa, and mucosa. This was significantly thicker after the use of Compound IX than before, which is favorable and suggests increased tone/strength of the underlying muscle complex.

### Urethra Outer Diameter:

### Difference Scores Calculations

### Mean: 0.07 cm

μ=0

S²=SS/df=0.08/(17−1)=0.00

S²_{M}=S²/N=0.00/17=0.00

S_{M}=√S²_{M}=√0.00=0.02

### T-Value Calculation

t=(M-μ)/S_{M}=(0.07−0)/0.02=4.24

The value of t is 4.242641. The value of p is 0.00031. The result is significant at p≤0.05.

The outer diameter includes the striated urethral sphincter, smooth muscle, submucosa, and mucosa. This was significantly thicker after the use of Compound IX than before, which is favorable and suggests increased tone/strength of the underlying muscle complex. The urethral data may also help to rationalize the 100% response rates in SUI (N=18) and UUI (for those reporting urge leaks at baseline (N=11)), and the durability of both responses in the first nine and seven patients, respectively, reaching 40 weeks.

### Endometrial Thickness:

### Difference Scores Calculations

### Mean: 0.07 mm

μ=0

S²=SS/df=6.93/(14−1)=0.53

S²_{M}=S²/N=0.53/14=0.04

S_{M}=√S²_{M}=√0.04=0.20.

### T-Value Calculation

t=(M−μ)/S_{M}=(0.07−0)/0.20=0.37

The value of t is 0.366088. The value of p is 0.360094. The result is not significant as the p value is above the threshold of p≤0.05.

Endometrial thickness was not significantly changed before and after drug, which is favorable since it demonstrates a lack of potential hormonal effect on endometrium.

The results showed a significant increase in several important measurements and support the mechanism of action of Compound IX on the pelvic floor.

At week 12, mean levator ani muscle thickness increased 1.15 mm (p=0.006) from a baseline measurement of 4.79 mm;

At week 12, mean inner urethral muscle diameter increased 0.7 mm (p=0.002) from a baseline measurement of 10.7 mm; and

At week 12, mean outer urethral muscle diameter increased 0.7 mm (p=0.0003) from a baseline of 15.4 mm.

The results showed statistically significant increases and support the mechanism of action of Compound IX on the pelvic floor. Further, the effect on the inner and outer urethra indicates that Compound IX has effects not just on the striated muscle of the levator ani and/or striated muscle of the urethra, but also on the urethral smooth muscle. Moreover, UUI is often associated with a feeling of urgency when there is incomplete blockage of urine at the bladder neck causing posterior urethral filling at the junction between the bladder and the urethral canal. The ability of Compound IX to increase the bulk, and presumably strength, of the pelvic floor muscles including the urethra helps to rationalize how Compound IX can produce the unexpectedly efficacious and durable effects on SUI and UUI. Further, muscle hypertrophy as a mechanism of action in SUI and UUI is not rapidly reversible like the pharmacological approaches to other urinary incontinences. E.g., beta3-agonists or muscarinic antagonists do not affect muscle structure but rather cause muscle relaxation through their effects on the innervation of the bladder muscles. As these pharmacological agents are cleared from the body, the muscles of the bladder return to their untreated states and UI is expected to return. In contrast, SARMs do not act through adrenergic or muscarinic innervation of the bladder but rather cause muscle hypertrophy in the pelvic floor muscles supporting the bladder. Pelvic floor atrophy which took decades to develop before treatment is not rapidly reversible upon withdrawal of the SARM, possibly allowing for the unprecedented efficacies and durations with a urinary incontinence drug in this proof-of-concept SUI trial with post-hoc UUI analysis.

Table 13 provides the individual measurements from the quantitative MRI data for these seventeen patients. The data demonstrated that Compound IX increased levator ani width and urethral inner and outer diameter in these post-menopausal humans, suggesting that Compound IX affected the architecture of the pelvic floor. In view of the unexpectedly robust clinical responses with regard to Compound IX treated subjects who experienced decreased SUI and UUI and mixed incontinence episodes (the latter two of which were not expected to necessarily respond to Compound IX) and the unexpected durability of these clinical responses with Compound IX; and further in view of the changed architecture of the pelvic floor to help rationalize the clinical responses, Compound IX is an unexpectedly promising agent for post-menopausal SUI or UUI, or mixed incontinence. Further, the effects on SUI would have to be considered unexpected since currently there are no approved pharmacotherapeutics for SUI despite the large affected population and the multiple non-pharmacotherapeutic treatments.

TABLE 13

| | Mean levator | | | | | Mean levator | | | |
|---|---|---|---|---|---|---|---|---|---|
| | ani (LA) | | | | | ani (LA) | | | |
| | thickness at | Urethra | Urethra | | | thickness at | | Urethra | Urethra |
| | the level of | inner | outer | | Follow up MRI | the level of | | inner | outer |
| | the proximal | diameter | diameter | Endometrial | date (days after | the proximal | Endometrial | diameter | diameter |
| | urethra (mm) | (cm) | (cm) | stripe (mm) | trial day 0) | urethra (mm) | stripe (mm) | (cm) | (cm) |
| 1 | 4 | 0.9 | 1.5 | 4 | 89 | 4 | 3 | 1 | 1.6 |
| 2 | 3 | 1.3 | 1.6 | 2 | 99 | 4 | 2 | 1.4 | 1.7 |
| 3 | 5 | 1 | 1.6 | 2 | 87 | 5 | 2 | 1.1 | 1.6 |
| 4 | 5 | 1.2 | 1.8 | 2 | 88 | 5 | 2 | 1.2 | 1.8 |
| 5 | 6 | 1 | 1.4 | 2 | 95 | 6 | 2 | 1 | 1.6 |
| 6 | 5 | 0.8 | 1.3 | 2 | 92 | 4 | 2 | 1.1 | 1.4 |
| 7 | 1 | 0.9 | 1.2 | | 97 | 1 | | 0.9 | 1.2 |
| 8 | 4.5 | 1.3 | 1.7 | 2 | 90 | 6.5 | 2 | 1.3 | 1.7 |
| 9 | 5.5 | 1.2 | 1.9 | 2 | 89 | 6 | 2 | 1.3 | 2 |
| 10 | 3 | 1.2 | 1.5 | 4 | 89 | 5 | 3 | 1.3 | 1.6 |
| 11 | 5 | 1.3 | 1.9 | | 94 | 8 | | 1.3 | 1.9 |
| 12 | 8 | 1.2 | 1.5 | | 97 | 10.5 | | 1.2 | 1.6 |
| 13 | 6 | 0.9 | 1.2 | 5 | 113 | 7 | 5 | 1 | 1.3 |
| 14 | 6.5 | 1.2 | 1.6 | 2 | 106 | 7 | 4 | 1.4 | 1.8 |
| 15 | 4 | 1.1 | 1.3 | 2 | 84 | 6 | 2 | 1.1 | 1.3 |
| 16 | 4 | 0.9 | 1.8 | 2 | 73 | 10 | 2 | 0.9 | 1.8 |
| 18 | 6 | 0.9 | 1.3 | 3 | 98 | 6 | 4 | 1 | 1.4 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating, preventing, suppressing or inhibiting stress urinary incontinence, urge urinary incontinence or a combination thereof in a postmenopausal female subject, comprising administering to said subject a composition comprising a SARM compound of Formula IX: 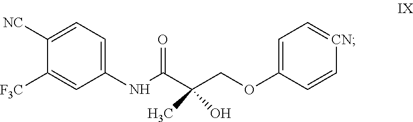
or its isomer, hydrate, pharmaceutically acceptable salt, pharmaceutical composition or any combination thereof.

2. The method according to claim 1, wherein said administering comprises a daily dose of 1 to 3 mg.

3. The method according to claim 1, wherein the administering step is up to 12 weeks.

4. The method according to claim 2, wherein the administering step is up to 12 weeks.

5. The method according to claim 1, wherein the administering step is by intermittent dosing cycles.

6. The method according to claim 1, wherein the administering step consists of only one dose cycle which lasts up to 12 weeks.

7. The method according to claim 1, wherein the subject suffers from stress urinary incontinence and urge urinary incontinence.

8. The method according to claim 1, wherein the composition comprises a 1 mg daily dose of said compound.

9. The method according to claim 1, wherein the composition comprises a 3 mg daily dose of said compound.

10. The method according to claim 2, wherein the duration of response following completion of the treatment is at least seven months post-treatment.

11. The method according to claim 4, wherein the duration of response following completion of the treatment is at least seven months post-treatment.

12. A method of reducing the occurrence or lessening the severity of at least one of the following symptoms in a postmenopausal female subject suffering from stress urinary incontinence, urge urinary incontinence or a combination thereof: (i) average daily frequency of urination; (ii) average nightly frequency of urination; (iii) total urinary incontinence episodes; (iv) stress incontinence episodes; and (v) urinary urgency episodes; comprising administering a SARM compound of Formula IX: 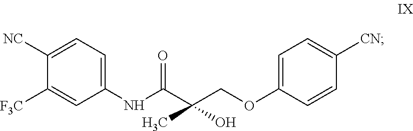 [SMILES: C[C@](O)(COc1ccc(C#N)cc1)C(=O)Nc1ccc(C#N)c(C(F)(F)F)c1]
or its isomer, hydrate, pharmaceutically acceptable salt, pharmaceutical composition or any combination thereof.

13. The method according to claim 12, wherein said administering comprises a daily dose of 1 to 3 mg.

14. The method according to claim 12, wherein the administering step is up to 12 weeks.

15. The method according to claim 13, wherein the administering step is up to 12 weeks.

16. The method according to claim 12, wherein the administering step is by intermittent dosing cycles.

17. The method according to claim 12, wherein the administering step consists of only one dose cycle which lasts up to 12 weeks.

18. The method according to claim 12, wherein the subject suffers from stress urinary incontinence and urge urinary incontinence.

19. The method according to claim 12, wherein the composition comprises a 1 mg daily dose of said compound.

20. The method according to claim 12, wherein the composition comprises a 3 mg daily dose of said compound.

21. The method according to claim 13, wherein the duration of response following completion of the treatment is at least seven months post-treatment.

22. The method according to claim 15, wherein the duration of response following completion of the treatment is at least seven months post-treatment.
